(19) United States
(12) Patent Application Publication
(10) Pub. No.: US 2022/0378707 A1
(43) Pub. Date: Dec. 1, 2022

(54) 1-(((2S,3S,4S)-3-ETHYL-4-FLUORO-5-OXOPYRROLIDIN-2-YL)METHOXY)-7-METHOXYISOQUINOLINE-6-CARBOXAMIDE COMBINATIONS AND ORAL DOSAGE FORMS

(71) Applicant: Pfizer Inc., New York, NY (US)

(72) Inventors: Allan Joseph Hutchins, Mystic, CT (US); Bruce Clinton MacDonald, Niantic, CT (US); Vikram Rama Rao, Maynard, MA (US); Degui Tang, Salem, CT (US); Weili Yu, Pawcatuck, CT (US)

(73) Assignee: Pfizer Inc., New York, NY (US)

(21) Appl. No.: 17/755,601

(22) PCT Filed: Nov. 11, 2020
(86) PCT No.: PCT/IB2020/060627

§ 371 Date: May 3, 2022

Related U.S. Application Data
(60) Provisional application No. 63/024,174, filed on May 13, 2020.
(60) Provisional application No. 62/935,450, filed on Nov. 14, 2019.

(51) Int. Cl. A61K 9/20; A61K 9/00; A61K 31/4725; A61K 9/28; A61K 31/519

(52) U.S. Cl. CPC A61K 9/2054; A61K 9/2018; A61K 9/2009; A61K 9/2027; A61K 9/2031; A61K 9/0004; A61K 31/4725; A61K 9/2866; A61K 9/2853; A61K 31/519; A61K 9/0053

(57) ABSTRACT

The present invention is related to the discovery of new oral dosage formulations and combination therapies for 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, for treating conditions ameliorated by inhibition of IRAK4.

### FIELD OF THE INVENTION

The present invention relates to the discovery of new oral dosage formulations for 1-(((2S,3S,4S)-3-ethyl-4-fluoro-5-oxopyrrolidin-2-yl)methoxy)-7-methoxyisoquinoline-6-carboxamide (PF-06650833) for treating or preventing immune, autoimmune, and inflammatory diseases in patients. The present invention also relates to combination therapies comprising 1-(((2S,3S,4S)-3-ethyl-4-fluoro-5-oxopyrrolidin-2-yl)methoxy)-7-methoxyisoquinoline-6-carboxamide for treating or preventing immune, autoimmune, and inflammatory diseases in patients.

### BACKGROUND OF THE INVENTION

1-(((2S,3S,4S)-3-Ethyl-4-fluoro-5-oxopyrrolidin-2-yl)methoxy)-7-methoxyisoquinoline-6-carboxamide is a selective, reversible inhibitor of interleukin (IL)-1 receptor associated kinase 4 (IRAK4) that is being developed for the treatment of conditions ameliorated by inhibition of IRAK4. IRAK4 is activated by the IL-1 family receptors (IL-1R, IL-18R, and IL-33R), as well as the Toll-like receptors. Inhibition of IRAK4 blocks the production of inflammatory cytokines such as type I interferons, tumor necrosis factor, IL-1, IL-6, and IL-12 that are key drivers of autoimmune and inflammatory diseases. Accordingly, IRAK4 inhibitors are attractive therapeutic targets for immune, autoimmune, and inflammation diseases.

The formulation of 1-(((2S,3S,4S)-3-Ethyl-4-fluoro-5-oxopyrrolidin-2-yl)methoxy)-7-methoxyisoquinoline-6-carboxamide used in the Phase II study for treating rheumatoid arthritis (RA) was a swellable core technology (SCT) modified release (MR) tablet (MR-FORM1). The SCT tablets were composed of two layers, with one layer containing PF-06650833 and the other layer containing excipients that release/push PF-06650833 out of the tablet that is coated. The unit dose strengths achievable using SCT were limited to 20 mgs and 100 mgs necessitating administration of multiple tablets to patients to achieve higher dosages. Therefore, a need existed for the discovery of a formulation with higher unit dosage strengths of PF-06650833 to reduce administration of multiple tablets to patients thereby providing the potential to improve patient compliance.

The present invention is directed to new extrudable core system (ECS) single-layer MR tablets where PF-06650833 and excipients, which release/push PF-06650833 out of the tablet, are blended together in an active core that is coated. The single-layer structure of the ECS (as oppose to the bi-layer of the SCT) enables the manufacture of tablets with a higher 200 mg unit dosage strength of PF-06650833 while maintaining comparable dissolution rates with the lower dosage SCT MR-FORM1 tablets. The ECS 200 mg tablets may reduce tablet burden and thereby enhance patient compliance for treatments that require higher doses of PF-06650833.

The present invention is also directed to pharmaceutical combinations comprising an IRAK4 inhibitor and a JAK inhibitor for treating immune, autoimmune, and inflammatory diseases.

### SUMMARY OF THE INVENTION

The present invention provides an oral dosage formulation comprising 200 mgs of 1-(((2S,3S,4S)-3-ethyl-4-fluoro-5-oxopyrrolidin-2-yl)methoxy)-7-methoxyisoquinoline-6-carboxamide or a pharmaceutically acceptable salt thereof.

In another embodiment, the present invention provides a method of treating or preventing an immune, autoimmune, or inflammatory disease in a patient comprising administering orally to the patient in need of such treatment one or more tablets wherein the tablets comprise 200 mgs of 1-(((2S,3S,4S)-3-ethyl-4-fluoro-5-oxopyrrolidin-2-yl)methoxy)-7-methoxyisoquinoline-6-carboxamide or a pharmaceutically acceptable salt thereof taken once daily, wherein more than one tablet is taken simultaneously or in sequence.

In another embodiment, the present invention provides a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof.

In another embodiment, the present invention provides a method of treating or preventing an immune, autoimmune, or inflammatory disease in a patient comprising administering orally to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, wherein the combination is administered simultaneously or sequentially once daily.

In another embodiment, the present invention provides a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, for simultaneous or sequential administration once daily, for use in treating or preventing an immune, autoimmune, or inflammatory disease.

In another embodiment, the present invention provides a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 3-((3R,4R)-4-methyl-3-(methyl(7H-pyrrolo[2,3-d]pyrimidin-4-yl)amino)piperidin-1-yl)-3-oxopropanenitrile (tofacitinib), or a pharmaceutically acceptable salt thereof.

In another embodiment, the present invention provides a method of treating or preventing an immune, autoimmune, or inflammatory disease in a patient comprising administering orally to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or pharmaceutically acceptable salt thereof, wherein the combination is administered simultaneously or sequentially once daily.

In another embodiment, the present invention provides a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, for simultaneous or sequential oral administration once daily, for use in treating or preventing an immune, autoimmune, or inflammatory disease.

In another embodiment, the present invention provides one or more 100 mg MR-FORM3 tablets for oral administration once daily, wherein more than one tablet is taken simultaneously or in sequence, for use in treating or preventing an immune, autoimmune, or inflammatory disease.

In another embodiment, the present invention provides one or more 200 mg MR-FORM3 tablets for oral administration once daily, wherein more than one tablet is taken simultaneously or in sequence, for use in treating or preventing an immune, autoimmune, or inflammatory disease.

In another embodiment, the present invention provides a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, for simultaneous or sequential administration once daily, for use in treating or preventing an immune, autoimmune, or inflammatory disease.

In another embodiment, the present invention provides a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, for simultaneous or sequential oral administration once daily, for use in treating or preventing an immune, autoimmune, or inflammatory disease.

### BRIEF DESCRIPTION OF THE DRAWINGS

FIG. 1A provides the dissolution profile for the 200 mg MR-FORM2 tablets.

FIG. 1B provides the dissolution profile for the 100 mg MR-FORM1 tablets.

FIG. 2 provides the plasma concentration-time profile for 1-(((2S,3S,4S)-3-ethyl-4-fluoro-5-oxopyrrolidin-2-yl)methoxy)-7-methoxyisoquinoline-6-carboxamide (PF-06650833) with and without co-administration of 1-((2S,5R)-5-((7H-pyrrolo[2,3-d]pyrimidin-4-yl)amino)-2-methylpiperidin-1-yl)prop-2-en-1-one (PF-06651600).

FIG. 3 compares the median plasma concentration-time profiles following oral dosing in healthy humans of: 100 mg MR-FORM1 tablet (fasted) versus 100 mg MR-FORM2 tablet (fasted); four 100 mg MR-FORM1 tablets (fasted) versus two 200 mg MR-FORM2 tablets (fasted); and a 100 mg MR-FORM2 tablet (fed) versus two 200 mg MR-FORM2 tablets (fed).

FIG. 4 compares the median plasma concentration-time profiles following oral dosing in healthy humans of 100 mg MR-FORM1 tablet (fasted) versus 100 mg MR-FORM2 tablet (fasted).

FIG. 5 compares the median plasma concentration-time profiles following oral dosing in healthy humans of four 100 mg MR-FORM1 tablets (fasted) versus two 200 mg MR-FORM2 tablets (fasted).

FIG. 6 compares the median plasma concentration-time profiles following oral dosing in healthy humans of a 100 mg MR-FORM2 tablet (fasted) versus a 100 mg MR-FORM2 tablet (fed).

FIG. 7 compares the median plasma concentration-time profiles following oral dosing in healthy humans of two 200 mg MR-FORM2 tablets (fasted) versus two 200 mg MR-FORM2 tablets (fed).

FIGS. 8, 9, and 10 provide explanations of BioMAP terms, pictorials, and graphical representations.

FIG. 11 provides a description of the BioMAP systems used to evaluate the biological activity of 1-(((2S,3S,4S)-3-ethyl-4-fluoro-5-oxopyrrolidin-2-yl)methoxy)-7-methoxyisoquinoline-6-carboxamide, 1-((2S,5R)-5-((7H-pyrrolo[2,3-d]pyrimidin-4-yl)amino)-2-methylpiperidin-1-yl)prop-2-en-1-one, and tofacitinib.

FIG. 12 provides the BioMAP analysis of 1-(((2S,3S,4S)-3-ethyl-4-fluoro-5-oxopyrrolidin-2-yl)methoxy)-7-methoxyisoquinoline-6-carboxamide at concentrations of 500 nM, 170 nM, 56 nM, and 19 nM.

FIG. 13 provides the BioMAP analysis of 1-((2S,5R)-5-((7H-pyrrolo[2,3-d]pyrimidin-4-yl)amino)-2-methylpiperidin-1-yl)prop-2-en-1-one at concentrations of 5400 nM, 1600 nM, 600 nM, and 200 nM.

FIG. 14 provides the BioMAP analysis of tofacitinib at concentrations of 1000 nM, 330 nM, 110 nM, and 37 nM.

FIG. 15 provides the BioMAP analysis in the 3C, SAg, and HDF3CGF systems for 1-(((2S,3S,4S)-3-ethyl-4-fluoro-5-oxopyrrolidin-2-yl)methoxy)-7-methoxyisoquinoline-6-carboxamide at concentrations of 500 nm, 170 nM, 56 nM, and 10 nM.

FIG. 16 provides the BioMAP analysis in the 3C, SAg, and HDF3CGF systems for Tofacitinib at concentrations of 1000 nm, 330 nM, 110 nM, and 37 nM.

FIG. 17 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (500 nM); Tofacitinib (1000 nM); and the combination thereof.

FIG. 18 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (500 nM); Tofacitinib (330 nM); and the combination thereof.

FIG. 19 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (500 nM); Tofacitinib (110 nM); and the combination thereof.

FIG. 20 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (500 nM); Tofacitinib (37 nM); and the combination thereof.

FIG. 21 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (170 nM); Tofacitinib (1000 nM); and the combination thereof (167 nM+1000 nM).

FIG. 22 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (170 nM); Tofacitinib (330 nM); and the combination thereof (167 nM+330 nM).

FIG. 23 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (170 nM); Tofacitinib (110 nM); and the combination thereof (167 nM+110 nM).

FIG. 24 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (170 nM); Tofacitinib (37 nM); and the combination thereof (167 nM+37 nM).

FIG. 25 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (56 nM); Tofacitinib (1000 nM); and the combination thereof.

FIG. 26 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (56 nM); Tofacitinib (330 nM); and the combination thereof.

FIG. 27 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (56 nM); Tofacitinib (110 nM); and the combination thereof.

FIG. 28 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (56 nM); Tofacitinib (37 nM); and the combination thereof.

FIG. 29 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (19 nM); Tofacitinib (1000 nM); and the combination thereof.

FIG. 30 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (19 nM); Tofacitinib (330 nM); and the combination thereof.

FIG. 31 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (19 nM); Tofacitinib (110 nM); and the combination thereof.

FIG. 32 provides the BioMAP comparative analysis in the 3C, SAg, and HDF3CGF systems for: 1-(((2S,3S,4S)-3-Ethyl-4-fluoro-5-oxopyrrolidin-2-yl)methoxy)-7-methoxyisoquinoline-6-carboxamide (19 nM); Tofacitinib (37 nM); and the combination thereof.

FIGS. 33, 34, 35, 36, 37, 38, and 39 provide the Log₁₀ ratios in the 3C, SAg, and HDF3CGF systems for: 1-(((2S,3S,4S)-3-ethyl-4-fluoro-5-oxopyrrolidin-2-yl)methoxy)-7-methoxyisoquinoline-6-carboxamide at concentrations of 500 nM, 170 nM, 56 nM, and 19 nM; tofacitinib at concentrations of 1000 nM, 330 nM, 110 nM, and 37 nM; and combinations thereof.

FIG. 40 provides the Significance Envelope Log₁₀ ratios for the 3C, SAg, and HDF3CGF systems.

FIG. 41 demonstrates the instability of the 100 mg MR-FORM2 tablets at relative humidity greater than 45%.

FIG. 42 demonstrates the instability of tablets comprising an active layer identical to the 200 mg MR-FORM2 active layer with a coating applied to the active layer comprising 60% cellulose acetate and 40% hydroxylpropylcellulose resulting in unpredictable/erratic dissolution profiles.

### DETAILED DESCRIPTION OF THE INVENTION

In another embodiment, the present invention provides an oral dosage formulation tablet or capsule comprising 1-400 mgs of 1-(((2S,3S,4S)-3-ethyl-4-fluoro-5-oxopyrrolidin-2-yl)methoxy)-7-methoxyisoquinoline-6-carboxamide (PF-06650833), or an equivalent amount of PF-06650833 in the form of a pharmaceutically acceptable salt thereof, and at least one pharmaceutically acceptable excipient, diluent, or carrier.

In another embodiment, the present invention provides an oral dosage formulation tablet or capsule comprising 50-300 mgs of 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and at least one pharmaceutically acceptable excipient, diluent, or carrier.

In another embodiment, the present invention provides an oral dosage formulation tablet or capsule comprising 100-200 mgs of 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and at least one pharmaceutically acceptable excipient, diluent, or carrier.

In another embodiment, the present invention provides an oral dosage formulation tablet or capsule comprising 100 mgs of 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and at least one pharmaceutically acceptable excipient, diluent, or carrier.

In another embodiment, the present invention provides an oral dosage formulation tablet or capsule comprising 200 mgs of 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and at least one pharmaceutically acceptable excipient, diluent, or carrier.

In another embodiment, the present invention provides an oral dosage formulation tablet or capsule comprising 200 mgs of 1-(((2S,3S,4S)-3-ethyl-4-fluoro-5-oxopyrrolidin-2-yl)methoxy)-7-methoxyisoquinoline-6-carboxamide and at least one pharmaceutically acceptable excipient, diluent, or carrier.

In another embodiment, the present invention provides an oral dosage ECS single layer modified release tablet comprising an active core and a coating applied to the active core wherein the active core comprises 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants, and wherein the coating comprises an osmotic membrane and a plasticizer.

In another embodiment, the present invention provides an oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein PF-06650833 is unmilled.

In another embodiment, the present invention provides an oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein dissolution of the tablet is 80%±10% after eight hours, preferably 80%±5% after eight hours.

In another embodiment, the present invention provides an oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein dissolution of the tablet is 80%±10% after eight hours, preferably 80%±5%, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein the tablet has NMT 0.05% total degradant products at 25° C./60% RH after 13 months.

In another embodiment, the present invention provides an oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein the tablet has NMT 0.05% total degradant products at 40° C./75% RH after 6 months.

In another embodiment, the present invention provides an oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein PF-06650833 is unmilled, wherein dissolution of the tablet is 80%±10%, preferably 80%±5%, after eight hours in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising an active core and a coating applied to the active core wherein the active core comprises 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants, and wherein the coating comprises an osmotic membrane and a plasticizer.

In another embodiment, the present invention provides an oral dosage ECS single layer modified release tablet comprising 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein PF-06650833 is unmilled.

In another embodiment, the present invention provides an oral dosage ECS single layer modified release tablet comprising 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein dissolution of the tablet is 80%±10% after eight hours, preferably 80%±5% after eight hours.

In another embodiment, the present invention provides an oral dosage ECS single layer modified release tablet comprising 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein dissolution of the tablet is 80%±10% after eight hours, preferably 80%±5%, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein the tablet has NMT 0.05% total degradant products at 25° C./60% RH after 13 months.

In another embodiment, the present invention provides an oral dosage ECS single layer modified release tablet comprising 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein the tablet has NMT 0.05% total degradant products at 40° C./75% RH after 6 months.

In another embodiment, the present invention provides an oral dosage ECS single layer modified release tablet comprising 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 1-400 mgs 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer, wherein PF-06650833 is unmilled, wherein dissolution of the tablet is 80%±10%, preferably 80%±5%, after eight hours in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising an active core and a coating applied to the active core wherein the active core comprises 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate and wherein the coating comprises cellulose acetate and polyethylene glycol.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol, wherein PF-06650833 is unmilled.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol, wherein dissolution of the tablet is 80%±10% after eight hours, preferably 80%±5% after eight hours.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol, wherein dissolution of the tablet is 80%±5% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises comprising cellulose acetate and polyethylene glycol, wherein the tablet has NMT 0.05% total degradant products at 25° C./60% RH after 13 months.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol, wherein the tablet has NMT 0.05% total degradant products at 40° C./75% RH after 6 months.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 50-300 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein PF-06650833 is unmilled.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein dissolution of the tablet is 80%±10% after eight hours, preferably 80%±5% after eight hours.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release table comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein the tablet has NMT 0.05% total degradant products at 25° C./60% RH after 13 months.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein the tablet has NMT 0.05% total degradant products at 40° C./75% RH after 6 months.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 100-200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer.

In another embodiment, the present invention provides an oral dosage ECS single layer modified release tablet or capsule comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, dextrates, sodium chloride, hydroxyethylcellulose, colloidal silicon dioxide, copovidone, magnesium stearate, and sodium stearyl fumarate as an active core and a coating applied to the active core wherein the coating comprises cellulose acetate and polyethylene glycol.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein PF-06650833 is unmilled.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein dissolution of the tablet is 80%±10% after eight hours, preferably 80%±5% after eight hours.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release table comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein the tablet has NMT 0.05% total degradant products at 25° C./60% RH after 13 months.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein the tablet has NMT 0.05% total degradant products at 40° C./75% RH after 6 months.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 100 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 275-385 mgs of dextrates, 150-250 mgs of sodium chloride, 45-100 mgs of hydroxyethylcellulose, 1-5 mgs of colloidal silicon dioxide, 60-120 mgs of copovidone, 1-10 mgs of magnesium stearate, and 1-10 mgs of sodium stearyl fumarate as an active core and a coating applied to the active core wherein the coating comprises 10-45 mgs of cellulose acetate and 1-20 mgs of polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein PF-06650833 is unmilled.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein dissolution of the tablet is 80%±10% after eight hours, preferably 80%±5% after eight hours.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein the tablet has NMT 0.05% total degradant products at 25° C./60% RH after 13 months.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein the tablet has NMT 0.05% total degradant products at 40° C./75% RH after 6 months.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 7-15% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 35-50% dextrates, 22-32% sodium chloride, 4-12% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein PF-06650833 is unmilled.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein dissolution of the tablet is 80%±10% after eight hours, preferably 80%±5% after eight hours.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein the tablet has NMT 0.05% total degradant products at 25° C./60% RH after 13 months.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein the tablet has NMT 0.05% total degradant products at 40° C./75% RH after 6 months.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 9-13% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 40-47% dextrates, 25-30% sodium chloride, 6-10% hydroxyethylcellulose, 0.20-0.30% mgs of colloidal silicon dioxide, 7-11% copovidone, 0.35-0.65% magnesium stearate, and 0.35-0.65% of sodium stearyl fumarate as an active core, and a coating applied to the active core wherein the coating comprises 76-80% cellulose acetate and 20-24% polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein PF-06650833 is unmilled.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein dissolution of the tablet is 80%±10% after eight hours, preferably 80%±5% after eight hours.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein the tablet has NMT 0.05% total degradant products at 25° C./60% RH after 13 months.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein the tablet has NMT 0.05% total degradant products at 40° C./75% RH after 6 months.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 11.11% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 43.61% dextrates, 27.03% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol.

In another embodiment, the present invention provides an oral dosage ECS single layer modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein PF-06650833 is unmilled.

In another embodiment, the present invention provides an oral dosage ECS single layer modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein dissolution of the tablet is 80%±10% after eight hours, preferably 80%±5% after eight hours.

In another embodiment, the present invention provides an oral dosage ECS single layer modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein the tablet has NMT 0.05% total degradant products at 25° C./60% RH after 13 months.

In another embodiment, the present invention provides an oral dosage ECS single layer modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein the tablet has NMT 0.05% total degradant products at 40° C./75% RH after 6 months.

In another embodiment, the present invention provides an oral dosage ECS modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 175-225 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 310-350 mgs of dextrates, 185-225 mgs of sodium chloride, 66-80 mgs of hydroxyethylcellulose, 1-4 mgs of colloidal silicon dioxide, 74-88 mgs of copovidone, 2-7.5 mgs of magnesium stearate, and 2-7.5 mgs of sodium stearyl fumarate as an active core, a coating applied to the active core wherein the coating comprises 21-35 mgs of cellulose acetate and 5-14 mgs of polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol.

In another embodiment, the present invention provides an oral dosage ECS single layer modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein PF-06650833 is unmilled.

In another embodiment, the present invention provides an oral dosage ECS single layer modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein dissolution of the tablet is 80%±10% after eight hours, preferably 80%±5% after eight hours.

In another embodiment, the present invention provides an oral dosage ECS single layer modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein the tablet has NMT 0.05% total degradant products at 25° C./60% RH after 13 months.

In another embodiment, the present invention provides an oral dosage ECS single layer modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein the tablet has NMT 0.05% total degradant products at 40° C./75% RH after 6 months.

In another embodiment, the present invention provides an oral dosage ECS modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 190-210 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 320-340 mgs of dextrates, 195-215 mgs of sodium chloride, 70-74 mgs of hydroxyethylcellulose, 2-2.5 mgs of colloidal silicon dioxide, 79-83 mgs of copovidone, 4-5 mgs of magnesium stearate, and 4-5 mgs of sodium stearyl fumarate as an active core, a coating applied to the active core wherein the coating comprises 26-30 mgs of cellulose acetate and 7-9 mgs of polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs.

In another embodiment, the present invention provides an oral dosage ECS single layer modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein PF-06650833 is unmilled.

In another embodiment, the present invention provides an oral dosage ECS single layer modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein dissolution of the tablet is 80%±10% after eight hours, preferably 80%±5% after eight hours.

In another embodiment, the present invention provides an oral dosage ECS single layer modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein the tablet has NMT 0.05% total degradant products at 25° C./60% RH after 13 months.

In another embodiment, the present invention provides an oral dosage ECS single layer modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein the tablet has NMT 0.05% total degradant products at 40° C./75% RH after 6 months.

In another embodiment, the present invention provides an oral dosage ECS modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 200 mgs of 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 330.75 mgs of dextrates, 205 mgs of sodium chloride, 72 mgs of hydroxyethylcellulose, 2.25 mgs of colloidal silicon dioxide, 81 mgs of copovidone, 4.5 mgs of magnesium stearate, and 4.5 mgs of sodium stearyl fumarate as an active core, a coating applied to the active core wherein the coating comprises 28.86 mgs of cellulose acetate and 8.14 mgs of polyethylene glycol, and a total tablet weight of 937.00 mgs, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein PF-06650833 is unmilled.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein dissolution of the tablet is 80%±10% after eight hours, preferably 80%±5% after eight hours.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein the tablet has NMT 0.05% total degradant products at 25° C./60% RH after 13 months.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein the tablet has NMT 0.05% total degradant products at 40° C./75% RH after 6 months.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 17-28% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 31-42% dextrates, 17-28% sodium chloride, 5-11% hydroxyethylcellulose, 0.10-0.40% mgs of colloidal silicon dioxide, 5-13% copovidone, 0.25-0.75% magnesium stearate, and 0.25-0.75% of sodium stearyl fumarate as an active core, and a coating applied to the active core wherein the coating comprises 73-83% cellulose acetate and 17-27% polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein PF-06650833 is unmilled.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein dissolution of the tablet is 80%±10% after eight hours, preferably 80%±5% after eight hours.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein the tablet has NMT 0.05% total degradant products at 25° C./60% RH after 13 months.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein the tablet has NMT 0.05% total degradant products at 40° C./75% RH after 6 months.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein PF-06650833 is unmilled.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein dissolution of the tablet is 80%±10% after eight hours, preferably 80%±5% after eight hours.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein the tablet has NMT 0.05% total degradant products at 25° C./60% RH after 13 months.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C. and wherein the tablet has NMT 0.05% total degradant products at 25° C./60% RH after 13 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein the tablet has NMT 0.05% total degradant products at 40° C./75% RH after 6 months.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides an oral dosage ECS single layer modified release tablet comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein PF-06650833 is unmilled, wherein dissolution of the tablet is 80%±10% after eight hours, preferably 80%±5% after eight hours, in an aqueous medium comprising 50 nM sodium dihydrogen phosphate monohydrate and 0.25% sodium dodecyl sulfate at pH 6.8 at 37° C.±0.5° C., and wherein the tablet has NMT 0.05% total degradant products at 40° C./75% RH after 6 months by UPLC comprising an ACE Excel 2 C4 2.1×150 mm 2 μm column, a mobile phase of 0.1% perchloric acid in acetonitrile, a 46 minutes run time, and an UV absorbance detector at 210 nm.

In another embodiment, the present invention provides a method of treating hidradenitis suppurativa in a patient comprising administering orally to the patient in need of such treatment a therapeutically effective amount of 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof.

In another embodiment, the present invention provides a method of treating or preventing hidradenitis suppurativa in a patient comprising administering orally to the patient in need of such treatment one 100 mg MR-FORM2 tablet once daily.

In another embodiment, the present invention provides a method of treating or preventing hidradenitis suppurativa in a patient comprising administering orally to the patient in need of such treatment one 200 mg MR-FORM2 tablet once daily.

In another embodiment, the present invention provides a method of treating or preventing hidradenitis suppurativa in a patient comprising orally administering to the patient in need of such treatment one 100 mg MR-FORM2 tablet and one 200 mg MR-FORM2 tablet simultaneously or in sequence once daily.

In another embodiment, the present invention provides a method of treating or preventing hidradenitis suppurativa in a patient comprising orally administering to the patient in need of such treatment two 200 mg MR-FORM2 tablets simultaneously or in sequence once daily.

In another embodiment, the present invention provides a method of treating or preventing hidradenitis suppurativa in a patient comprising orally administering to the patient in need of such treatment one or more 20 mg MR-FORM1 tablets and one 100 mg MR-FORM2 tablet simultaneously or in sequence once daily.

In another embodiment, the present invention provides a method of treating or preventing hidradenitis suppurativa in a patient comprising orally administering to the patient in need of such treatment one or more 20 mg MR-FORM1 tablets and one 200 mg MR-FORM2 tablet simultaneously or in sequence once daily.

In another embodiment, the present invention provides a method of treating or preventing hidradenitis suppurativa in a patient comprising orally administering to the patient in need of such treatment one or more 20 mg MR-FORM1 tablets, one 100 mg MR-FORM2 tablet, and one 200 mg MR-FORM2 tablet simultaneously or in sequence once daily.

In another embodiment, the present invention provides a method of treating or preventing hidradenitis suppurativa in a patient comprising orally administering to the patient in need of such treatment one or more 20 mg MR-FORM1 tablets, and two 200 mg MR-FORM2 tablets simultaneously or in sequence once daily.

In another embodiment, the present invention provides a method of treating or preventing hidradenitis suppurativa in a patient comprising administering orally to the patient in need of such treatment one 100 mg MR-FORM3 tablet once daily.

In another embodiment, the present invention provides a method of treating or preventing hidradenitis suppurativa in a patient comprising administering orally to the patient in need of such treatment one 200 mg MR-FORM3 tablet once daily.

In another embodiment, the present invention provides a method of treating or preventing hidradenitis suppurativa in a patient comprising orally administering to the patient in need of such treatment one 100 mg MR-FORM3 tablet and one 200 mg MR-FORM3 tablet simultaneously or in sequence once daily.

In another embodiment, the present invention provides a method of treating or preventing hidradenitis suppurativa in a patient comprising orally administering to the patient in need of such treatment two 200 mg MR-FORM3 tablets simultaneously or in sequence once daily.

In another embodiment, the present invention provides a method of treating or preventing hidradenitis suppurativa in a patient comprising orally administering to the patient in need of such treatment one or more 20 mg MR-FORM1 tablets and one 100 mg MR-FORM3 tablet simultaneously or in sequence once daily.

In another embodiment, the present invention provides a method of treating or preventing hidradenitis suppurativa in a patient comprising orally administering to the patient in need of such treatment one or more 20 mg MR-FORM1 tablets and one 200 mg MR-FORM3 tablet simultaneously or in sequence once daily.

In another embodiment, the present invention provides a method of treating or preventing hidradenitis suppurativa in a patient comprising orally administering to the patient in need of such treatment one or more 20 mg MR-FORM1 tablets, one 100 mg MR-FORM3 tablet, and one 200 mg MR-FORM3 tablet simultaneously or in sequence once daily.

In another embodiment, the present invention provides a method of treating or preventing hidradenitis suppurativa in a patient comprising orally administering to the patient in need of such treatment one or more 20 mg MR-FORM1 tablets, and two 200 mg MR-FORM3 tablets simultaneously or in sequence once daily.

The present invention is also related to treating immune, autoimmune, and inflammatory disease such as inflammatory bowel disease, ulcerative colitis, Crohn's disease, non-alcoholic steatohepatitis (NASH), liver fibrosis, non-alcoholic fatty liver disease (NAFLD), iodopathic pulmonary fibrosis (IPF), rheumatoid arthritis (RA), atopic dermatitis, psoriasis, psoriatic arthritis, stasis dermatitis, lupus, ankylosing spondylitis, alopecia, vitiligo, and hidradenitis suppurativa (HS). The combinations of the present invention for treating such diseases include: 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof; and 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof. In particular, rheumatoid arthritis may be treated with the combination therapies of the present invention.

Rheumatoid arthritis is characterized by dysregulation of the innate and adaptive immune systems with a common clinical phenotype arising from diverse pathways in individual patients. The pre-RA phase lasts months to years and is characterized by the presence of circulating autoantibodies, increased concentration and range of inflammatory cytokines and chemokines, and altered metabolism. Eventually RA patients develop synovitis characterized by frank inflammation, stromal compartment changes and tissue modification resulting in articular damage.

Recent data suggests that autoantibodies generated by the adaptive immune system against post-translationally modified proteins commonly found in pre-RA patients eventually expand their capabilities to recognize osteoclasts. One consequence of the autoantibody-osteoclast interaction is the induction of pain and release of IL-8 providing a route to subsequent leukocyte recruitment and articular inflammation. Recruitment of new innate and adaptive immune cells promotes stromal cell activation leading to the production of additional cytokines and chemokines creating a positive feedback loop and a self-perpetuating process with inadequate negative regulators required for termination. Currently, there is no cure for RA. (Firestein and McInnes, Immunity, 2017 Feb. 21, 46(2), 183-196).

In another embodiment, the present invention provides a pharmaceutical combination comprising one 1-400 mg tablet or capsule of 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and one 1-200 mg tablet or capsule of 1-((2S,5R)-5-((7H-pyrrolo[2,3-d]pyrimidin-4-yl)amino)-2-methylpiperidin-1-yl)prop-2-en-1-one (PF-06651600), or an equivalent amount of PF-06651600 in the form of a pharmaceutically acceptable salt thereof.

In another embodiment, the present invention provides a pharmaceutical combination comprising one 100-300 mg tablet or capsule of 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and one 50-150 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof.

In another embodiment, the present invention provides a pharmaceutical combination comprising one 200 mg MR-FORM2 tablet and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof.

In another embodiment, the present invention provides a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof.

In another embodiment, the present invention provides a pharmaceutical combination comprising one 200 mg MR-FORM3 tablet and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof.

In another embodiment, the present invention provides a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof.

In another embodiment, the present invention provides a method of treating or preventing an immune, autoimmune, or inflammatory disease in a patient comprising administering orally to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence once daily.

In another embodiment, the present invention provides a method of treating or preventing inflammatory bowel disease, ulcerative colitis, Crohn's disease, non-alcoholic steatohepatitis (NASH), liver fibrosis, non-alcoholic fatty liver disease (NAFLD), iodopathic pulmonary fibrosis (IPF), rheumatoid arthritis (RA), atopic dermatitis, psoriasis, psoriatic arthritis, stasis dermatitis, lupus, ankylosing spondylitis, alopecia, vitiligo, or hidradenitis suppurativa (HS) in a patient comprising administering orally to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence once daily.

In another embodiment, the present invention provides a method of treating or preventing rheumatoid arthritis in a patient comprising administering orally to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM2 tablet and one 0.5-200 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM2 tablet and one 0.5-200 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 0.5-200 mgs tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM2 tablet and one 50-150 mgs tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM2 tablet and one 50-150 mgs tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 50-150 mgs tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM2 tablet and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM2 tablet and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet of 1-((2S,5R)-5-((7H-pyrrolo[2,3-d]pyrimidin-4-yl)amino)-2-methylpiperidin-1-yl)prop-2-en-1-one simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM3 tablet and one 0.5-200 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM3 tablet and one 0.5-200 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 0.5-200 mgs tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM3 tablet and one 50-150 mgs tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM3 tablet and one 50-150 mgs tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 50-150 mgs tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM3 tablet and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM3 tablet and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet of 1-((2S,5R)-5-((7H-pyrrolo[2,3-d]pyrimidin-4-yl)amino)-2-methylpiperidin-1-yl)prop-2-en-1-one simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein inflammatory activities from the innate and adaptive immune systems are reduced.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein monocyte and B cell levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein TNFα levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein TNFα and IL-17F levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein TNFα and IL-17F levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein TNFα and IL-6 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein TNFα and IL-6 levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin and IL-6 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin and IL-6 levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin and IL-17F levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin and IL-17F levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8 and IL-6 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8 and IL-6 levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8 and IL-17F levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8 and IL-17F levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin, TNFα, and IL-6 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin, TNFα, and IL-6 levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin, TNFα, and IL-17F levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin, TNFα, and IL-17F levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin, TNFα, and IL-6 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin, TNFα, and IL-6 levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin, TNFα, and IL-17F levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein E-selectin, TNFα, and IL-17F levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, TNFα, and IL-6 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, TNFα, and IL-6 levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, TNFα, and IL-17F levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, TNFα, and IL-17F levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, TNFα, and IL-6 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, TNFα, and IL-6 levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, TNFα, and IL-17F levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, TNFα, and IL-17F levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-17F, IL-6, E-selectin, IL-8, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-17F, IL-6, E-selectin, IL-8, and TNFα levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-17F, IL-6, E-selectin, IL-8, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-17F, IL-6, E-selectin, IL-8, and TNFα levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein one of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein two of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein three of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein four of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein five of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein six of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein seven of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein eight of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein nine of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein ten of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein eleven of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein twelve of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein one of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein two of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein three of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein four of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein five of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein six of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein seven of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein eight of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein nine of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein ten of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein eleven of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein twelve of IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 100 mg tablet or capsule of 1-((2S,5R)-5-((7H-pyrrolo[2,3-d]pyrimidin-4-yl)amino)-2-methylpiperidin-1-yl)prop-2-en-1-one, or an equivalent amount of PF-06651600 in the form of a pharmaceutically acceptable salt thereof, simultaneously or in sequence once daily wherein IL-8, IL-17A, IL-17F, IL-6, PAI-I, MCP-1, VCAM-1, CD40, E-selectin, CD69, IL-1α, PGE₂, and TNFα levels are reduced at the inflammation site.

In another embodiment, the present invention provides a pharmaceutical combination comprising one 1-400 mg tablet or capsule of 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and one 1-20 mg tablet or capsule of tofacitinib, or an equivalent amount of tofacitinib in the form of a pharmaceutically acceptable salt thereof.

In another embodiment, the present invention provides a pharmaceutical combination comprising one 100-300 mg tablet or capsule of 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and one 5-11 mg tablet or capsule of tofacitinib, or an equivalent amount of tofacitinib in the form of a pharmaceutically acceptable salt thereof.

In another embodiment, the present invention provides a pharmaceutical combination comprising one 200 mg MR-FORM2 tablet and one 11 mg tablet or capsule of tofacitinib, or an equivalent amount of tofacitinib in the form of a pharmaceutically acceptable salt thereof.

In another embodiment, the present invention provides a pharmaceutical combination comprising two 200 mg MR-FORM2 tablets and one 11 mg tablet or capsule of tofacitinib, or an equivalent amount of tofacitinib in the form of a pharmaceutically acceptable salt thereof.

In another embodiment, the present invention provides a pharmaceutical combination comprising two 200 mg MR-FORM2 tablets and one 11 mg tablet of tofacitinib.

In another embodiment, the present invention provides a pharmaceutical combination comprising two 200 mg MR-FORM2 tablets and one 11 mg extended release tablet of tofacitinib wherein tofacitinib is a salt of citric acid.

In another embodiment, the present invention provides a pharmaceutical combination comprising one 200 mg MR-FORM3 tablet and one 11 mg tablet or capsule of tofacitinib, or an equivalent amount of tofacitinib in the form of a pharmaceutically acceptable salt thereof.

In another embodiment, the present invention provides a pharmaceutical combination comprising two 200 mg MR-FORM3 tablets and one 11 mg tablet or capsule of tofacitinib, or an equivalent amount of tofacitinib in the form of a pharmaceutically acceptable salt thereof.

In another embodiment, the present invention provides a pharmaceutical combination comprising two 200 mg MR-FORM3 tablets and one 11 mg tablet of tofacitinib.

In another embodiment, the present invention provides a pharmaceutical combination comprising two 200 mg MR-FORM3 tablets and one 11 mg extended release tablet of tofacitinib wherein tofacitinib is a salt of citric acid.

In another embodiment, the present invention provides a method of treating or preventing an immune, autoimmune, or inflammatory disease in a patient comprising administering orally to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and tofacitinib, or an equivalent amount of tofacitinib in the form of a pharmaceutically acceptable salt thereof, simultaneously or in sequence once daily.

In another embodiment, the present invention provides a method of treating or preventing inflammatory bowel disease, ulcerative colitis, Crohn's disease, non-alcoholic steatohepatitis (NASH), liver fibrosis, non-alcoholic fatty liver disease (NAFLD), iodopathic pulmonary fibrosis (IPF), rheumatoid arthritis (RA), atopic dermatitis, psoriasis, psoriatic arthritis, stasis dermatitis, lupus, ankylosing spondylitis, alopecia, vitiligo, or hidradenitis suppurativa (HS) in a patient comprising administering orally to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or an equivalent amount of PF-06650833 in the form of a pharmaceutically acceptable salt thereof, and tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM2 tablet and one 1-22 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM2 tablet and one 1-22 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 1-22 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM2 tablet and one 5-11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM2 tablet and one 5-11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 5-11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM2 tablet and one 11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM2 tablet and one 11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM2 tablet and one 11 mg extended release tablet of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM2 tablet and one 11 mg extended release tablet of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib wherein tofacitinib is the citrate salt, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM3 tablet and one 1-22 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM3 tablet and one 1-22 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 1-22 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM3 tablet and one 5-11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM3 tablet and one 5-11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 5-11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM3 tablet and one 11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM3 tablet and one 11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg tablet or capsule of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 100 mg MR-FORM3 tablet and one 11 mg extended release tablet of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising one 200 mg MR-FORM3 tablet and one 11 mg extended release tablet of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib wherein tofacitinib is the citrate salt, simultaneously or in sequence once daily.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein inflammatory activities from the innate and adaptive immune systems are reduced.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein monocyte and B cell levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein IL-8 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein VCAM-1 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein MIG levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein neutrophil levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein inflammatory activities from the innate and adaptive immune systems are reduced.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein monocyte and B cell levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein IL-8 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein VCAM-1 levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein MIG levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein neutrophil levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein two of IL-8, VCAM-1, MIG, and neutrophil levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein three of IL-8, VCAM-1, MIG, and neutrophil levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein IL-8, VCAM-1, MIG, and neutrophil levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein TNFα levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein two of IL-8, VCAM-1, MIG, and neutrophil levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein three of IL-8, VCAM-1, MIG, and neutrophil levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein IL-8, VCAM-1, MIG, and neutrophil levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein TNFα levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein TNFα levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein two of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein two of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein two of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein two of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein three of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein three of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein four of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein four of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein five of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein five of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein six of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein six of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein seven of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein seven of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM2 tablets and one 11 mg extended release tablet of tofacitinib, or an equivalent amount of tofacitinib in the form of a pharmaceutically acceptable salt thereof, simultaneously or in sequence once daily, wherein IL-6, MCP-1, VCAM-1, CD40, E-selectin, CD69, IL-8, IL-1α, IL-17F, PGE₂, TNFα, Eto3, P-selectin, CD38, IL-2, MIG, ITAC, monocyte, eosinphil, basophil, B cell and T cell levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein three of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein three of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein four of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein four of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein five of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein five of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein six of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein six of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein seven of E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein seven of E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein E-selectin, IL-8, TNFα, Eto3, VCAM-1, P-selectin, IL-17F, and IL-6, levels are reduced at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein E-selectin, IL-8, TNFα, Eto3, VCAM-1, P-selectin, IL-17F, and IL-6, levels are reduced by 50% or more at the inflammation site.

In another embodiment, the present invention provides a method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, or an equivalent amount of tofacitinib in the form of a pharmaceutically acceptable salt thereof, simultaneously or in sequence once daily, wherein IL-6, MCP-1, VCAM-1, CD40, E-selectin, CD69, IL-8, IL-1α, IL-17F, PGE₂, TNFα, Eto3, P-selectin, CD38, IL-2, MIG, ITAC, monocyte, eosinphil, basophil, B cell and T cell levels are reduced at the inflammation site.

Protein kinases are families of enzymes that catalyze the phosphorylation of specific residues in proteins, broadly classified in tyrosine and serine/threonine kinases. Inappropriate activity arising from dysregulation of certain kinases by a variety of mechanisms is believed to underlie the causes of many diseases, including but not limited to, cancer, cardiovascular diseases, allergies, asthma, respiratory diseases, autoimmune diseases, inflammatory diseases, bone diseases, metabolic diseases, and neurological and neurodegenerative diseases. As such, potent and selective inhibitors of kinases are sought as potential treatments for a variety of human diseases.

There is considerable interest in targeting the innate immune system in the treatment of immune, autoimmune and inflammatory diseases. Receptors of the innate immune system provide the first line of defense against bacterial and viral insults. These receptors recognize bacterial and viral products as well as pro-inflammatory cytokines and thereby initiate a signaling cascade that ultimately results in the up-regulation of inflammatory cytokines such as TNFα, IL6, and interferons. Recently it has become apparent that self-generated ligands such as nucleic acids and products of inflammation such as high-mobility group protein B1 (HMGB1) and Advanced Glycated End-products (AGE) are ligands for Toll-like receptors (TLRs) which are key receptors of the innate immune system (O'Neill 2003, Kanzler et al 2007, Wagner 2006). This demonstrates the role of TLRs in the initiation and perpetuation of inflammation due to autoimmunity.

Interleukin-1 receptor associated kinase 4 (IRAK4) is a ubiquitously expressed serine/threonine kinase involved in the regulation of innate immunity (Suzuki & Saito 2006). IRAK4 is responsible for initiating signaling from TLRs and members of the IL-1/18 receptor family. Kinase-inactive knock-ins and targeted deletions of IRAK4 in mice were reported to cause reductions in TLR and IL-1 induced pro-inflammatory cytokines (Kawagoe et al 2007; Fraczek et al. 2008; Kim et al. 2007). IRAK4 kinase-dead knock-in mice have also been shown to be resistant to induced joint inflammation in the antigen-induced-arthritis (AIA) and serum transfer-induced (K/BxN) arthritis models (Koziczak-Holbro 2009). Likewise, humans deficient in IRAK4 also appear to display the inability to respond to challenge by Toll ligands and IL-1 (Hernandez & Bastian 2006). However, the immunodeficient phenotype of IRAK4-null individuals is narrowly restricted to challenge by gram positive bacteria, but not gram negative bacteria, viruses or fungi. This gram positive sensitivity also lessens with age, implying redundant or compensating mechanisms for innate immunity in the absence of IRAK4 (Lavine et al 2007).

These data indicate that inhibitors of IRAK4 kinase activity may have therapeutic value in treating cytokine driven immune, autoimmune, and inflammatory diseases while having minimal immunosuppressive side effects. Additional recent studies suggest that targeting IRAK4 may be useful in other inflammatory pathologies such as atherosclerosis and diffuse large B-cell lymphoma (Rekhter et al 2008; Ngo et al 2011). Therefore, inhibitors of IRAK4 kinase activity are potential therapeutics for a wide variety of diseases including but not limited to autoimmunity, inflammation, cardiovascular diseases, cancer, and metabolic diseases. See the following references for additional information: N. Suzuki and T. Saito, Trends in Immunology, 2006, 27, 566. T. Kawagoe, S. Sato, A. Jung, M. Yamamoto, K. Matsui, H. Kato, S. Uematsu, O. Takeuchi and S. Akira, Journal of Experimental Medicine, 2007, 204, 1013. J. Fraczek, T. W. Kim, H. Xiao, J. Yao, Q. Wen, Y. Li, J.-L. Casanova, J. Pryjma and X. Li, Journal of Biological Chemistry, 2008, 283, 31697. T. W. Kim, K. Staschke, K. Bulek, J. Yao, K. Peters, K.-H. Oh, Y. Vandenburg, H. Xiao, W. Qian, T. Hamilton, B. Min, G. Sen, R. Gilmour and X. Li, Journal of Experimental Medicine, 2007, 204, 1025. M. Koziczak-Holbro, A. Littlewood-Evans, B. Pollinger, J. Kovarik, J. Dawson, G. Zenke, C. Burkhart, M. Muller and H. Gram, Arthritis & Rheumatism, 2009, 60, 1661. M. Hernandez and J. F. Bastian, Current Allergy and Asthma Reports, 2006, 6, 468. E. Lavine, R. Somech, J. Y. Zhang, A. Puel, X. Bossuyt, C. Picard, J. L. Casanova and C. M. Roifman, Journal of Allergy and Clinical Immunology, 2007, 120, 948. M. Rekhter, K. Staschke, T. Estridge, P. Rutherford, N. Jackson, D. Gifford-Moore, P. Foxworthy, C. Reidy, X.-d. Huang, M. Kalbfleisch, K. Hui, M.-S. Kuo, R. Gilmour and C. J. Vlahos, Biochemical and Biophysical Research Communications, 2008, 367, 642. O'Neill, L. A. (2003). “Therapeutic targeting of Toll-like receptors for inflammatory and infectious diseases.” Curr Opin Pharmacol 3(4): 396. Kanzler, H et al. (2007) “Therapeutic targeting of innate immunity with toll-like receptor agonists and antagonists.” Nature Medicine 13:552. Wagner, H. (2006) “Endogenous TLR ligands and autoimmunity” Advances in Immunol 91: 159. Ngo, V. N. et al. (2011) “Oncogenically active MyD88 mutations in human lymphoma” Nature 470: 115.

In another embodiment, the present invention provides a method for treating or preventing a neurodegenerative or neuroinflammatory disease such as multiple sclerosis, amyotropic lateral sclerosis, Guillain-Barre disease, autoimmune encephalomyelitis, Alzheimer's disease, major depressive disease, traumatic brain injury, epilepsy, Parkinson's disease, or bipolar disease in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing a neurodegenerative or neuroinflammatory disease such as multiple sclerosis, amyotropic lateral sclerosis, Guillain-Barre disease, autoimmune encephalomyelitis, Alzheimer's disease, major depressive disease, traumatic brain injury, epilepsy, Parkinson's disease, or bipolar disease in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing juvenile arthritis, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile idiopathic arthritis (JIA), juvenile psoriatic arthritis, giant cell arteritis, or secondary osteoarthritis from inflammatory diseases in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing juvenile arthritis, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile idiopathic arthritis (JIA), juvenile psoriatic arthritis, giant cell arteritis, or secondary osteoarthritis from inflammatory diseases in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing systemic lupus erythematosus, juvenile systemic lupus erythematosus, lupus nephritis, Sjögren's syndrome, scleroderma (systemic sclerosis), Raynaud's phenomenon, juvenile scleroderma, polymyositis, dermatomyositis, polymyositis-dermatomyositis, mixed connective tissue disease, sarcoidosis, fibromyalgia, vasculitis microscopic polyangiitis, vasculitis, eosinophilic granulomatosis with polyangiitis (formerly known as Churg-Strauss syndrome), granulomatosis with polyangiitis (formerly known as Wegener's granulomatosis), polyarteritis nodosa, Henoch-Schönlein purpura, idiopathic thrombocytopenic thrombotic purpura, juvenile vasculitis, polyarteritis nodossa (also known as panarteritis nodosa, periarteritis nodosa, Kussmaul disease, Kussmaul-Maier disease or PAN), serum sickness, Myasthenia gravis, Takayasu's arteritis, Behçet's syndrome, Kawasaki's disease (mucocutaneous lymph node syndrome), Buerger's disease (thromboangiitis obliterans), Vogt-Koyanagi-Harada syndrome, Addison's disease, Hashimoto's thyroiditis, sclerosing cholangitis, membranous glomerulopathy, polymyositis, myositis, atherosclerosis, autoimmune hemolytic anemia, autoimmune orchitis, or Goodpasture's disease in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing systemic lupus erythematosus, juvenile systemic lupus erythematosus, lupus nephritis, Sjögren's syndrome, scleroderma (systemic sclerosis), Raynaud's phenomenon, juvenile scleroderma, polymyositis, dermatomyositis, polymyositis-dermatomyositis, mixed connective tissue disease, sarcoidosis, fibromyalgia, vasculitis microscopic polyangiitis, vasculitis, eosinophilic granulomatosis with polyangiitis (formerly known as Churg-Strauss syndrome), granulomatosis with polyangiitis (formerly known as Wegener's granulomatosis), polyarteritis nodosa, Henoch-Schönlein purpura, idiopathic thrombocytopenic thrombotic purpura, juvenile vasculitis, polyarteritis nodossa (also known as panarteritis nodosa, periarteritis nodosa, Kussmaul disease, Kussmaul-Maier disease or PAN), serum sickness, Myasthenia gravis, Takayasu's arteritis, Behçet's syndrome, Kawasaki's disease (mucocutaneous lymph node syndrome), Buerger's disease (thromboangiitis obliterans), Vogt-Koyanagi-Harada syndrome, Addison's disease, Hashimoto's thyroiditis, sclerosing cholangitis, membranous glomerulopathy, polymyositis, myositis, atherosclerosis, autoimmune hemolytic anemia, autoimmune orchitis, or Goodpasture's disease in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing is celiac sprue, celiac diseases, proctitis, eosinophilic gastroenteritis, autoimmune atrophic gastritis of pernicious anemia, or mastocytosis in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing is celiac sprue, celiac diseases, proctitis, eosinophilic gastroenteritis, autoimmune atrophic gastritis of pernicious anemia, or mastocytosis in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing plaque psoriasis, Guttate psoriasis, psoriatic epidermal hyperplasia, inverse psoriasis, pustular psoriasis, erythrodermic psoriasis atopic dermatitis, eczema dermatitis, dermatitis, pruritus, autoimmune alopecia, epidermal hyperplasia, juvenile dermatomyositis, or dermatomyositis in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing plaque psoriasis, Guttate psoriasis, psoriatic epidermal hyperplasia, inverse psoriasis, pustular psoriasis, erythrodermic psoriasis atopic dermatitis, eczema dermatitis, dermatitis, pruritus, autoimmune alopecia, epidermal hyperplasia, juvenile dermatomyositis, or dermatomyositis in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing autoimmune hepatitis, chronic aggressive hepatitis, or primary biliary sclerosis in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing autoimmune hepatitis, chronic aggressive hepatitis, or primary biliary sclerosis in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing Graves' disease, noninfectious uveitis, dry eye syndrome, sympathetic ophthalmia, Cogan's syndrome, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, keratoconjunctivitis sicca (dry eye), phlyctenule, iridocyclitis, sarcoidosis, endocrine ophthalmopathy, sympathetic ophthalmitis, allergic conjunctivitis, ocular neovascularization, or proliferative diabetic retinopathy in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing Graves' disease, noninfectious uveitis, dry eye syndrome, sympathetic ophthalmia, Cogan's syndrome, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, keratoconjunctivitis sicca (dry eye), phlyctenule, iridocyclitis, sarcoidosis, endocrine ophthalmopathy, sympathetic ophthalmitis, allergic conjunctivitis, ocular neovascularization, or proliferative diabetic retinopathy in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing asthma, allergy, chronic obstructive pulmonary disease, or acute respiratory disease in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing asthma, allergy, chronic obstructive pulmonary disease, or acute respiratory disease in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing alimentary/gastrointestinal tract cancer, colon cancer, liver cancer, skin cancer including mast cell tumor and squamous cell carcinoma, breast and mammary cancer, ovarian cancer, prostate cancer, leukemia, acute myeloid leukemia, T cell acute lymphoblastic leukemia, or adult T cell leukemia, diffuse large B cell lymphoma, cutaneous T-cell lymphoma, non-Hodgkin lymphoma, kidney cancer, lung cancer, muscle cancer, bone cancer, bladder cancer, brain cancer, melanoma including oral and metastatic melanoma, Kaposi's sarcoma, multiple myeloma, myeloproliferative diseases, glioblastoma, oligodendroglioma, pancreatic cancer, brain tumors, or gliomas including astrocytoma in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing alimentary/gastrointestinal tract cancer, colon cancer, liver cancer, skin cancer including mast cell tumor and squamous cell carcinoma, breast and mammary cancer, ovarian cancer, prostate cancer, leukemia, acute myeloid leukemia, T cell acute lymphoblastic leukemia, or adult T cell leukemia, diffuse large B cell lymphoma, cutaneous T-cell lymphoma, non-Hodgkin lymphoma, kidney cancer, lung cancer, muscle cancer, bone cancer, bladder cancer, brain cancer, melanoma including oral and metastatic melanoma, Kaposi's sarcoma, multiple myeloma, myeloproliferative diseases, glioblastoma, oligodendroglioma, pancreatic cancer, brain tumors, or gliomas including astrocytoma in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

In another embodiment, the present invention provides a method for treating or preventing Type I diabetes mellitus, Type II diabetes mellitus, or Juvenile onset diabetes in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and 1-((2S,5R)-5-((7H-pyrrolo[2,3-d]pyrimidin-4-yl)amino)-2-methylpiperidin-1-yl)prop-2-en-1-one, or a pharmaceutically acceptable salt thereof, simultaneously or in sequence.

In another embodiment, the present invention provides a method for treating or preventing Type I diabetes mellitus, Type II diabetes mellitus, or Juvenile onset diabetes in a patient comprising administering to the patient in need of such treatment a therapeutically effective amount of a pharmaceutical combination comprising 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof, and tofacitinib, or a pharmaceutically acceptable salt thereof, simultaneously or in sequence.

### Definitions

The term “mean dissolution of the tablet,” as used herein means the average percentage of dissolved tablet, or tablet dissolution profile, for at least 20 tablets under certain conditions such as temperature, RH, and duration.

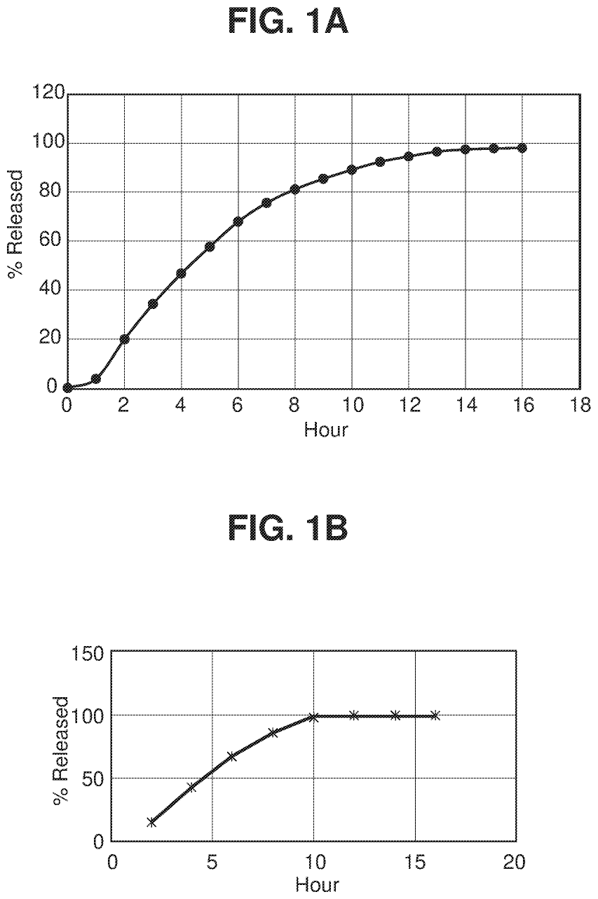

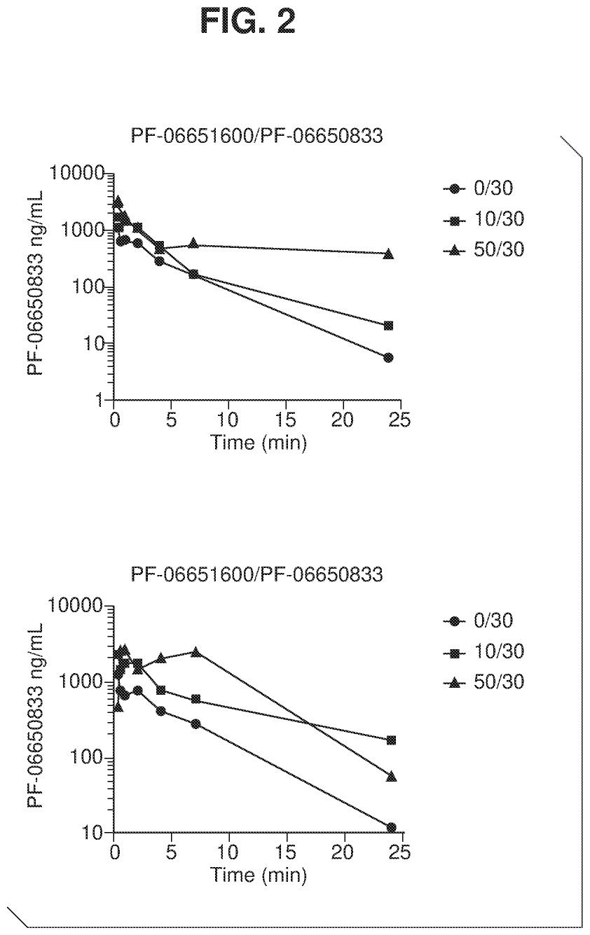

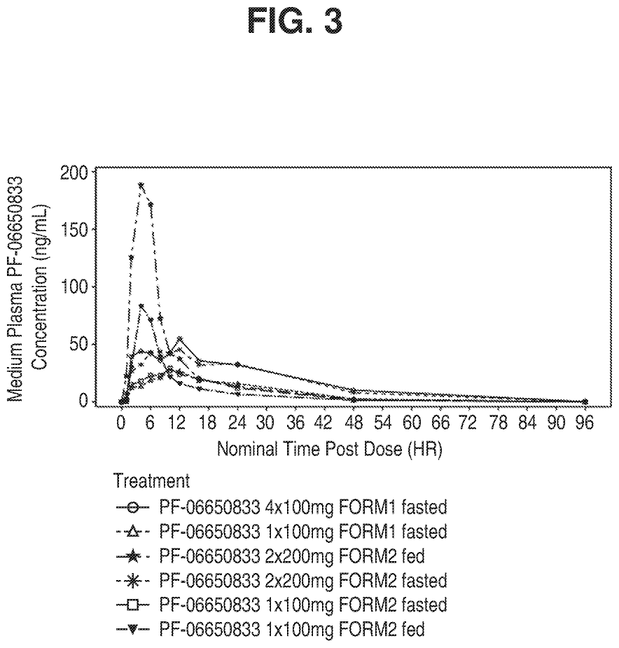

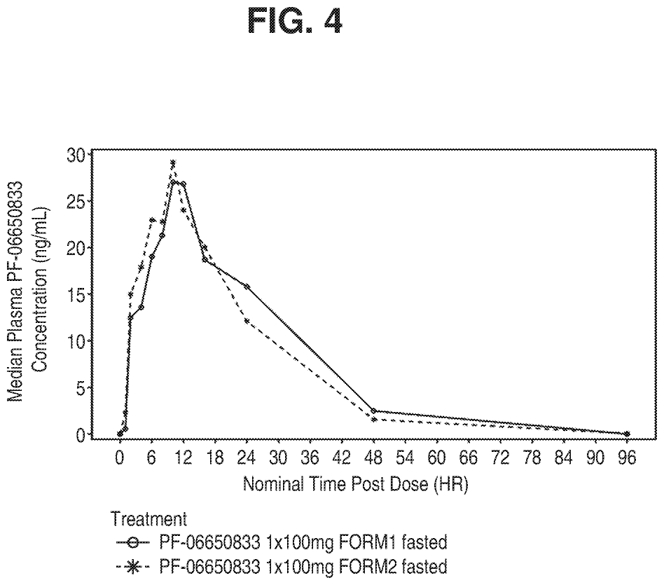

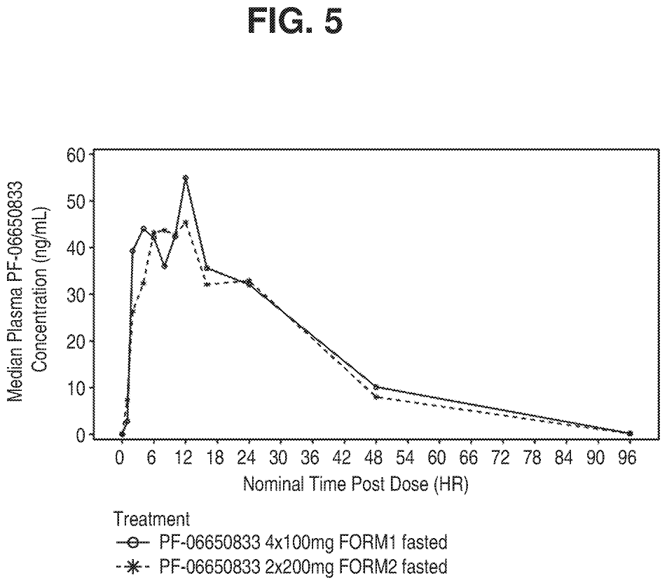

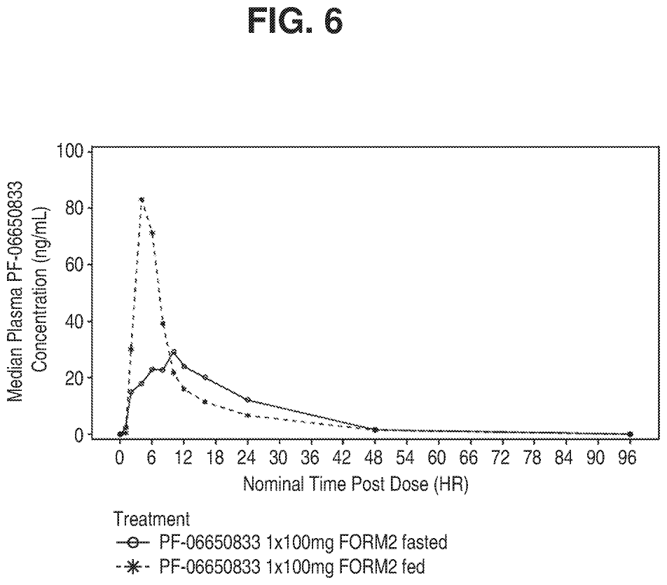

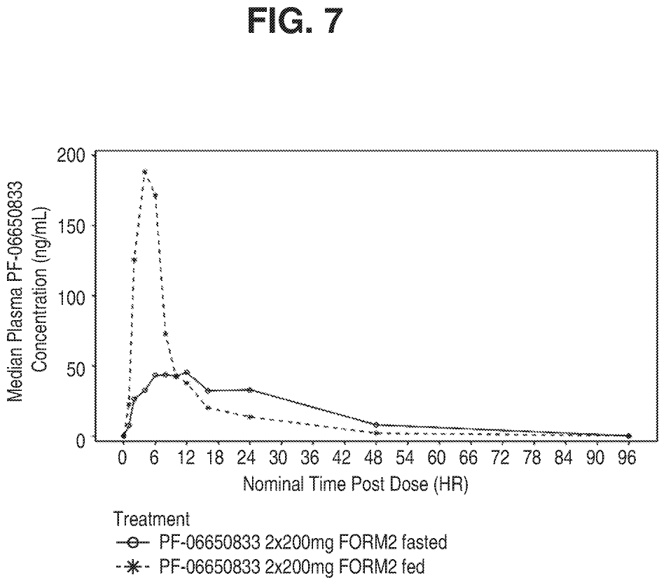

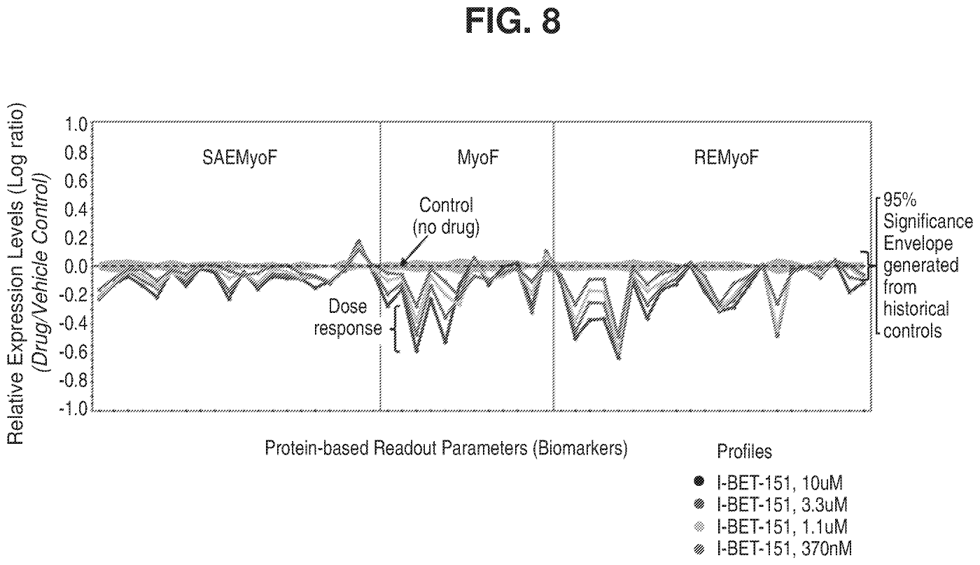

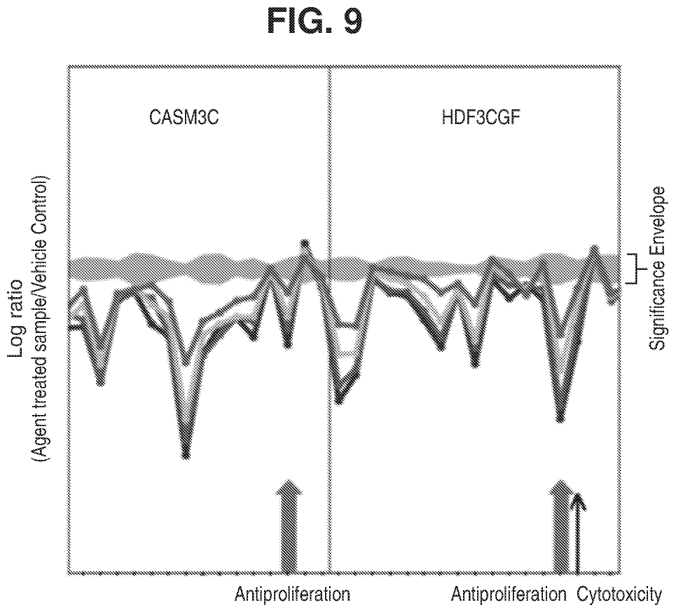

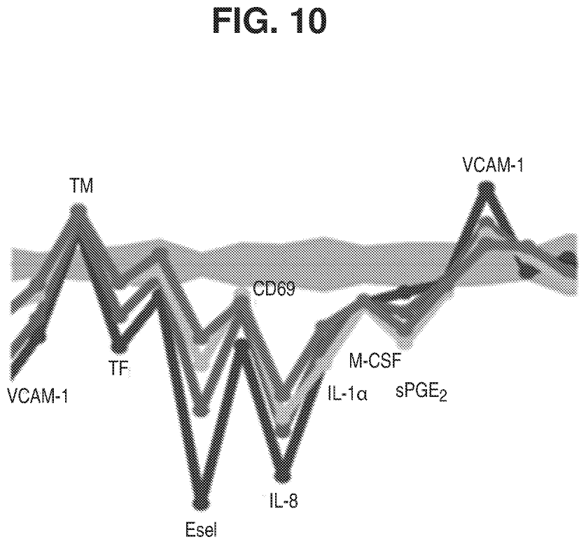

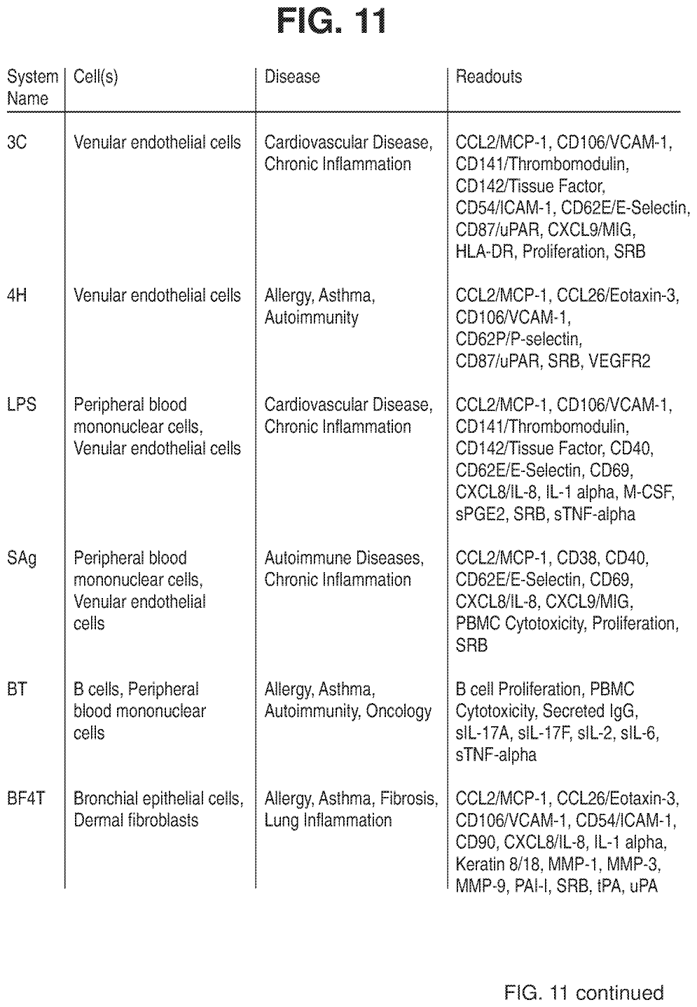

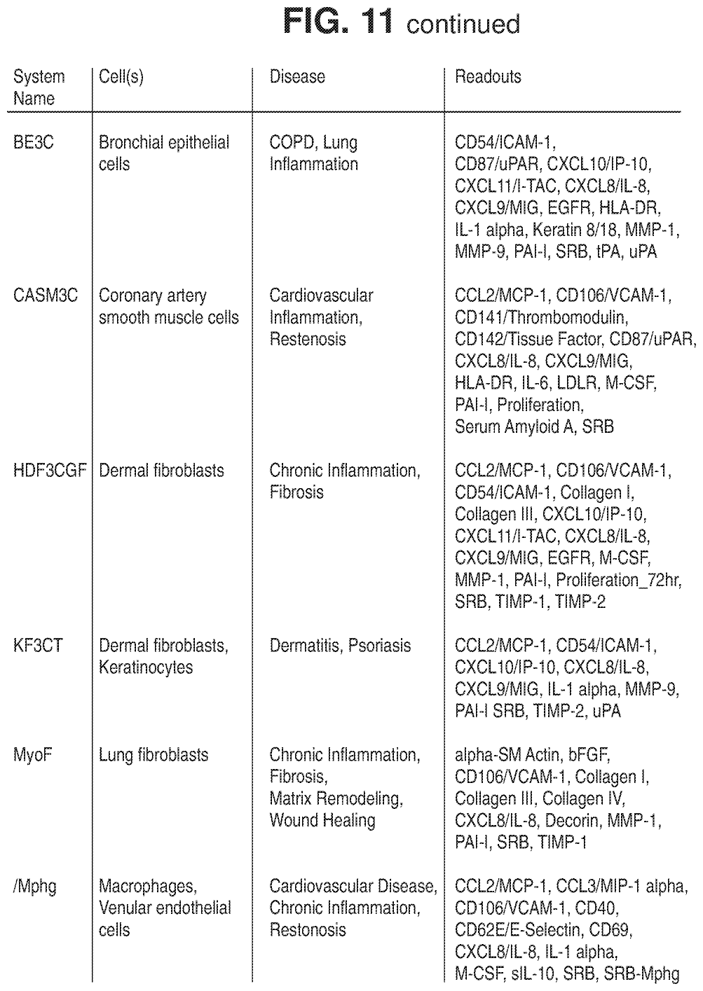

The term “modified release” or “controlled release” as used herein means a tablet dissolution profile similar to FIG. 1A wherein dissolution is 80%±10% after eight (8) hours, preferably 80%±5% after eight (8) hours. It is to be understood that the tablet manufacturing process may render individual tablets with individual dissolution profiles of 70% to 90% after eight (8) hours, preferably 75% to 85% after eight (8) hours.

The term “NMT,” means not-more-than.

The term “20 mg MR-FORM1,” as used herein, means a SCT bilayer modified release tablet comprising 20 mgs of 1-(((2S,3S,4S)-3-ethyl-4-fluoro-5-oxopyrrolidin-2-yl)methoxy)-7-methoxyisoquinoline-6-carboxamide, polyethylene oxide (200,000 molecular weight) and magnesium stearate as the active layer. Polyethylene oxide (5,000,000 molecular weight), sodium chloride, microcrystalline cellulose, magnesium stearate, and FD&C blue aluminum lake #2 comprise the sweller layer. Cellulose acetate and polyethylene glycol as the coating wherein acetone and purified water were used as solvents during processing.

The term “100 mg MR-FORM1,” as used herein, means a SCT bilayer modified release tablet comprising an active layer, a swellable layer, and a coating applied to the bilayer (i.e. active layer and swellable layer). The active layer comprises 100.00 mgs 1-(((2S,3S,4S)-3-ethyl-4-fluoro-5-oxopyrrolidin-2-yl)methoxy)-7-methoxyisoquinoline-6-carboxamide, 395.00 mgs polyethylene oxide (200,000 molecular weight), and 5.00 mgs magnesium stearate resulting in a total active layer weight of 500.00 mgs. The swellable layer comprises 138.21 mgs polyethylene oxide (5,000,000 molecular weight), 76.50 mgs sodium chloride, 38.25 mgs microcrystalline cellulose, 1.275 mgs magnesium stearate, and 0.765 mgs FD&C blue aluminum lake #2 resulting in a total swellable layer weight of 255.00 mgs. The coating surrounding the bilayer (i.e. active layer and swellable layer) comprises 39.00 mgs cellulose acetate and 11.00 mgs polyethylene glycol resulting in a total coating weight of 50.00 mgs and a total tablet weight of 805.00 mgs. It is to be understood that the coating covers nearly 100% of the bilayer except for the laser-drilled delivery port in the tablet that allows 1-(((2S,3S,4S)-3-ethyl-4-fluoro-5-oxopyrrolidin-2-yl)methoxy)-7-methoxyisoquinoline-6-carboxamide to exit the tablet under the appropriate physiological conditions.

The term “100 mg MR-FORM2,” as used herein, means an ECS single layer modified release tablet comprising an active core and a coating applied to the active core, wherein the active core comprises 100.000 mgs 1-(((2S,3S,4S)-3-ethyl-4-fluoro-5-oxopyrrolidin-2-yl)methoxy)-7-methoxyisoquinoline-6-carboxamide, 316.000 mgs sorbitol, 240.000 mgs sodium chloride, 64.000 mgs hydroxyethylcellulose, 72.000 mgs copovidone, 4.000 mgs magnesium stearate, and 4.000 mgs sodium stearyl fumarate resulting in a total active core weight of 800.000 mgs. The coating applied to the active core comprises 44.100 mgs cellulose acetate and 18.900 mgs polyethylene glycol resulting in a total coating weight of 63.000 mgs and a total tablet weight of 863.000 mgs. The 100 mg MR-FORM2 tablet may comprise 1-(((2S,3S,4S)-3-ethyl-4-fluoro-5-oxopyrrolidin-2-yl)methoxy)-7-methoxyisoquinoline-6-carboxamide in any pharmaceutically acceptable crystalline or amorphous form including hydrates, solvates, co-crystals, salts and combinations thereof wherein the total amount of 1-(((2S,3S,4S)-3-ethyl-4-fluoro-5-oxopyrrolidin-2-yl)methoxy)-7-methoxyisoquinoline-6-carboxamide is 100 mgs. It is to be understood that the coating covers nearly 100% of the active core except for the laser-drilled delivery port in the tablet that allows 1-(((2S,3S,4S)-3-ethyl-4-fluoro-5-oxopyrrolidin-2-yl)methoxy)-7-methoxyisoquinoline-6-carboxamide to exit the tablet under the appropriate physiological conditions.

The term “200 mg MR-FORM2,” as used herein, means an ECS single layer modified release tablet comprising an active core and a coating applied to the active core, wherein the active core comprises 200.000 mgs 1-(((2S,3S,4S)-3-ethyl-4-fluoro-5-oxopyrrolidin-2-yl)methoxy)-7-methoxyisoquinoline-6-carboxamide, 216.000 mgs sorbitol, 240.000 mgs sodium chloride, 64.000 hydroxyethylcellulose, 72.000 mgs copovidone, 4.000 mgs magnesium stearate, and 4.000 mgs sodium stearyl fumarate resulting in a total active core weight of 800.000 mgs. The coating applied to the active core comprises 44.100 mgs cellulose acetate and 18.900 mgs polyethylene glycol resulting in a total coating weight of 63.000 mgs and a total tablet weight of 863.000 mgs. The 200 mg MR-FORM2 tablet may comprise 1-(((2S,3S,4S)-3-ethyl-4-fluoro-5-oxopyrrolidin-2-yl)methoxy)-7-methoxyisoquinoline-6-carboxamide in any pharmaceutically acceptable crystalline or amorphous form including hydrates, solvates, co-crystals, salts and combinations thereof wherein the total amount of 1-(((2S,3S,4S)-3-ethyl-4-fluoro-5-oxopyrrolidin-2-yl)methoxy)-7-methoxyisoquinoline-6-carboxamide is 200.000 mgs. It is to be understood that the coating covers nearly 100% of the active core except for the laser-drilled delivery port in the tablet that allows 1-(((2S,3S,4S)-3-ethyl-4-fluoro-5-oxopyrrolidin-2-yl)methoxy)-7-methoxyisoquinoline-6-carboxamide to exit the tablet under the appropriate physiological conditions.

The term “100 mg MR-FORM3,” as used herein, means an ECS single layer modified release tablet comprising an active core and a coating applied to the active core, wherein the active core comprises 100.000 mgs of 1-(((2S,3S,4S)-3-ethyl-4-fluoro-5-oxopyrrolidin-2-yl)methoxy)-7-methoxyisoquinoline-6-carboxamide and further comprising 392.480 mgs dextrates, 243.270 mgs sodium chloride, 72.00 mgs hydroxyethylcellulose, 2.250 mgs colloidal silicon dioxide, 81.00 mgs copovidone, 4.50 mgs magnesium stearate, and 4.50 mgs sodium stearyl fumarate resulting in a total active core weight of 900.00 mgs. The coating applied to the active core comprises 28.860 mgs cellulose acetate and 8.140 mgs polyethylene glycol. The 100 mg MR-FORM3 tablet may comprise 1-(((2S,3S,4S)-3-ethyl-4-fluoro-5-oxopyrrolidin-2-yl)methoxy)-7-methoxyisoquinoline-6-carboxamide in any pharmaceutically acceptable crystalline or amorphous form including hydrates, solvates, co-crystals, salts and combinations thereof wherein the total amount of 1-(((2S,3S,4S)-3-ethyl-4-fluoro-5-oxopyrrolidin-2-yl)methoxy)-7-methoxyisoquinoline-6-carboxamide is 100 mgs. It is to be understood that the coating covers nearly 100% of the active core except for the laser-drilled delivery port in the tablet that allows 1-(((2S,3S,4S)-3-ethyl-4-fluoro-5-oxopyrrolidin-2-yl)methoxy)-7-methoxyisoquinoline-6-carboxamide to exit the tablet under the appropriate physiological conditions.

The term “200 mg MR-FORM3,” as used herein, means an ECS single layer modified release tablet comprising an active core and a coating applied to the active core, wherein the active core comprises 200.000 mgs 1-(((2S,3S,4S)-3-ethyl-4-fluoro-5-oxopyrrolidin-2-yl)methoxy)-7-methoxyisoquinoline-6-carboxamide, 330.750 mgs dextrates, 205.000 mgs sodium chloride, 72.000 mgs hydroxyethylcellulose, 2.250 mgs colloidal silicon dioxide, 81.000 mgs copovidone, 4.500 mgs magnesium stearate, and 4.500 mgs sodium stearyl fumarate resulting in a total active core weight of 900.000 mgs. The coating applied to the active core comprises 28.860 mgs cellulose acetate and 8.140 mgs polyethylene glycol resulting in a total coating weight of 37.000 mgs and a total tablet weight of 937.000 mgs. The 200 mg MR-FORM3 tablet may comprise 1-(((2S,3S,4S)-3-ethyl-4-fluoro-5-oxopyrrolidin-2-yl)methoxy)-7-methoxyisoquinoline-6-carboxamide in any pharmaceutically acceptable crystalline or amorphous form including hydrates, solvates, co-crystals, salts and combinations thereof wherein the total amount of 1-(((2S,3S,4S)-3-ethyl-4-fluoro-5-oxopyrrolidin-2-yl)methoxy)-7-methoxyisoquinoline-6-carboxamide is 200.000 mgs. It is to be understood that the coating covers nearly 100% of the active core except for the laser-drilled delivery port in the tablet that allows 1-(((2S,3S,4S)-3-ethyl-4-fluoro-5-oxopyrrolidin-2-yl)methoxy)-7-methoxyisoquinoline-6-carboxamide to exit the tablet under the appropriate physiological conditions.

The term “11 mg tablet of tofacitinib or 11 mg extended release tablet of tofacitinib,” as used herein means the formulations as described in U.S. Pat. No. 9,937,181 and includes the equivalent amount of tofacitinib as the citrate salt.

The term “at the inflammation site,” as used herein, means one or more inflammation sites.

The term “patient” or “subject,” as used herein, means a human being in need of the treatments or therapies as described herein.

The terms “PF-06650833-00, PF-06650833, '833, and 833” as used herein, mean 1-(((2S,3S,4S)-3-Ethyl-4-fluoro-5-oxopyrrolidin-2-yl)methoxy)-7-methoxyisoquinoline-6-carboxamide having the structure

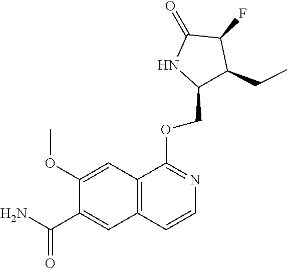 [SMILES: CC[C@@H]1[C@H](F)C(=O)N[C@@H]1COc1nccc2cc(C(N)=O)c(OC)cc12]

and includes any pharmaceutically acceptable crystalline or amorphous form including hydrates, solvates, co-crystals, salts and combinations thereof. Certain forms of 1-(((2S,3S,4S)-3-ethyl-4-fluoro-5-oxopyrrolidin-2-yl)methoxy)-7-methoxyisoquinoline-6-carboxamide may be prepared following the experimental procedures disclosed in WO2015/150995, Org. Process Res. Dev. 2018, 22, 1835-1845, and IP.COM disclosure no.: IPCOM000256080D (2018 Nov. 2), all three documents are herein incorporated by reference in their entirety.

The terms “PF-06651600-00, PF-06651600, '600 and 600,” as used herein, mean 1-((2S,5R)-5-((7H-Pyrrolo[2,3-d]pyrimidin-4-yl)amino)-2-methylpiperidin-1-yl)prop-2-en-1-one having structure

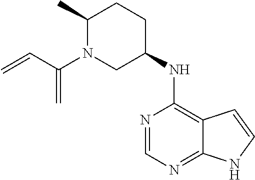 [SMILES: C=CC(=C)N1C[C@H](Nc2ncnc3[nH]ccc23)CC[C@@H]1C]

and includes any pharmaceutically acceptable crystalline or amorphous form including hydrates, solvates, co-crystals, salts and combinations thereof. Certain forms of 1-((2S,5R)-5-((7H-pyrrolo[2,3-d]pyrimidin-4-yl)amino)-2-methylpiperidin-1-yl)prop-2-en-1-one may be prepared following the experimental procedures disclosed in WO2015/083028 and Thorarensen et al., J. Med. Chem. 2017, 60, 1971-1993, both documents are herein incorporated by reference in their entirety.

The terms “PF-04524477-00, PF-04524477, 477, '477, tofacitinib, or tofa,” as used herein, mean 3-((3R,4R)-4-Methyl-3-(methyl(7H-pyrrolo[2,3-d]pyrimidin-4-yl)amino)piperidin-1-yl)-3-oxopropanenitrile having structure

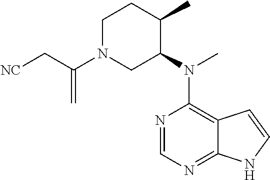 [SMILES: C=C(CC#N)N1CC[C@@H](C)[C@@H](N(C)c2ncnc3[nH]ccc23)C1]

and includes any pharmaceutically acceptable crystalline or amorphous form including hydrates, solvates, co-crystals, salts and combinations thereof. A preferred salt is the citrate salt approved in the U.S. under the brand XELJANZ™ and XELJANZ XR™. Certain forms of tofacitinib may be prepared following the experimental procedures disclosed in WO01/042246, WO02/096909, and WO03/048162, all three documents are herein incorporated by reference in their entirety.

The term “single layer,” as used herein, means the active core of the MR-FORM3 tablets comprising 1-(((2S,3S,4S)-3-Ethyl-4-fluoro-5-oxopyrrolidin-2-yl)methoxy)-7-methoxyisoquinoline-6-carboxamide, or a pharmaceutically acceptable salt thereof, and excipients blended together into a homogenous mixture, wherein the preferred excipients comprise one or more osmogens, a suspending agent, a glidant, a tableting aid, and one or more lubricants. Dextrates and sodium chloride are the preferred osmogens, hydroxyethylcellulose is the preferred suspending agent, colloidal silicon dioxide is the preferred glidant, copovidone is the preferred tableting agent, and magnesium stearate and sodium stearyl fumarate are the preferred lubricants.

The term “a coating applied to the active core,” as used herein, means the active core of the MR-FORM3 tablets are encompassed or surrounded by a coating, except for the delivery port, wherein the coating comprises an osmotic membrane and a plasticizer. Cellulose acetate is the preferred osmotic membrane and polyethylene glycol is the preferred plasticizer. The coating may be applied to the active core by means known in the art.

The term “delivery port,” as used herein, means a laser drilled hole through the coating that allows delivery of the active core outside of the tablet.

In another embodiment, 1-(((2S,3S,4S)-3-ethyl-4-fluoro-5-oxopyrrolidin-2-yl)methoxy)-7-methoxyisoquinoline-6-carboxamide is incorporated into an erodible or non-erodible polymeric matrix tablet. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous use environment, the erodible polymeric matrix imbibes water and forms an aqueous-swollen gel or “matrix” that entraps the 1-(((2S,3S,4S)-3-ethyl-4-fluoro-5-oxopyrrolidin-2-yl)methoxy)-7-methoxyisoquinoline-6-carboxamide. The aqueous-swollen matrix gradually erodes, swells, disintegrates, disperses or dissolves in the environment of use, thereby controlling the release of 1-(((2S,3S,4S)-3-ethyl-4-fluoro-5-oxopyrrolidin-2-yl)methoxy)-7-methoxyisoquinoline-6-carboxamide to the environment of use. Examples of such dosage forms are well known in the art. See, for example, Remington: The Science and Practice of Pharmacy, 20^{th} Edition, 2000.

A key ingredient of the water-swollen matrix is the water-swellable, erodible, or soluble polymer, which may generally be described as an osmopolymer, hydrogel or water-swellable polymer. Such polymers may be linear, branched, or crosslinked. They may be homopolymers or copolymers. Exemplary polymers include naturally occurring polysaccharides such as chitin, chitosan, dextran and pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; alginates such as ammonium alginate, sodium, potassium or calcium alginate, propylene glycol alginate; gelatin; collagen; and cellulosics. By “cellulosics” is meant a cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

Cellulosics for the erodible matrix comprise aqueous-soluble and aqueous-erodible cellulosics such as ethyl cellulose (EC), methylethyl cellulose (MEC), carboxymethyl cellulose (CMC), carboxymethyl ethylcellulose (CMEC), hydroxyethyl cellulose (HEC), hydroxypropyl cellulose (HPC), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC).

A particularly preferred class of such cellulosics comprises various grades of low viscosity (MW less than or equal to 50,000 daltons) and high viscosity (MW greater than 50,000 daltons) HPMC. Commercially available low viscosity HPMC polymers include the Dow METHOCEL™ series E3, E5, E15LV, E50LV and K100LV, while high viscosity HPMC polymers include E4MCR, E10MCR, K4M, K15M and K100M; especially preferred in this group are the METHOCEL™ K series. Other commercially available types of HPMC include the Shin Etsu METOLOSE™ 90SH series. In one embodiment, the HPMC has a low viscosity, meaning that the viscosity of a 2% (w/v) solution of the HPMC in water is less than about 120 cp. A preferred HPMC is one in which the viscosity of a 2% (w/v) solution of the HPMC in water ranges from 80 to 120 cp (such as METHOCEL™ K100LV).

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl) methacrylate chloride.

The erodible matrix polymer may also contain additives and excipients known in the pharmaceutical arts, including osmopolymers, osmogens, solubility-enhancing or -retarding agents and excipients that promote stability or processing of the dosage form.

In a non-erodible matrix system, 1-(((2S,3S,4S)-3-ethyl-4-fluoro-5-oxopyrrolidin-2-yl)methoxy)-7-methoxyisoquinoline-6-carboxamide is distributed in an inert matrix. The drug is released by diffusion through the inert matrix. Examples of materials suitable for the inert matrix include insoluble plastics, such as copolymers of ethylene and vinyl acetate, methyl acrylate-methyl methacrylate copolymers, polyvinyl chloride, and polyethylene; hydrophilic polymers, such as ethyl cellulose, cellulose acetate, and crosslinked polyvinylpyrrolidone (also known as crospovidone); and fatty compounds, such as carnauba wax, microcrystalline wax, and triglycerides. Such dosage forms are described further in Remington: The Science and Practice of Pharmacy, 20^{th} edition (2000).

In another embodiment, a matrix multiparticulate, comprises a plurality of 1-(((2S,3S,4S)-3-ethyl-4-fluoro-5-oxopyrrolidin-2-yl)methoxy)-7-methoxyisoquinoline-6-carboxamide-containing particles, each particle comprising a mixture of 1-(((2S,3S,4S)-3-ethyl-4-fluoro-5-oxopyrrolidin-2-yl)methoxy)-7-methoxyisoquinoline-6-carboxamide with one or more excipients selected to form a matrix capable of limiting the dissolution rate of the 1-(((2S,3S,4S)-3-ethyl-4-fluoro-5-oxopyrrolidin-2-yl)methoxy)-7-methoxyisoquinoline-6-carboxamide into an aqueous medium. The matrix materials useful for this embodiment are generally water-insoluble materials such as waxes, cellulose, or other water-insoluble polymers. If needed, the matrix materials may optionally be formulated with water-soluble materials which can be used as binders or as permeability-modifying agents. Matrix materials useful for the manufacture of these dosage forms include microcrystalline cellulose such as Avicel (registered trademark of FMC Corp., Philadelphia, Pa.), including grades of microcrystalline cellulose to which binders such as hydroxypropyl methyl cellulose have been added, waxes such as paraffin, modified vegetable oils, carnauba wax, hydrogenated castor oil, beeswax, and the like, as well as synthetic polymers such as poly(vinyl chloride), poly(vinyl acetate), copolymers of vinyl acetate and ethylene, polystyrene, and the like. Water soluble binders or release modifying agents which can optionally be formulated into the matrix include water-soluble polymers such as hydroxypropyl cellulose (HPC), hydroxypropyl methyl cellulose (HPMC), methyl cellulose, poly (N-vinyl-2-pyrrolidinone) (PVP), poly(ethylene oxide) (PEO), poly(vinyl alcohol) (PVA), xanthan gum, carrageenan, and other such natural and synthetic materials. In addition, materials which function as release-modifying agents include water-soluble materials such as sugars or salts. Preferred water-soluble materials include lactose, sucrose, glucose, and mannitol, as well as HPC, HPMC, and PVP.

A process for manufacturing matrix multiparticulates is the extrusion/spheronization process. For this process, the 1-(((2S,3S,4S)-3-ethyl-4-fluoro-5-oxopyrrolidin-2-yl)methoxy)-7-methoxyisoquinoline-6-carboxamide is wet-massed with a binder, extruded through a perforated plate or die, and placed on a rotating disk. The extrudate ideally breaks into pieces which are rounded into spheres, spheroids, or rounded rods on the rotating plate. Another process and composition for this method involves using water to wet-mass a blend.

Another process for manufacturing matrix multiparticulates is the preparation of wax granules. In this process, a desired amount of 1-(((2S,3S,4S)-3-ethyl-4-fluoro-5-oxopyrrolidin-2-yl)methoxy)-7-methoxyisoquinoline-6-carboxamide is stirred with liquid wax to form a homogeneous mixture, cooled and then forced through a screen to form granules. Preferred matrix materials are waxy substances. Some preferred waxy substances are hydrogenated castor oil and carnauba wax and stearyl alcohol.

A further process for manufacturing matrix multiparticulates involves using an organic solvent to aid mixing of the 1-(((2S,3S,4S)-3-ethyl-4-fluoro-5-oxopyrrolidin-2-yl)methoxy)-7-methoxyisoquinoline-6-carboxamide with the matrix material. This technique can be used when it is desired to utilize a matrix material with an unsuitably high melting point that, if the material were employed in a molten state, would cause decomposition of the drug or of the matrix material, or would result in an unacceptable melt viscosity, thereby preventing mixing of 1-(((2S,3S,4S)-3-ethyl-4-fluoro-5-oxopyrrolidin-2-yl)methoxy)-7-methoxyisoquinoline-6-carboxamide with the matrix material. 1-(((2S,3S,4S)-3-Ethyl-4-fluoro-5-oxopyrrolidin-2-yl)methoxy)-7-methoxyisoquinoline-6-carboxamide and matrix material may be combined with a modest amount of solvent to form a paste, and then forced through a screen to form granules from which the solvent is then removed. Alternatively, 1-(((2S,3S,4S)-3-ethyl-4-fluoro-5-oxopyrrolidin-2-yl)methoxy)-7-methoxyisoquinoline-6-carboxamide and matrix material may be combined with enough solvent to completely dissolve the matrix material and the resulting solution (which may contain solid drug particles) spray dried to form the particulate dosage form. This technique is preferred when the matrix material is a high molecular weight synthetic polymer such as a cellulose ether or cellulose ester. Solvents typically employed for the process include acetone, ethanol, isopropanol, ethyl acetate, and mixtures of two or more.

In another embodiment, the matrix multiparticulates are formed by the melt spray congeal process. The melt-congeal core comprises a matrix material. The matrix material serves two functions. First, the matrix material allows formation of relatively smooth, round cores that are amenable to coating. Second, the matrix material binds the optional excipients and/or drugs that may be incorporated into the core. The matrix material has the following physical properties: a sufficiently low viscosity in the molten state to form multiparticulates, as detailed below; and rapidly congeals to a solid when cooled below its melting point. For those multiparticulates incorporating drug in the core, the matrix preferably has a melting point below that of the melting point or decomposition point of the drug, and does not substantially dissolve the drug.

The melt-congeal cores consist essentially of a continuous phase of matrix material and optionally other excipients, with optional drug particles and optional swelling agent particles encapsulated within. Because of this, a sufficient amount of matrix material must be present to form smooth cores that are large enough to coat. In the case of cores containing solid particles, such as drug or swelling agent, the core must contain a sufficient amount of matrix material to encapsulate the drug and swelling agent to form relatively smooth and spherical cores, which are more easily coated by conventional spray-coating processes than irregularly-shaped ones.

In order to form small, smooth round cores, the matrix material must be capable of being melted and then atomized. The matrix material or mixture of materials is solid at 25 degrees C. However, the matrix material melts, or is capable of melting with the addition of an optional processing aid, at a temperature of less than 200 degrees centigrade so as to be suitable for melt-congeal processing described below. Preferably, the matrix material has a melting point between 50 degrees C. and 150° C. Although the term “melt” generally refers to the transition of a crystalline material from its crystalline to its liquid state, which occurs at its melting point, and the term “molten” generally refers to such a crystalline material in its fluid state, as used herein, the terms are used more broadly. In the case of “melt,” the term is used to refer to the heating of any material or mixture of materials sufficiently that it becomes fluid in the sense that it may be pumped or atomized in a manner similar to a crystalline material in the fluid state. Likewise “molten” refers to any material or mixture of materials that is in such a fluid state.

The matrix material is selected from the group consisting of waxes, long chain alcohols (Ci₂ or greater), fatty acid esters, glycolized fatty acid esters, phosphoglycerides, polyoxyethylene alkyl ethers, long chain carboxylic acids (Ci₂ or greater), sugar alcohols, and mixtures thereof. Exemplary matrix materials include highly purified forms of waxes, such as Carnauba wax, white and yellow beeswax, ceresin wax, microcrystalline wax, and paraffin wax; long-chain alcohols, such as stearyl alcohol, cetyl alcohol and polyethylene glycol; fatty acid esters (also known as fats or glycerides), such as isopropyl palmitate, isopropyl myristate, glyceryl monooleate, glyceryl monostearate, glyceryl palmitostearate, mixtures of mono-, di-, and trialkyl glycerides, including mixtures of glyceryl mono-, di-, and tribehenate, glyceryl tristearate, glyceryl tripalmitate and hydrogenated vegetable oils, including hydrogenated cottonseed oil; glycolized fatty acid esters, such as polyethylene glycol stearate and polyethylene glycol distearate; polyoxyethylene alkyl ethers; polyethoxylated castor oil derivatives; long-chain carboxylic acids such as stearic acid; and sugar alcohols such as mannitol and erythritol. The matrix material may comprise mixtures of materials, such as mixtures of any of the foregoing.

The core may also contain a variety of other excipients. One preferred excipient is a dissolution enhancer, which may be used to increase the rate of water uptake by the core and consequent expansion of the swelling agent. The dissolution enhancer is a different material than the matrix material. The dissolution enhancer may be in a separate phase or a single phase with the matrix material. Preferably, at least a portion of the dissolution enhancer is phase-separated from the matrix material. As water enters the core, the dissolution-enhancer dissolves, leaving channels which allow water to more rapidly enter the core. In general, dissolution enhancers are amphiphilic compounds and are generally more hydrophilic than the matrix materials. Examples of dissolution enhancers include: surfactants such as poloxamers, docusate salts, polyoxyethylene castor oil derivatives, polysorbates, sodium lauryl sulfate, and sorbitan monoesters; sugars, such as glucose, xylitol, sorbitol and maltitol; salts, such as sodium chloride, potassium chloride, lithium chloride, calcium chloride, magnesium chloride, sodium sulfate, potassium sulfate, sodium carbonate, magnesium sulfate and potassium phosphate; and amino acids, such as alanine and glycine; and mixtures thereof. One surfactant-type dissolution-enhancer is a poloxambetar (commercially available as the LUTROL or PLURONIC series from BASF Corp.).

The core may also contain other optional excipients, such as agents that inhibit or delay the release of drug from the multiparticulates. Such dissolution-inhibiting agents are generally hydrophobic and include dialkylphthalates such as dibutyl phthalate, and hydrocarbon waxes, such as microcrystalline wax and paraffin wax. Another useful class of excipients comprises materials that may be used to adjust the viscosity of the molten feed used to form the cores. Such viscosity-adjusting excipients will generally make up 0 to 25 wt percent of the core. The viscosity of the molten feed is a key variable in obtaining cores with a narrow particle size distribution. For example, when a spinning-disk atomizer is employed, it is preferred that the viscosity of the molten mixture be at least about 1 cp and less than about 10,000 cp, preferably at least 50 cp and less than about 1000 cp. If the molten mixture has a viscosity outside these ranges, a viscosity-adjusting agent can be added to obtain a molten mixture within the viscosity range. Examples of viscosity-reducing excipients include stearyl alcohol, cetyl alcohol, low molecular weight polyethylene glycol (i.e., less than about 1000 daltons), isopropyl alcohol, and water. Examples of viscosity-increasing excipients include microcrystalline wax, paraffin wax, synthetic wax, high molecular weight polyethylene glycols (i.e., greater than about 5000 daltons), ethyl cellulose, hydroxypropyl cellulose, hydroxypropyl methyl cellulose, methyl cellulose, silicon dioxide, microcrystalline cellulose, magnesium silicate, sugars, and salts.

For those embodiments containing a drug in the core, other excipients may be added to adjust the release characteristics of the drug from the cores. For example, an acid or base may be included in the composition to modify the rate at which drug is released in an aqueous use environment. Examples of acids or bases that can be included in the composition include citric acid, adipic acid, malic acid, fumaric acid, succinic acid, tartaric acid, di- and tribasic sodium phosphate, di- and tribasic calcium phosphate, mono-, di-, and triethanolamine, sodium bicarbonate and sodium citrate dihydrate.

Still other excipients may be added to improve processing, such as excipients to reduce the static charge on the cores or to reduce the melting temperature of the matrix material. Examples of such anti-static agents include talc and silicon dioxide. Flavorants, colorants, and other excipients may also be added in their usual amounts for their usual purposes.

The multiparticulates are made via a melt-congeal process comprising the steps: (a) forming a molten mixture comprising the drug, the glyceride (or other waxes), and any release modifying agents; (b) delivering the molten mixture of step (a) to an atomizing means to form droplets from the molten mixture; and (c) congealing the droplets from step (b) to form multiparticulates.

The processing conditions are chosen to maintain the crystallinity of the drug. The temperature of the molten mixture is kept below the melting point of the drug. Preferably, at least 70 wt percent of the drug remains crystalline within the molten feed, more preferably, at least 80 wt percent and most preferably at least 90 wt percent.

The term “molten mixture” as used herein refers to a mixture of drug, glyceride (or other waxes), and any release modifying agents required heated sufficiently that the mixture becomes sufficiently fluid that the mixture may be formed into droplets or atomized. Atomization of the molten mixture may be carried out using any of the atomization methods described below. Generally, the mixture is molten in the sense that it will flow when subjected to one or more forces such as pressure, shear, and centrifugal force, such as that exerted by a centrifugal or spinning-disk atomizer. Thus, the drug/glyceride/release-modifying agent mixture may be considered “molten” when any portion of the drug/glyceride/release-modifying agent mixture becomes sufficiently fluid that the mixture, as a whole, may be atomized. Generally, a mixture is sufficiently fluid for atomization when the viscosity of the molten mixture is less than about 20,000 cp. Often, the mixture becomes molten when the mixture is heated above the melting point of the glyceride/release-modifying agent mixture, in cases where the glyceride/release-modifying agent mixture is sufficiently crystalline to have a relatively sharp melting point; or, when the glyceride/release-modifying agent mixture is amorphous, above the softening point of the glyceride/release-modifying agent mixture. The molten mixture is therefore often a suspension of solid particles in a fluid matrix. In one preferred embodiment, the molten mixture comprises a mixture of substantially crystalline drug particles suspended in a glyceride/release-modifying agent mixture that is substantially fluid. In such cases, a portion of the drug may be dissolved in the glyceride/release-modifying agent mixture and a portion of the glyceride/release-modifying agent mixture may remain solid.

Virtually any process may be used to form the molten mixture. One method involves heating the glyceride/release-modifying agent mixture in a tank until it is fluid and then adding the drug to the molten glyceride/release-modifying agent mixture. Generally, the glyceride/release-modifying agent mixture is heated to a temperature of about 10 degrees C. or more above the temperature at which it becomes fluid. When one or more of the glyceride/release-modifying agent components is crystalline, this is generally about 10 degrees C. or more above the melting point of the lowest melting point material of the mixture. The process is carried out so that at least a portion of the feed remains fluid until atomized. Once the glyceride/release-modifying agent mixture has become fluid, the drug may be added to the fluid carrier or “melt.” Although the term “melt” generally refers specifically to the transition of a crystalline material from its crystalline to its liquid state, which occurs at its melting point, and the term “molten” generally refers to such a crystalline material in its fluid state, as used herein, the terms are used more broadly, referring in the case of “melt” to the heating of any material or mixture of materials sufficiently that it becomes fluid in the sense that it may be pumped or atomized in a manner similar to a crystalline material in the fluid state. Likewise “molten” refers to any material or mixture of materials that is in such a fluid state. Alternatively, the drug, the glyceride (or other wax), and the release-modifying agent may be added to the tank and the mixture heated until the mixture has become fluid.

Once the glyceride/release-modifying agent mixture has become fluid and the drug has been added, the molten mixture is mixed to ensure the drug is uniformly distributed therein. Mixing is generally done using mechanical means, such as overhead mixers, magnetically driven mixers and stir bars, planetary mixers, and homogenizers. Optionally, the contents of the tank can be pumped out of the tank and through an in-line, static mixer or extruder and then returned to the tank. The amount of shear used to mix the molten feed should be sufficiently high to ensure uniform distribution of the drug in the molten carrier. The amount of shear is kept low enough so the form of the drug does not change, i.e., so as to cause an increase in the amount of amorphous drug or a change in the crystalline form of the drug. It is also preferred that the shear not be so high as to reduce the particle size of the drug crystals. The molten mixture can be mixed from a few minutes to several hours, the mixing time being dependent on the viscosity of the feed and the solubility of drug and any optional excipients in the carrier.

An alternative method of preparing the molten mixture is to use two tanks, melting either the glyceride (or other waxes) or the release-modifying agent in one tank and the other component in another tank. The drug is added to one of these tanks and mixed as described above. The two melts are then pumped through an in-line static mixer or extruder to produce a single molten mixture that is directed to the atomization process described below.

Another method that can be used to prepare the molten mixture is to use a continuously stirred tank system. In this system, the drug, glyceride (or other waxes), and release-modifying agent are continuously added to a heated tank equipped with means for continuous stirring, while the molten feed is continuously removed from the tank. The contents of the tank are heated such that the temperature of the contents is about 10 degrees C. or more above the melting point of the carrier. The drug, glyceride (or other waxes), and release-modifying agent are added in such proportions that the molten mixture removed from the tank has the desired composition. The drug is typically added in solid form and may be pre-heated prior to addition to the tank. The glyceride (or other waxes), and release-modifying agent may also be preheated or even pre-melted prior to addition to the continuously stirred tank system.

In another method for forming the molten mixture is by an extruder. By “extruder” is meant a device or collection of devices that creates a molten extrudate by heat and/or shear forces and/or produces a uniformly mixed extrudate from a solid and/or liquid (e.g., molten) feed. Such devices include, but are not limited to single-screw extruders; twin-screw extruders, including co-rotating, counter-rotating, intermeshing, and non-intermeshing extruders; multiple screw extruders; ram extruders, consisting of a heated cylinder and a piston for extruding the molten feed; gear-pump extruders, consisting of a heated gear pump, generally counter-rotating, that simultaneously heats and pumps the molten feed; and conveyer extruders.

Conveyer extruders comprise a conveyer means for transporting solid and/or powdered feeds, such, such as a screw conveyer or pneumatic conveyer, and a pump. At least a portion of the conveyer means is heated to a sufficiently high temperature to produce the molten mixture. The molten mixture may optionally be directed to an accumulation tank, before being directed to a pump, which directs the molten mixture to an atomizer. Optionally, an in-line mixer may be used before or after the pump to ensure the molten mixture is substantially homogeneous. In each of these extruders the molten mixture is mixed to form a uniformly mixed extrudate. Such mixing may be accomplished by various mechanical and processing means, including mixing elements, kneading elements, and shear mixing by backflow. Thus, in such devices, the composition is fed to the extruder, which produces a molten mixture that can be directed to the atomizer.

In another embodiment, the composition is fed to the extruder in the form of a solid powder. The powdered feed can be prepared using methods well known in the art for obtaining powdered mixtures with high content uniformity. Generally, it is desirable that the particle sizes of the drug, glyceride (or other waxes), and release-modifying agent be similar to obtain a substantially uniform blend. However, this is not essential to the successful practice of the invention.

An example of a process for preparing a substantially uniform blend is as follows. First, the glyceride (or other waxes) and release-modifying agent are milled so that their particle sizes are about the same as that of the drug; next, the drug, glyceride (or other waxes), and release-modifying agent are blended in a V-blender for 20 minutes; the resulting blend is then de-lumped to remove large particles; the resulting blend is finally blended for an additional 4 minutes. In some cases it is difficult to mill the glyceride (or other waxes), and release-modifying agent to the desired particle size since many of these materials tend to be waxy substances and the heat generated during the milling process can gum up the milling equipment. In such cases, small particles of the glyceride (or other waxes), and release-modifying agent can be formed using a melt- or spray-congeal process, as described below. The resulting congealed particles of glyceride (or other waxes), and release-modifying agent can then be blended with the drug to produce the feed for the extruder.

Another method for producing the feed to the extruder is to melt the glyceride (or other waxes) and release-modifying agent in a tank, mix in the drug as described above for the tank system, and then cool the molten mixture, producing a solidified mixture of drug and carrier. This solidified mixture can then be milled to a uniform particle size and fed to the extruder.

A two-feed extruder system can also be used to produce the molten mixture. In this system the drug, glyceride (or other waxes) and release-modifying agent, all in powdered form, are fed to the extruder through the same or different feed ports. In this way, the need for blending the components is eliminated.

Alternatively, the glyceride (or other waxes) and release-modifying agent in powder form may be fed to the extruder at one point, allowing the extruder to melt the glyceride (or other waxes) and release-modifying agent. The drug is then added to the molten glyceride (or other waxes) and release-modifying agent through a second feed delivery port part way along the length of the extruder, thus minimizing the contact time of the drug with the molten glyceride (or other waxes) and release-modifying agent. The closer the second feed delivery port is to the extruder exit, the lower is the residence time of drug in the extruder. Multiple-feed extruders can be used when optional excipients are included in the multiparticulate.

In another method, the composition is in the form of large solid particles or a solid mass, rather than a powder, when fed to the extruder. For example, a solidified mixture can be prepared as described above and then molded to fit into the cylinder of a ram extruder and used directly without milling.

In another method, the glyceride (or other waxes) and release-modifying agent can be first melted in, for example, a tank, and fed to the extruder in molten form. The drug, typically in powdered form, may then be introduced to the extruder through the same or a different delivery port used to feed the glyceride (or other waxes) and release-modifying agent into the extruder. This system has the advantage of separating the melting step for the glyceride (or other waxes) and release-modifying agent from the mixing step, minimizing contact of the drug with the molten glyceride (or other waxes) and release-modifying agent.

In each of the above methods, the extruder should be designed such that it produces a molten mixture with the drug crystals uniformly distributed in the glyceride/release-modifying agent mixture. Generally, the temperature of the extrudate should be about 10 degrees C. or more above the temperature at which the drug and carrier mixture becomes fluid. The various zones in the extruder should be heated to appropriate temperatures to obtain the desired extrudate temperature as well as the desired degree of mixing or shear, using procedures well known in the art. As discussed above for mechanical mixing, a minimum shear should be used to produce a uniform molten mixture, such that the crystalline form of the drug is unchanged and that dissolution or formation of amorphous drug is minimized.

The feed is preferably molten prior to congealing for at least 5 seconds, more preferably at least 10 seconds, and most preferably at least 15 seconds, so as to ensure adequate homogeneity of the drug/glyceride/release-modifying agent melt. It is also preferred that the molten mixture remain molten for no more than about 20 minutes to limit exposure of the drug to the molten mixture. As described above, depending on the reactivity of the chosen glyceride/release-modifying agent mixture, it may be preferable to further reduce the time that the mixture is molten to well below 20 minutes in order to limit drug degradation to an acceptable level. In such cases, such mixtures may be maintained in the molten state for less than 15 minutes, and in some cases, even less than 10 minutes. When an extruder is used to produce the molten feed, the times above refer to the mean time from when material is introduced to the extruder to when the molten mixture is congealed. Such mean times can be determined by procedures well known in the art. In one exemplary method, a small amount of dye or other similar compound is added to the feed while the extruder is operating under nominal conditions. Congealed multiparticulates are then collected overtime and analyzed for the dye, from which the mean time is determined.

Once the molten mixture has been formed, it is delivered to an atomizer that breaks the molten feed into small droplets. Virtually any method can be used to deliver the molten mixture to the atomizer, including the use of pumps and various types of pneumatic devices (e.g., pressurized vessels, piston pots). When an extruder is used to form the molten mixture, the extruder itself can be used to deliver the molten mixture to the atomizer. Typically, the molten mixture is maintained at an elevated temperature while delivering the mixture to the atomizer to prevent solidification of the mixture and to keep the molten mixture flowing.

Generally, atomization occurs in one of several ways, including (1) by “pressure” or single-fluid nozzles; (2) by two-fluid nozzles; (3) by centrifugal or spinning-disk atomizers, (4) by ultrasonic nozzles; and (5) by mechanical vibrating nozzles. Detailed descriptions of atomization processes can be found in Lefebvre, Atomization and Sprays (1989) or in Perry's Chemical Engineers' Handbook (7th Ed. 1997). Preferably, a centrifugal or spinning-disk atomizer is used, such as the FX1 100-mm rotary atomizer manufactured by Niro A/S (Soeborg, Denmark).

Once the molten mixture has been atomized, the droplets are congealed, typically by contact with a gas or liquid at a temperature below the solidification temperature of the droplets. Typically, it is desirable that the droplets are congealed in less than about 60 seconds, preferably in less than about 10 seconds, more preferably in less than about 1 second. Often, congealing at ambient temperature results in sufficiently rapid solidification of the droplets. However, the congealing step often occurs in an enclosed space to simplify collection of the multiparticulates. In such cases, the temperature of the congealing media (either gas or liquid) will increase over time as the droplets are introduced into the enclosed space, potentially effecting the formation of the multiparticulates or the chemical stability of the drug. Thus, a cooling gas or liquid is often circulated through the enclosed space to maintain a constant congealing temperature. When it is desirable to minimize the time the drug is exposed to high temperatures, e.g., to prevent degradation, the cooling gas or liquid can be cooled to below ambient temperature to promote rapid congealing, thus minimizing formation of degradants.

Following formation of the multiparticulates, it may be desired to post-treat the multiparticulates to improve drug crystallinity and/or the stability of the multiparticulate.

The multiparticulates may also be mixed or blended with one or more pharmaceutically acceptable materials to form a suitable dosage form. Suitable dosage forms include tablets, capsules, sachets, oral powders for constitution, and the like.

Following formation of the melt spray congeal multiparticulates, the multiparticulates may optionally be coated with an additional exterior coating. The exterior coating may be any conventional coating, such as a protective film coating, a coating to provide delayed or sustained release of the drug, or to provide tastemasking.

In another embodiment, the coating is an enteric coating to provide delayed release of the drug. By “enteric coating” is meant an acid resistant coating that remains intact and does not dissolve at pH of less than about 4. The enteric coating surrounds the multiparticulate so that the solid amorphous dispersion layer does not dissolve or erode in the stomach. The enteric coating may include an enteric coating polymer. Enteric coating polymers are generally polyacids having a pKₐ of about 3 to 5. Examples of enteric coating polymers include: cellulose derivatives, such as cellulose acetate phthalate, cellulose acetate trimellitate, hydroxypropyl methyl cellulose acetate succinate, cellulose acetate succinate, carboxy methyl ethyl cellulose, methylcellulose phthalate, and ethylhydroxy cellulose phthalate; vinyl polymers, such as polyvinyl acetate phthalate, vinyl acetate-maleic anhydride copolymer; polyacrylates; and polymethacrylates such as methyl acrylate-methacrylic acid copolymer, methacrylate-methacrylic acid-octyl acrylate copolymer; and styrene-maleic mono-ester copolymer. These may be used either alone or in combination, or together with other polymers than those mentioned above.

One class of enteric coating materials are the pharmaceutically acceptable methacrylic acid copolymer which are copolymers, anionic in character, based on methacrylic acid and methyl methacrylate. Some of these polymers are known and sold as enteric polymers, for example having a solubility in aqueous media at pH 5.5 and above, such as the commercially available EUDRAGIT enteric polymers, such as Eudragit L 30, a polymer synthesized from dimethylaminoethyl methacrylate and Eudragit S and Eudragit FS.

The exterior coatings may include conventional plasticizers, including dibutyl phthalate; dibutyl sebacate; diethyl phthalate; dimethyl phthalate; triethyl citrate; benzyl benzoate; butyl and glycol esters of fatty acids; mineral oil; oleic acid; stearic acid; cetyl alcohol; stearyl alcohol; castor oil; corn oil; coconut oil; and camphor oil; and other excipients such as anti-tack agents, glidants, etc. For plasticizers, triethyl citrate, coconut oil and dibutyl sebacate are particularly preferred.

Exterior coatings can be formed using solvent-based and hot-melt coating processes. In solvent-based processes, the coating is made by first forming a solution or suspension comprising the solvent, the coating material and optional coating additives. The coating materials may be completely dissolved in the coating solvent, or only dispersed in the solvent as an emulsion or suspension or a combination of the two. Latex dispersions are an example of an emulsion or suspension that may be useful as in a solvent-based coating process. In one aspect, the solvent is a liquid at room temperature.

Coating may be conducted by conventional techniques, such as by pan coaters, rotary granulators and fluidized bed coaters such as top-spray, tangential-spray or bottom-spray (Wurster coating). A top-spray method can also be used to apply the coating. In this method, coating solution is sprayed down onto the fluidized cores. The solvent evaporates from the coated cores and the coated cores are re-fluidized in the apparatus. Coating continues until the desired coating thickness is achieved. Compositions and methods for making the multiparticulates of this embodiment are detailed in the following US patent applications, US 2005-0181062, US 2005-0181062, US 2008-0199527, US 2005-0186285A1 which are herein incorporated as reference in their entirety.

The multiparticulates of the present invention are particularly suitable for controlled release or delayed release or any combination of these two release profiles when introduced to a use environment. As used herein, a “use environment” can be either the in vivo environment of the gastrointestinal (GI) tract or the in vitro dissolution tests described herein. Information about in vivo release rates can be determined from the pharmacokinetic profile using standard deconvolution or Wagner-Nelson treatment of the data which should be readily known to those skilled in the art.

Once the 1-(((2S,3S,4S)-3-ethyl-4-fluoro-5-oxopyrrolidin-2-yl)methoxy)-7-methoxyisoquinoline-6-carboxamide matrix multiparticulates are formed through methods described above, they may be blended with compressible excipients such as lactose, microcrystalline cellulose, dicalcium phosphate, and the like and the blend compressed to form a tablet or capsule. Disintegrants such as sodium starch glycolate or crosslinked poly(vinyl pyrrolidone) are also usefully employed. Tablets or capsules prepared by this method disintegrate when placed in an aqueous medium (such as the GI tract), thereby exposing the multiparticulate matrix which releases 1-(((2S,3S,4S)-3-ethyl-4-fluoro-5-oxopyrrolidin-2-yl)methoxy)-7-methoxyisoquinoline-6-carboxamide there from.

Other conventional formulation excipients may be employed in the controlled release portion of the invention, including those excipients well known in the art, e.g., as described in Remington: The Science and Practice of Pharmacy, 20^{th} edition (2000). Generally, excipients such as surfactants, pH modifiers, fillers, matrix materials, complexing agents, solubilizers, pigments, lubricants, glidants, flavorants, and so forth may be used for customary purposes and in typical amounts without adversely affecting the properties of the compositions.

Example matrix materials, fillers, or diluents include lactose, mannitol, xylitol, dextrose, sucrose, sorbitol, compressible sugar, microcrystalline cellulose, powdered cellulose, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, polyethylene oxide, hydroxypropyl methyl cellulose and mixtures thereof.

In another embodiment, 1-(((2S,3S,4S)-3-ethyl-4-fluoro-5-oxopyrrolidin-2-yl)methoxy)-7-methoxyisoquinoline-6-carboxamide is incorporated into osmotic delivery devices or “osmotic pumps” as they are known in the art. Osmotic pumps comprise a core containing an osmotically effective composition surrounded by a semipermeable membrane. The term “semipermeable” in this context means that water can readily diffuse through the membrane, but solutes dissolved in water typically cannot readily diffuse through the membrane relative to the rate of water diffusion through the membrane. In use, when placed in an aqueous environment, the device imbibes water due to the osmotic activity of the core composition. Owing to the semipermeable nature of the surrounding membrane, the contents of the device (including 1-(((2S,3S,4S)-3-ethyl-4-fluoro-5-oxopyrrolidin-2-yl)methoxy)-7-methoxyisoquinoline-6-carboxamide and any excipients) cannot pass through the non-porous regions of the membrane and are driven by osmotic pressure to leave the device through an opening or passageway pre-manufactured into the dosage form or, alternatively, formed in situ in the GI tract as by the bursting of intentionally-incorporated weak points in the coating under the influence of osmotic pressure. The osmotically effective composition includes water-soluble species, which generate a colloidal osmotic pressure, and water-swellable polymers. Examples of such dosage forms are well known in the art. See, for example, Remington: The Science and Practice of Pharmacy, 21^{st} Edition, 2006 Chapter 47; page 950-1 and herein incorporated as reference.

In one embodiment of the present invention, 1-(((2S,3S,4S)-3-ethyl-4-fluoro-5-oxopyrrolidin-2-yl)methoxy)-7-methoxyisoquinoline-6-carboxamide is incorporated into a bilayer osmotic delivery device such that the 1-(((2S,3S,4S)-3-ethyl-4-fluoro-5-oxopyrrolidin-2-yl)methoxy)-7-methoxyisoquinoline-6-carboxamide-containing composition must include an entraining agent in the form of a water-swellable polymer and a second push layer or water swelling layer which contains water-swellable polymers and/or osmoticallly active agents, but does not contain any active agent. The bilayer tablet or capsule is surrounded by a semi-permeable membrane which contains one or more openings which are manufactured into the dosage form through such techniques as laser drilling. Such water-swellable polymers are often referred to in the pharmaceutical arts as an “osmopolymer” or a “hydrogel.” The entraining agent suspends or entrains the drug so as to aid in the delivery of the drug through the delivery port(s). While not wishing to be bound by any particular theory, it is believed that upon the imbibition of water into the dosage form, the entraining agent has enough viscosity to allow it to suspend or entrain the drug, while at the same time remaining sufficiently fluid to allow the entraining agent to pass through the delivery port(s) along with the drug. The entraining agent may be a single material or a mixture of materials. Non-crosslinked polyethylene oxide (PEO) may be used as the entraining agent. Other suitable entraining agents include hydroxypropyl cellulose (HPC), hydroxypropylmethyl cellulose (HPMC), methylcellulose (MC), hydroxyethyl cellulose (HEC) and polyvinyl pyrrolidone (PVP), as well as mixtures of these polymers with PEO.

The choice of the molecular weight for the PEO depends in part on whether the PEO makes up the bulk of the non-1-(((2S,3S,4S)-3-ethyl-4-fluoro-5-oxopyrrolidin-2-yl)methoxy)-7-methoxyisoquinoline-6-carboxamide portion of the 1-(((2S,3S,4S)-3-ethyl-4-fluoro-5-oxopyrrolidin-2-yl)methoxy)-7-methoxyisoquinoline-6-carboxamide-containing composition, or whether significant amounts of other low-molecular weight water-soluble excipients are included; that is, the PEO molecular weight choice depends on the fraction of the 1-(((2S,3S,4S)-3-ethyl-4-fluoro-5-oxopyrrolidin-2-yl)methoxy)-7-methoxyisoquinoline-6-carboxamide-containing composition that is PEO. Should the 1-(((2S,3S,4S)-3-ethyl-4-fluoro-5-oxopyrrolidin-2-yl)methoxy)-7-methoxyisoquinoline-6-carboxamide-containing composition not become fluid rapidly, the dosage form can swell and rupture the coating that surrounds the core, potentially causing failure of the dosage form. Where the excipients of the 1-(((2S,3S,4S)-3-ethyl-4-fluoro-5-oxopyrrolidin-2-yl)methoxy)-7-methoxyisoquinoline-6-carboxamide-containing composition are primarily PEO. Preferred fluidizing agents are low molecular weight, water-soluble solutes such as non-reducing sugars and organic acids with aqueous solubilities of 30 mg/mL or greater. Suitable sugars include xylitol, mannitol, sorbitol, and maltitol. Salts useful as a fluidizing agent include sodium chloride, sodium lactate and sodium acetate. Organic acids useful as a fluidizing agent include adipic acid, citric acid, malic acid, fumaric acid, succinic acid and tartaric acid.

The 1-(((2S,3S,4S)-3-ethyl-4-fluoro-5-oxopyrrolidin-2-yl)methoxy)-7-methoxyisoquinoline-6-carboxamide-containing composition may also contain other water-swellable polymers. For example, the 1-(((2S,3S,4S)-3-ethyl-4-fluoro-5-oxopyrrolidin-2-yl)methoxy)-7-methoxyisoquinoline-6-carboxamide-containing composition may contain relatively small amounts of water-swellable polymers that greatly expand in the presence of water. Such water-swellable polymers include sodium starch glycolate, sold under the trade name EXPLOTAB, and crosscarmellose sodium, sold under the trade name AC-DI-SOL.

The 1-(((2S,3S,4S)-3-ethyl-4-fluoro-5-oxopyrrolidin-2-yl)methoxy)-7-methoxyisoquinoline-6-carboxamide-containing composition may optionally include osmotically effective solutes, often referred to as “osmogens” or “osmagents.” Typical classes of suitable osmogens are water-soluble salts, sugars, organic acids, and other low-molecule-weight organic compounds that are capable of imbibing water to thereby establish an osmotic pressure gradient across the barrier of the surrounding coating. Typical useful salts include magnesium sulfate, magnesium chloride, calcium chloride, sodium chloride, lithium chloride, potassium sulfate, sodium carbonate, sodium sulfite, lithium sulfate, potassium chloride, and sodium sulfate. Conventionally, chloride salts such as sodium chloride are utilized as osmogens.

The 1-(((2S,3S,4S)-3-ethyl-4-fluoro-5-oxopyrrolidin-2-yl)methoxy)-7-methoxyisoquinoline-6-carboxamide-containing composition may further include solubility-enhancing agents or solubilizers that promote the aqueous solubility of the drug. Solubilizers useful with 1-(((2S,3S,4S)-3-ethyl-4-fluoro-5-oxopyrrolidin-2-yl)methoxy)-7-methoxyisoquinoline-6-carboxamide include organic acids and organic acid salts, partial glycerides, e.g., less than fully esterified derivatives of glycerin, including glycerides, monoglycerides, diglycerides, glyceride derivatives, polyethylene glycol esters, polypropylene glycol esters, polyhydric alcohol esters, polyoxyethylene ethers, sorbitan esters, polyoxyethylene sorbitan esters, and carbonate salts.

A preferred class of solubilizers is organic acids. There are a variety of factors to consider when choosing an appropriate organic acid for use as a solubilizer with 1-(((2S,3S,4S)-3-ethyl-4-fluoro-5-oxopyrrolidin-2-yl)methoxy)-7-methoxyisoquinoline-6-carboxamide in an osmotic dosage form. The acid should not interact adversely with 1-(((2S,3S,4S)-3-ethyl-4-fluoro-5-oxopyrrolidin-2-yl)methoxy)-7-methoxyisoquinoline-6-carboxamide, should have appropriate water solubility, and should provide good manufacturing properties.

The water-swellable composition may also optionally contain a colorant. The purpose of the colorant is to allow identification of the drug-containing side of the tablet face for purposes of providing the delivery port, such as by laser drilling through the coating. Acceptable colorants include, but are not limited to, Red Lake No. 40, FD C Blue 2 and FD C Yellow 6.

The 1-(((2S,3S,4S)-3-ethyl-4-fluoro-5-oxopyrrolidin-2-yl)methoxy)-7-methoxyisoquinoline-6-carboxamide-containing layer and/or the water-swellable composition layer and/or the functional rate controlling membrane may optionally contain an antioxidant, such as but not limited to BHT, BHA, sodium metabisulfite, propyl galate, glycerin, vitamin E, Citric Acid or ascorbyl palmitate. For additional examples of antioxidants, see C.-M. Andersson, A. Hallberg, and T. Hoegberg. Advances in the development of pharmaceutical antioxidants. Advances in Drug Research. 28:65-180, 1996.

Water-swellable composition may also include other conventional pharmaceutically useful excipients such as a binder, including HPC, HPMC, HEC, MC, and PVP, a tableting aid, such as microcrystalline cellulose, and a lubricant such as magnesium stearate.

The water-swellable composition is prepared by mixing the water-swellable polymer and the other excipients to form a uniform blend. To obtain a uniform blend, it is desirable to either wet or dry granulate or dry blend ingredients that have similar particle sizes using the types of processes known to those skilled in the art.

The core is prepared by first placing a mixture of the 1-(((2S,3S,4S)-3-ethyl-4-fluoro-5-oxopyrrolidin-2-yl)methoxy)-7-methoxyisoquinoline-6-carboxamide-containing composition into a tablet press and then leveling the mixture by gentle compression. The water-swellable composition is then placed on top of the 1-(((2S,3S,4S)-3-ethyl-4-fluoro-5-oxopyrrolidin-2-yl)methoxy)-7-methoxyisoquinoline-6-carboxamide-containing composition and compressed in order to complete formation of the core. Alternatively, the water-swellable composition can be placed into the tablet press first, followed by the 1-(((2S,3S,4S)-3-ethyl-4-fluoro-5-oxopyrrolidin-2-yl)methoxy)-7-methoxyisoquinoline-6-carboxamide-containing composition.

The respective amounts of 1-(((2S,3S,4S)-3-ethyl-4-fluoro-5-oxopyrrolidin-2-yl)methoxy)-7-methoxyisoquinoline-6-carboxamide-containing composition and water-swellable composition are chosen to provide satisfactory 1-(((2S,3S,4S)-3-ethyl-4-fluoro-5-oxopyrrolidin-2-yl)methoxy)-7-methoxyisoquinoline-6-carboxamide release.

Following formation of the core, the semi-permeable coating is applied. The coating should have high water permeability and a high strength, while at the same time be easily fabricated and applied. High water permeability is required to permit water to enter the core in sufficient volume. High strength is required to ensure the coating does not burst when the core swells as it imbibes water, leading to an uncontrolled delivery of the core contents. Finally, the coating must have high reproducibility and yield.

It is essential that the coating have at least one delivery port in communication with the interior and exterior of the coating for delivery of the 1-(((2S,3S,4S)-3-ethyl-4-fluoro-5-oxopyrrolidin-2-yl)methoxy)-7-methoxyisoquinoline-6-carboxamide-containing composition.

Furthermore, the coating must be non-dissolving and non-eroding during release of the 1-(((2S,3S,4S)-3-ethyl-4-fluoro-5-oxopyrrolidin-2-yl)methoxy)-7-methoxyisoquinoline-6-carboxamide-containing composition, generally meaning that it be water-insoluble, such that 1-(((2S,3S,4S)-3-ethyl-4-fluoro-5-oxopyrrolidin-2-yl)methoxy)-7-methoxyisoquinoline-6-carboxamide is substantially entirely delivered through the delivery port(s), in contrast to delivery via permeation through the coating.

Coatings with these characteristics may be obtained using hydrophilic polymers such as plasticized and unplasticized cellulose esters, ethers, and ester-ethers. Particularly suitable polymers include cellulose acetate (CA), cellulose acetate butyrate (CAB), and ethyl cellulose (EC). One set of polymers are cellulose acetates having acetyl contents of 25 to 42%. One typical polymer is CA having an acetyl content of 39.8%, specifically, CA 398-10 (Eastman Fine Chemicals, Kingsport, Tenn.). CA 398-10 is reported to have an average molecular weight of about 40,000 daltons. Another typical CA having an acetyl content of 39.8% is high molecular weight CA having an average molecular weight greater than about 45,000, and specifically, CA 398-30 (Eastman Fine Chemical) which is reported to have an average molecular weight of 50,000 daltons.

Coating is conducted in conventional fashion by first forming a coating solution and then coating by dipping, fluidized bed coating, or by pan coating. To accomplish this, a coating solution is formed comprising the polymer and a solvent. Typical solvents useful with the cellulosic polymers above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, and mixtures thereof. The coating solution typically contains 2 to 15 wt % of the polymer.

The coating solution may also include pore-formers or non-solvents in any amount as long as the polymer remains soluble at the conditions used to form the coating and as long as the coating remains water permeable and has sufficient strength. Pore-formers and their use in fabricating coatings are described in U.S. Pat. Nos. 5,698,220 and 5,612,059, the pertinent disclosures of which are incorporated herein by reference. The term “pore former,” as used herein, refers to a material added to the coating solution that has low or no volatility relative to the solvent such that it remains as part of the coating following the coating process but that is sufficiently water swellable or water soluble such that, in the aqueous use environment it provides a water-filled or water-swollen channel or “pore” to allow the passage of water, thereby enhancing the water permeability of the coating. Suitable pore formers include but are not limited to hydroxypropylcellulose (HPC), polyethylene glycol (“PEG”), PVP, and PEO. To obtain a combination of high water permeability and high strength when PEG or HPC are used as a pore former, the weight ratio of CA:PEG or CA:HPC should range from about 6:4 to about 9:1.

The addition of a non-solvent such as water to the coating solution results in exceptional performance. By “non-solvent” is meant any material added to the coating solution that substantially dissolves in the coating solution and reduces the solubility of the coating polymer or polymers in the solvent. In general, the function of the non-solvent is to impart porosity to the resulting coating. As described below, porous coatings have higher water permeability than an equivalent weight of a coating of the same composition that is not porous and this porosity is indicated by a reduction in the density of the coating (mass/volume). Although not wishing to be bound by any particular mechanism of pore formation, it is generally believed that addition of a non-solvent imparts porosity to the coating during evaporation of solvent by causing the coating solution to undergo liquid and liquid phase separation prior to solidification. The suitability and amount of a particular candidate material can be evaluated for use as a non-solvent by progressively adding the candidate non-solvent to the coating solution until it becomes cloudy. If this does not occur at any addition level up to about 50 wt % of the coating solution, it generally is not appropriate for use as a non-solvent. When clouding is observed, termed the “cloud point,” an appropriate level of non-solvent for maximum porosity is the amount just below the cloud point. For acetone solutions comprising 7 wt % CA and 3 wt % PEG, the cloud point is at about 23 wt % water. When lower porosities are desired, the amount of non-solvent can be reduced as low as desired.

Suitable non-solvents are any materials that have appreciable solubility in the solvent and that lower the coating polymer solubility in the solvent. The preferred non-solvent depends on the solvent and the coating polymer chosen. In the case of using a volatile polar coating solvent such as acetone, suitable non-solvents include water, glycerol, alcohols such as methanol or ethanol.

When using CA 398-10, coating solution weight ratios of CA:PEG 3350:water are 2.4:1.6:5, 2.8:1.2:5, 3.2:0.8:5, and 3.6:0.4:5, with the remainder of the solution comprising a solvent such as acetone. Thus, for example, in a solution having a weight ratio of CA:PEG 3350:water of 2.8:1.2:5, CA comprises 2.8 wt % of the solution, PEG 3350 comprises 1.2 wt % of the solution, water comprises 5 wt % of the solution, and acetone comprises the remaining 91 wt %. Likewise, coating solution weight ratios of CA:HPC:water are 1.2:0.8:9.8, 2.4:1.6:19.6, 1.6:0.4:4.9, and 3.2:0.8:9.8, with the remainder of the solution comprising a solvent such as acetone. Thus, for example, in a solution having a weight ratio of CA:HPC:water of 1.2:0.8:10, CA comprises 1.2 wt % of the solution, HPC comprises 0.8 wt % of the solution, water comprises 10 wt % of the solution, and acetone comprises the remaining 88 wt %. Further, coating solution weight ratios of CA:HPC:methanol are 1.8:1.2:19.6, 2.4:1.6:19.6, 1.6:0.4:4.9, and 3.2:0.8:9.8, with the remainder of the solution comprising a solvent such as acetone. Thus, for example, in a solution having a weight ratio of CA:HPC:methanol of 1.8:1.2:19.6, CA comprises 1.8 wt % of the solution, HPC comprises 1.2 wt % of the solution, methanol comprises 19.6 wt % of the solution, and acetone comprises the remaining 77.4 wt %.

When incorporating antioxidants into the coating solution, a third solvent may be required to ensure good dispersion of the antioxidant into the coating. For example, a CA:PEG:water composition of 2.4:1.6:5 that includes 0.05 wt % wt of the solution requires 5 wt % methanol and 86% acetone.

Coatings formed from these coating solutions are generally porous. By “porous” is meant that the coating in the dry state has a density less than the density of the same material in a nonporous form. By “nonporous form” is meant a coating material formed by using a coating solution containing no non-solvent, or the minimal amount of non-solvent required to produce a homogeneous coating solution. The dry-state density of the coating can be calculated by dividing the coating weight (determined from the weight gain of the tablets before and after coating) by the coating volume (calculated by multiplying the coating thickness, as determined by optical or scanning electron microscopy, by the tablet surface area). The porosity of the coating is one of the factors that leads to the combination of high water permeability and high strength of the coating.

While porous coatings based on CA, PEG or HPC, and water described above translate to excellent results, other pharmaceutically acceptable materials could be used in the coating so long as the coating has the requisite combination of high water permeability, high strength, and ease of fabrication and application. Further, such coatings may be dense, porous, or “asymmetric,” having one or more dense layers and one or more porous layers such as those disclosed in U.S. Pat. Nos. 5,612,059 and 5,698,220, the pertinent disclosures of which are incorporated herein by reference.

The coating must also contain at least one delivery port in communication with the interior and exterior of the coating to allow for release of the drug-containing composition to the exterior of the dosage form. The delivery port can range in size from about the size of the drug particles, and thus could be as small as 1 to 100 microns in diameter and may be termed pores, up to about 5000 microns in diameter. The shape of the port may be substantially circular, in the form of a slit, or other convenient shape to ease manufacturing and processing. The port(s) may be formed by post-coating mechanical or thermal means or with a beam of light (e.g., a laser), a beam of particles, or other high-energy source, or may be formed in situ by rupture of a small portion of the coating. Such rupture may be controlled by intentionally incorporating a relatively small weak portion into the coating. Delivery ports may also be formed in situ by erosion of a plug of water-soluble material or by rupture of a thinner portion of the coating over an indentation in the core. Delivery ports may be formed by coating the core such that one or more small regions remain uncoated. In addition, the delivery port can be a large number of holes or pores that may be formed during coating, as in the case of asymmetric membrane coatings, described in more detail herein, and of the type disclosed in U.S. Pat. Nos. 5,612,059 and 5,698,220, the disclosures of which are incorporated by reference. When the delivery pathways are pores there can be a multitude of such pores that range in size from 1 micron to greater than 100 microns. During operation, one or more of such pores may enlarge under the influence of the hydrostatic pressure generated during operation. At least one delivery port should be formed on the side of coating that is adjacent to the 1-(((2S,3S,4S)-3-ethyl-4-fluoro-5-oxopyrrolidin-2-yl)methoxy)-7-methoxyisoquinoline-6-carboxamide-containing composition, so that the 1-(((2S,3S,4S)-3-ethyl-4-fluoro-5-oxopyrrolidin-2-yl)methoxy)-7-methoxyisoquinoline-6-carboxamide-containing composition will be extruded out of the delivery port by the swelling action of the water-swellable composition. It is recognized that some processes for forming delivery ports may also form holes or pores in the coating adjacent to the water-swellable composition.

The coating may optionally include a port in communication with the water-swellable composition. Such a delivery port does not typically alter the 1-(((2S,3S,4S)-3-ethyl-4-fluoro-5-oxopyrrolidin-2-yl)methoxy)-7-methoxyisoquinoline-6-carboxamide release characteristics of the dosage form, but may provide manufacturing advantages. It is believed that the water-swellable compositions, such as those containing PEO with a molecular weight between 3,000,000 and 8,000,000 daltons, are too viscous to appreciably exit the port. In dosage forms wherein the delivery ports are drilled either mechanically or by laser, the tablet must be oriented so that at least one delivery port is formed in the coating adjacent to the 1-(((2S,3S,4S)-3-ethyl-4-fluoro-5-oxopyrrolidin-2-yl)methoxy)-7-methoxyisoquinoline-6-carboxamide-containing composition. A colorant within the water-swellable composition is used to orient the core dosage form during the drilling step in manufacture. By providing a delivery port on both faces of the dosage form, the need to orient the dosage form may be eliminated and the colorant may be removed from the water-swellable composition.

In yet another embodiment, 1-(((2S,3S,4S)-3-ethyl-4-fluoro-5-oxopyrrolidin-2-yl)methoxy)-7-methoxyisoquinoline-6-carboxamide is incorporated into a variation of the above disclosed osmotic delivery device, an asymmetric membrane technology (AMT). These devices have been disclosed in Herbig, et al., J. Controlled Release, 35, 1995, 127-136, and U.S. Pat. Nos. 5,612,059 and 5,698,220 as coatings in osmotic drug delivery systems. These AMT systems provide the general advantages of osmotic controlled release devices (reliable drug delivery independent of position in gastrointestinal tract), yet do not require the added manufacturing step of drilling a hole in the coating, as seen with a number of other osmotic systems. In the formation of these porous coatings, a water-insoluble polymer is combined with a water-soluble, pore-forming material. The mixture is coated onto an osmotic tablet core from a combination of water and solvent. As the coating dries, a phase inversion process occurs whereby a porous, asymmetric membrane is produced. The use of an AMT system for controlled release of a drug with similar physiochemical properties is described in US patent Application Publication US2007/0248671 and herein incorporated as reference.

While a number of materials have been disclosed for use as pore-formers in the production of asymmetric membranes, the previously disclosed materials all bring chemical or physical stability issues into the system. In particular, many of the prior art materials are liquids, which can potentially migrate out of the coating during storage. Of the ones that are solid, both polymeric materials and inorganic materials have been taught. Inorganic materials can be difficult to use for a number of reasons. In particular, they often have a tendency to crystallize and/or adsorb moisture on storage. The particular polymeric materials that have been taught include polyvinylpyrrolidone (PVP) and polyethylene glycol (PEG) derivatives. Both of these materials have a strong tendency to form peroxides and/or formaldehyde upon storage (see for example Waterman, et al., “Impurities in Drug Products” in Handbook of Isolation and Characterization of Impurities in Pharmaceuticals, S. Ajira and K. M. Alsante, Eds. 2003, pp. 75-85). Many drug substances are reactive with such polymer degradation products, both because of their intrinsic reactivity and their tendency to migrate upon storage. However, this formulation space is relatively narrow. U.S. Pat. No. 4,519,801 discloses a wide list of water-soluble polymeric components useful for coatings in osmotic systems, but fails to teach appropriate selections of water-soluble components for AMT systems. There remains, therefore, a need for new pore-forming materials for AMT systems wherein the pore-forming materials do not generate reactive byproducts, crystallize or migrate from the coating upon storage.

In another embodiment the present invention provides a dosage form which comprises (a) a core containing at least one pharmaceutically active ingredient and (b) at least one asymmetric membrane technology coating wherein said coating comprises:

a. one or more substantially water-insoluble polymers, and

b. one or more solid, water-soluble polymeric materials that do not contain amounts of hydrogen peroxide or formaldehyde greater than about 0.01 percent w:w after storage at 40 degrees C./75 percent RH for 12 weeks.

One aspect of the present invention also provides a dosage form wherein the dosage form delivers drug primarily by osmotic pressure. In particular embodiments, the present invention provides a dosage form wherein the pharmaceutically active ingredient is 1-(((2S,3S,4S)-3-ethyl-4-fluoro-5-oxopyrrolidin-2-yl)methoxy)-7-methoxyisoquinoline-6-carboxamide or a pharmaceutically acceptable salt thereof. The water-insoluble polymer as used in the present invention preferably comprises a cellulose derivative, more preferably, cellulose acetate. The solid, water-soluble polymeric material comprises a polymer having a weight average molecular weight between 2000 and 50,000 daltons. The solid, water-soluble polymeric material is selected from the group consisting of water-soluble cellulose derivatives, acacia, dextrin, guar gum, maltodextrin, sodium alginate, starch, polyacrylates, polyvinyl alcohols and zein. The water-soluble cellulose derivatives comprise hydroxypropylcellulose, hydroxypropylmethylcellulose and hydroxyethylcellulose. The solid, water-soluble, polymeric material has a viscosity for a 5 percent w:w aqueous solution of less than 400 mPa s. The solid, water-soluble, polymeric material has a viscosity for a 5 percent w:w aqueous solution of less than 300 mPa s. In other embodiments, the solid, water-soluble, polymeric material has a softening temperature greater than 55 degrees C.

A process of the present invention encompasses the process wherein the coating is applied from a mixture of acetone and water using a pan coating. The process of the present invention also encompasses the process wherein the asymmetric membrane comprises cellulose acetate and hydroxypropylcellulose which is coated from a mixture of acetone to water between about 9:1 and 6:4, w:w, and more preferably between about 7:3 and about 6:4, w:w, using a pan coater. In particular, the process of the present invention encompasses the process wherein the core comprises 1-(((2S,3S,4S)-3-ethyl-4-fluoro-5-oxopyrrolidin-2-yl)methoxy)-7-methoxyisoquinoline-6-carboxamide, or a pharmaceutically acceptable salt thereof.

In the preparation of the asymmetric membrane coatings of the present invention, the water-insoluble component of the asymmetric membrane coating preferentially is formed from cellulose derivatives. In particular, these derivatives include cellulose esters and ethers, namely the mono-, di- and triacyl esters wherein the acyl group consists of two to four carbon atoms and lower alkyl ethers of cellulose wherein the alkyl group has one to four carbon atoms. The cellulose esters can also be mixed esters, such as cellulose acetate butyrate, or a blend of cellulose esters. The same variations can be found in ethers of cellulose and include blends of cellulose esters and cellulose ethers. Other cellulose derivatives which can be used in making asymmetric membranes of the present invention include cellulose nitrate, acetaldehyde dimethyl cellulose, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate p-toluene sulfonate, cellulose cyanoacetates, cellulose acetate trimellitate, cellulose methacrylates and hydroxypropylmethylcellulose acetate succinate. A particularly preferred water-insoluble component is cellulose acetate. Particularly preferred cellulose acetates include those having an acetyl content of about 40 percent and a hydroxyl content of about 3.5 percent. Other materials also can be used in the fabrication of asymmetric membrane technology coatings, provided such materials are substantially water-insoluble, film-forming and safe to use in pharmaceutical applications.

In the preparation of the asymmetric membrane coatings of the present invention, the water-soluble polymeric component of the present invention comprises solid, polymeric materials that do not form hydrogen peroxide or formaldehyde upon storage for 12 weeks at 40 degrees C./75 percent relative humidity, in an amount greater than about 0.01 percent w/w (100 parts per million, ppm). In terms of water solubility, the solid polymeric water-soluble material preferentially has a water-solubility of greater than 0.5 mg/mL; more preferably, greater than 2 mg/mL; and still more preferably, greater than 5 mg/mL.

The solid polymeric water-soluble material has a melting or softening temperature above room temperature. Preferentially, the solid material has a melting or softening temperature above 30 degrees C.; more preferentially, above 40 degrees C.; and most preferentially, above 50 degrees C. Melting and softening points can be determined visually using a melting point apparatus, or alternatively, can be measured using differential scanning calorimetry (DSC), as is known in the art. The polymer can be either a homopolymer or a copolymer. Such polymers can be natural polymers, or be derivatives of natural products, or be entirely synthetic. The molecular weight of such materials is preferentially high enough to prevent migration and aid in film-forming, yet low enough to allow coating (as discussed below). The preferred molecular weight range for the present invention is therefore between 2000 and 50,000 daltons (weight average). Preferred polymers suitable as water-soluble components of an asymmetric membrane technology coating for the present invention include substituted, water-soluble cellulose derivatives, acacia, dextrin, guar gum, maltodextrin, sodium alginate, starch, polyacrylates, polyvinyl alcohols and zein. Particularly preferred water-soluble polymers include hydroxyethylcellulose, hydroxypropylcellulose and polyvinylalcohol.

It is difficult to obtain asymmetric membrane coatings if the viscosity of the coating solution is too high, and that one approach to solving this issue is to use more dilute solutions of the polymer. Due to the phase behavior of the coating solution, having both water-soluble and organic-soluble components, there is a limit to how low the concentration of the water-soluble polymer can be and still provide a commercializable process. For this reason, it is preferred that the water-soluble polymers not have too high a viscosity. Viscosities can be determined at 25 degrees C. using a Brookfield LVF viscometer (available from Brookfield Engineering Corp., Middleboro, Mass.) with spindle and speed combinations depending on viscosity levels for 5 percent (w:w) aqueous solutions. Preferred water-soluble polymers have viscosities for 5 percent (w:w) solutions of less than 400 mPa s; more preferably, less than 300 mPa s.

Using the above criteria, especially preferred water-soluble polymers include hydroxypropylcellulose and hydroxyethylcellulose having a viscosity for a 5 percent (w:w) of less than 300 mPa s. Commercially available examples of such polymers include Klucel EF™ and Natrasol LR™, both made by the Aqualon Division of Hercules Corp., Hopewell, Va.

The water-soluble, solid polymeric material's stability to formation of hydrogen peroxide can be measured by storing the polymer in an oven having a temperature and relative humidity (RH) of 40 degrees C. and 75 percent RH, respectively. The polymer should be stored exposed to the oven environment under “open” conditions. The polymer should be stored for at least 12 weeks. Levels of hydrogen peroxide can be administered as described in G. M. Eisenberg, “Colorimetric determination of hydrogen peroxide” in Ind. Eng. Chem. (Anal. Ed.), 1943, 15, 327-328. Under these storage conditions, acceptable polymeric materials for the present invention have hydrogen peroxide levels below 100 parts per million (ppm); more preferably, below 50 ppm; and most preferably, below 10 ppm.

Similarly, the water-soluble polymer's stability to formation of formaldehyde can be measured by storing the polymer in an oven at 40 degrees C. and 75 percent RH. Polymer should be stored in a sealed container to avoid loss of volatile formaldehyde. The polymer should be stored for at least 12 weeks. Levels of formaldehyde can be determined as described in M. Ashraf-Khorassani, et al., “Purification of pharmaceutical excipients with supercritical fluid extraction” in Pharm. Dev. Tech. 2005, 10, 1-10. Under these storage conditions, acceptable water-soluble polymeric materials for the present invention have formaldehyde levels below 100 ppm, more preferably, below 50 ppm, and most preferably, below 10 ppm.

It will be appreciated by those skilled in the art that the asymmetric membrane technology coating formulation can contain small amounts of other materials without significantly changing its function or altering the nature of the present invention. Such additives include glidants (e.g., talc and silica) and plasticizers (e.g., triethylcitrate and triacetin), which are typically added, when needed, at levels of less than about 5 percent (w:w) of the coating.

It will be appreciated by those skilled in the art that active pharmaceutical ingredients can also be in the form of pharmaceutically acceptable salts. The cores for the present invention can also employ solubilizing additives. Such additives include pH-buffering additives to maintain the core at a pH wherein the active pharmaceutical ingredient has a sufficiently high solubility to be pumped out of the dosage form in solution.

The core can contain osmotic agents which help to provide the driving force for drug delivery. Such osmotic agents include water-soluble sugars and salts. Particularly preferred osmotic agents are dextrates and sodium chloride.

The core of the AMT system can contain other additives to provide for such benefits as stability, manufacturability and system performance. Stabilizing excipients include pH-modifying ingredients, antioxidants, chelating agents, and other such additives as is known in the art. Excipients that improve manufacturability include agents to help in flow, compression or extrusion. Flow can be helped by such additives as talc, stearates and silica. Flow is also improved by granulation of the drug and excipients, as is known in the art. Such granulations often benefit from the addition of binders such as hydroxypropylcellulose, starch and polyvinylpyrollidone (povidone). Compression can be improved by the addition of diluents to the formulation. Examples of diluents include lactose, mannitol, microcrystalline cellulose and the like, as is known in the art. For cores produced by extrusion, the melt properties of the excipients can be important. Generally, it is preferable that such excipients have melting temperatures below about 100 degrees C. Examples of appropriate excipients for melt processes include esterified glycerines and stearyl alcohol. For compressed dosage forms, manufacturability can be improved by addition of lubricants. Preferred lubricants are magnesium stearate and sodium stearyl fumarate.

Cores can be produced using standard tablet compression processes, as is known in the art. Such processes involve powders filling dies followed by compression using appropriate punches. Cores can also be produced by an extrusion process. Extrusion processes are especially well-suited to making small cores (multiparticulates). A preferred extrusion process is a melt-spray-congeal process as described in WO2005/053653A1, incorporated by reference. Cores can also be prepared by layering drug onto seed cores. Such seed cores are preferentially made of sugar or microcrystalline cellulose. Drug can be applied onto the cores by spraying, preferentially in a fluid-bed operation, as is known in the art.

In the practice of the subject invention, the cores are coated with the asymmetric membrane by any technique that can provide the asymmetric membrane as a coating over the entire cores. Preferred coating methods include pan coating and fluid-bed coating. In both coating processes, the water-insoluble polymer and water-soluble polymer as well as any other additives are first dissolved or dispersed in an appropriate solvent or solvent combination. In order to achieve a suitably porous membrane, the coating solvent needs to be optimized for performance. Generally, the solvents are chosen such that the more volatile solvent is the better solvent for the water-insoluble polymeric component. The result is that during coating, the water-insoluble polymeric component precipitates from solution. Preferred solvents and solvent ratios can be determined by examining the multi-component solubility behavior of the system.

In another embodiment of the present invention, 1-(((2S,3S,4S)-3-ethyl-4-fluoro-5-oxopyrrolidin-2-yl)methoxy)-7-methoxyisoquinoline-6-carboxamide is incorporated into a monolithic osmotic delivery device, known as an extrudable core system, such that the 1-(((2S,3S,4S)-3-ethyl-4-fluoro-5-oxopyrrolidin-2-yl)methoxy)-7-methoxyisoquinoline-6-carboxamide-containing composition may include viscosifying polymers and osmoticallly active agents, and may optionally include solubility enhancing agents and/or antioxidants. The monolithic tablet or capsule is surrounded by a semi-permeable membrane which contains one or more openings which are manufactured into the dosage form through such techniques as laser drilling. The viscosifying polymers suspend or entrain the drug so as to aid in the delivery of the drug through the delivery port(s). While not wishing to be bound by any particular theory, it is believed that upon the imbibition of water into the dosage form, the viscosifying polymer has enough viscosity to allow it to suspend or entrain the drug, while at the same time remaining sufficiently fluid to allow the viscosifying polymer to pass through the delivery port(s) along with the drug. The viscosifying polymer may be a single material or a mixture of materials. Non-crosslinked polyethylene oxide (PEO) and Hydroxyethyl cellulose (HEC) may be used as the viscosifying polymers.

The 1-(((2S,3S,4S)-3-ethyl-4-fluoro-5-oxopyrrolidin-2-yl)methoxy)-7-methoxyisoquinoline-6-carboxamide-containing composition also includes osmotically effective solutes, often referred to as “osmogens” or “osmagents.” Typical classes of suitable osmogens are water-soluble salts, sugars, organic acids, and other low-molecule-weight organic compounds that are capable of imbibing water to thereby establish an osmotic pressure gradient across the barrier of the surrounding coating. Typical useful salts include magnesium sulfate, magnesium chloride, calcium chloride, sodium chloride, lithium chloride, potassium sulfate, sodium carbonate, sodium sulfite, lithium sulfate, potassium chloride, sodium sulfate and dextrates. A preferred salt is sodium chloride. Preferred organic acids include ascorbic acid, 2-benzene carboxylic acid, benzoic acid, fumaric acid, citric acid, maleic acid, serbacic acid, sorbic acid, edipic acid, editic acid, glutamic acid, toluene sulfonic acid, and tartaric acid. Preferred sugars include mannitol, sucrose, sorbitol, xylitol, lactose, dextrose, and trehlaose. The osmogens can be used alone or as a combination of two or more osmogens.

The 1-(((2S,3S,4S)-3-ethyl-4-fluoro-5-oxopyrrolidin-2-yl)methoxy)-7-methoxyisoquinoline-6-carboxamide-containing composition may further include solubility-enhancing agents or solubilizers that promote the aqueous solubility of the drug. Solubilizers useful with 1-(((2S,3S,4S)-3-ethyl-4-fluoro-5-oxopyrrolidin-2-yl)methoxy)-7-methoxyisoquinoline-6-carboxamide include organic acids and organic acid salts, partial glycerides, e.g., less than fully esterified derivatives of glycerin, including glycerides, monoglycerides, diglycerides, glyceride derivatives, polyethylene glycol esters, polypropylene glycol esters, polyhydric alcohol esters, polyoxyethylene ethers, sorbitan esters, polyoxyethylene sorbitan esters, and carbonate salts.

A preferred class of solubilizers is organic acids. Since 1-(((2S,3S,4S)-3-ethyl-4-fluoro-5-oxopyrrolidin-2-yl)methoxy)-7-methoxyisoquinoline-6-carboxamide is a base which is solubilized by protonation, it is believed that addition of an organic acid to the 1-(((2S,3S,4S)-3-ethyl-4-fluoro-5-oxopyrrolidin-2-yl)methoxy)-7-methoxyisoquinoline-6-carboxamide-containing composition assists in solubilization and henc absorption of 1-(((2S,3S,4S)-3-ethyl-4-fluoro-5-oxopyrrolidin-2-yl)methoxy)-7-methoxyisoquinoline-6-carboxamide. Organic acids can also promote stability during storage prior to introduction to a use environment due to their tendency to maintain 1-(((2S,3S,4S)-3-ethyl-4-fluoro-5-oxopyrrolidin-2-yl)methoxy)-7-methoxyisoquinoline-6-carboxamide in a protonated state.

There are a variety of factors to consider when choosing an appropriate organic acid for use as a solubilizer with 1-(((2S,3S,4S)-3-ethyl-4-fluoro-5-oxopyrrolidin-2-yl)methoxy)-7-methoxyisoquinoline-6-carboxamide in an osmotic dosage form. The acid should not interact adversely with 1-(((2S,3S,4S)-3-ethyl-4-fluoro-5-oxopyrrolidin-2-yl)methoxy)-7-methoxyisoquinoline-6-carboxamide, should have appropriate water solubility, and should provide good manufacturing properties.

The 1-(((2S,3S,4S)-3-ethyl-4-fluoro-5-oxopyrrolidin-2-yl)methoxy)-7-methoxyisoquinoline-6-carboxamide-containing composition layer and/or the functional rate controlling membrane may optionally contain an antioxidant, such as but not limited to BHT, BHA, sodium metabisulfite, propyl galate, glycerin, vitamin E, Citric Acid or ascorbyl palmitate. The antioxidant may be present in an amount ranging from 0 to 10 wt % of the 1-(((2S,3S,4S)-3-ethyl-4-fluoro-5-oxopyrrolidin-2-yl)methoxy)-7-methoxyisoquinoline-6-carboxamide-containing composition layer and/or the water-swellable composition layer and/or the functional rate controlling membrane. For additional examples of antioxidants, see C.-M. Andersson, A. Hallberg, and T. Hoegberg. Advances in the development of pharmaceutical antioxidants. Advances in Drug Research. 28:65-180, 1996.

The 1-(((2S,3S,4S)-3-ethyl-4-fluoro-5-oxopyrrolidin-2-yl)methoxy)-7-methoxyisoquinoline-6-carboxamide-containing composition is prepared by mixing the viscosifying polymer and the other excipients to form a uniform blend. To obtain a uniform blend, it is desirable to either wet or dry granulate or dry blend the components using the types of processes known to those skilled in the art.

### Tableting

The core is prepared by first placing a mixture of the 1-(((2S,3S,4S)-3-ethyl-4-fluoro-5-oxopyrrolidin-2-yl)methoxy)-7-methoxyisoquinoline-6-carboxamide-containing composition into a tablet press and compressed in order to complete formation of the core. Tablet shapes may include any tablet shape known to those skilled in the art. Preferable tablet shapes include SRC (standard round concave), oval, modified oval, capsule, caplet, and almond. More preferable tablet shapes include oval, modified oval, caplet, and capsule.

Following formation of the core, the semi-permeable coating is applied. The coating should have high water permeability and a high strength, while at the same time be easily fabricated and applied. High water permeability is required to permit water to enter the core in sufficient volume. High strength is required to ensure the coating does not burst when the core swells as it imbibes water, leading to an uncontrolled delivery of the core contents. Finally, the coating must have high reproducibility and yield.

It is essential that the coating have at least one delivery port in communication with the interior and exterior of the coating for delivery of the 1-(((2S,3S,4S)-3-ethyl-4-fluoro-5-oxopyrrolidin-2-yl)methoxy)-7-methoxyisoquinoline-6-carboxamide-containing composition.

Furthermore, the coating must be non-dissolving and non-eroding during release of the 1-(((2S,3S,4S)-3-ethyl-4-fluoro-5-oxopyrrolidin-2-yl)methoxy)-7-methoxyisoquinoline-6-carboxamide-containing composition, generally meaning that it be water-insoluble, such that 1-(((2S,3S,4S)-3-ethyl-4-fluoro-5-oxopyrrolidin-2-yl)methoxy)-7-methoxyisoquinoline-6-carboxamide is substantially entirely delivered through the delivery port(s), in contrast to delivery via permeation through the coating.

Coatings with these characteristics can be obtained using hydrophilic polymers such as plasticized and unplasticized cellulose esters, ethers, and ester-ethers. Particularly suitable polymers include cellulose acetate (CA), cellulose acetate butyrate (CAB), and ethyl cellulose (EC). One set of polymers are cellulose acetates having acetyl contents of 25 to 42%. One typical polymer is CA having an acetyl content of 39.8%, specifically, CA 398-10 (Eastman Fine Chemicals, Kingsport, Tenn.). CA 398-10 is reported to have an average molecular weight of about 40,000 daltons. Another typical CA having an acetyl content of 39.8% is high molecular weight CA having an average molecular weight greater than about 45,000, and specifically, CA 398-30 (Eastman Fine Chemical) which is reported to have an average molecular weight of 50,000 daltons.

Coating is conducted in conventional fashion by first forming a coating solution and then coating by dipping, fluidized bed coating, or by pan coating. To accomplish this, a coating solution is formed comprising the polymer and a solvent. Typical solvents useful with the cellulosic polymers above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, and mixtures thereof. The coating solution typically contains 2 to 15 wt % of the polymer.

The coating solution may also include pore-formers or non-solvents in any amount as long as the polymer remains soluble at the conditions used to form the coating and as long as the coating remains water permeable and has sufficient strength. Pore-formers and their use in fabricating coatings are described in U.S. Pat. Nos. 5,698,220 and 5,612,059, the pertinent disclosures of which are incorporated herein by reference. The term “pore former,” as used herein, refers to a material added to the coating solution that has low or no volatility relative to the solvent such that it remains as part of the coating following the coating process but that is sufficiently water swellable or water soluble such that, in the aqueous use environment it provides a water-filled or water-swollen channel or “pore” to allow the passage of water, thereby enhancing the water permeability of the coating. Suitable pore formers include but are not limited to hydroxypropylcellulose (HPC), polyethylene glycol (“PEG”), PVP, and PEO. To obtain a combination of high water permeability and high strength when PEG or HPC are used as a pore former, the weight ratio of CA:PEG or CA:HPC should range from about 6:4 to about 9:1. CA:HPC is a preferred coating composition. Preferred CA:HPC weight ratios should range from 6:4 to 7:3. Preferred CA:PEG weight ratios should range from 6:4 to 7:3.

The addition of a non-solvent such as water or methanol to the coating solution results in exceptional performance. By “non-solvent” is meant any material added to the coating solution that substantially dissolves in the coating solution and reduces the solubility of the coating polymer or polymers in the solvent. In general, the function of the non-solvent is to impart porosity to the resulting coating. As described below, porous coatings have higher water permeability than an equivalent weight of a coating of the same composition that is not porous and this porosity is indicated by a reduction in the density of the coating (mass/volume). Although not wishing to be bound by any particular mechanism of pore formation, it is generally believed that addition of a non-solvent imparts porosity to the coating during evaporation of solvent by causing the coating solution to undergo liquid and liquid phase separation prior to solidification. The suitability and amount of a particular candidate material can be evaluated for use as a non-solvent by progressively adding the candidate non-solvent to the coating solution until it becomes cloudy. If this does not occur at any addition level up to about 50 wt % of the coating solution, it generally is not appropriate for use as a non-solvent. When clouding is observed, termed the “cloud point,” an appropriate level of non-solvent for maximum porosity is the amount just below the cloud point. For acetone solutions comprising 7 wt % CA and 3 wt % PEG, the cloud point is at about 23 wt % water. When lower porosities are desired, the amount of non-solvent can be reduced as low as desired.

Suitable non-solvents are any materials that have appreciable solubility in the solvent and that lower the coating polymer solubility in the solvent. The preferred non-solvent depends on the solvent and the coating polymer chosen. In the case of using a volatile polar coating solvent such as acetone, suitable non-solvents include water, glycerol, alcohols such as methanol or ethanol.

When using CA 398-10, coating solution weight ratios of CA:PEG 3350: water are 2.4:1.6:5, 2.8:1.2:5, 3.2:0.8:5, and 3.6:0.4:5, with the remainder of the solution comprising a solvent such as acetone. Thus, for example, in a solution having a weight ratio of CA:PEG 3350: water of 2.8:1.2:5, CA comprises 2.8 wt % of the solution, PEG 3350 comprises 1.2 wt % of the solution, water comprises 5 wt % of the solution, and acetone comprises the remaining 91 wt %. Likewise, coating solution weight ratios of CA:HPC:water are 1.2:0.8:9.8, 2.4:1.6:19.6, 1.6:0.4:4.9, and 3.2:0.8:9.8, with the remainder of the solution comprising a solvent such as acetone. Thus, for example, in a solution having a weight ratio of CA:HPC:water of 1.2:0.8:10, CA comprises 1.2 wt % of the solution, HPC comprises 0.8 wt % of the solution, water comprises 10 wt % of the solution, and acetone comprises the remaining 88 wt %. Further, coating solution weight ratios of CA:HPC:methanol are 1.8:1.2:19.6, 2.4:1.6:19.6, 1.6:0.4:4.9, and 3.2:0.8:9.8, with the remainder of the solution comprising a solvent such as acetone. Thus, for example, in a solution having a weight ratio of CA:HPC:methanol of 1.8:1.2:19.6, CA comprises 1.8 wt % of the solution, HPC comprises 1.2 wt % of the solution, methanol comprises 19.6 wt % of the solution, and acetone comprises the remaining 77.4 wt %.

When incorporating antioxidants into the coating solution, a third solvent may be required to ensure good dispersion of the antioxidant into the coating. For example, a CA:PEG:water composition of 2.4:1.6:5 that includes 0.05 wt % wt of the solution requires 5 wt % methanol and 86% acetone.

Coatings formed from these coating solutions are generally porous. By “porous” is meant that the coating in the dry state has a density less than the density of the same material in a nonporous form. By “nonporous form” is meant a coating material formed by using a coating solution containing no non-solvent, or the minimal amount of non-solvent required to produce a homogeneous coating solution. The dry-state density of the coating can be calculated by dividing the coating weight (determined from the weight gain of the tablets before and after coating) by the coating volume (calculated by multiplying the coating thickness, as determined by optical or scanning electron microscopy, by the tablet surface area). The porosity of the coating is one of the factors that leads to the combination of high water permeability and high strength of the coating.

While porous coatings based on CA, PEG or HPC, and water or methanol described above translate to excellent results, other pharmaceutically acceptable materials could be used in the coating so long as the coating has the requisite combination of high water permeability, high strength, and ease of fabrication and application. Further, such coatings may be dense, porous, or “asymmetric,” having one or more dense layers and one or more porous layers such as those disclosed in U.S. Pat. Nos. 5,612,059 and 5,698,220, the pertinent disclosures of which are incorporated herein by reference.

The coating must also contain at least one delivery port in communication with the interior and exterior of the coating to allow for release of the tablet core contents to the exterior of the dosage form. The delivery port can range in size from about the size of the drug particles, and thus could be as small as 1 to 100 microns in diameter and may be termed pores, up to about 5000 microns in diameter. The shape of the port may be substantially circular, in the form of a slit, or other convenient shape to ease manufacturing and processing. The port(s) may be formed by post-coating mechanical or thermal means or with a beam of light (e.g., a laser), a beam of particles, or other high-energy source, or may be formed in situ by rupture of a small portion of the coating. Such rupture may be controlled by intentionally incorporating a relatively small weak portion into the coating. Delivery ports may also be formed in situ by erosion of a plug of water-soluble material or by rupture of a thinner portion of the coating over an indentation in the core. Delivery ports may be formed by coating the core such that one or more small regions remain uncoated. In addition, the delivery port can be a large number of holes or pores that may be formed during coating, as in the case of asymmetric membrane coatings, described in more detail herein, and of the type disclosed in U.S. Pat. Nos. 5,612,059 and 5,698,220, the disclosures of which are incorporated by reference. When the delivery pathways are pores there can be a multitude of such pores that range in size from 1 micron to greater than 100 microns. During operation, one or more of such pores may enlarge under the influence of the hydrostatic pressure generated during operation. The location of the delivery port(s) may be located anywhere on the tablet surface. Preferred locations of the delivery port(s) include the face of the tablet and the tablet band. A more preferred location includes approximately the center of the tablet band for round, SRC-shaped tablets and approximately the center of the tablet band along the major axis and/or approximately the center of the tablet band along the minor axis of the tablet band for capsule, caplet, oval, or modified oval shaped tablets. A most preferred location of the delivery port(s) is the approximate center of the tablet band along the major axis of the tablet band for capsule, caplet, oval, or modified oval shaped tablets.

Another class of 1-(((2S,3S,4S)-3-ethyl-4-fluoro-5-oxopyrrolidin-2-yl)methoxy)-7-methoxyisoquinoline-6-carboxamide sustained-release dosage forms of this invention includes membrane-moderated or reservoir systems. In this class, a reservoir of 1-(((2S,3S,4S)-3-ethyl-4-fluoro-5-oxopyrrolidin-2-yl)methoxy)-7-methoxyisoquinoline-6-carboxamide is surrounded by a rate-limiting membrane. The 1-(((2S,3S,4S)-3-ethyl-4-fluoro-5-oxopyrrolidin-2-yl)methoxy)-7-methoxyisoquinoline-6-carboxamide traverses the membrane by mass transport mechanisms well known in the art, including but not limited to dissolution in the membrane followed by diffusion across the membrane or diffusion through liquid-filled pores within the membrane.

These individual reservoir system dosage forms may be large, as in the case of a tablet containing a single large reservoir, or multiparticulate, as in the case of a capsule containing a plurality of reservoir particles, each individually coated with a membrane. The coating can be non-porous, yet permeable to 1-(((2S,3S,4S)-3-ethyl-4-fluoro-5-oxopyrrolidin-2-yl)methoxy)-7-methoxyisoquinoline-6-carboxamide (for example 1-(((2S,3S,4S)-3-ethyl-4-fluoro-5-oxopyrrolidin-2-yl)methoxy)-7-methoxyisoquinoline-6-carboxamide may diffuse directly through the membrane), or it may be porous.

Sustained release coatings as known in the art may be employed to fabricate the membrane, especially polymer coatings, such as a cellulose ester or ether, an acrylic polymer, or a mixture of polymers. Preferred materials include ethyl cellulose, cellulose acetate and cellulose acetate butyrate. The polymer may be applied as a solution in an organic solvent or as an aqueous dispersion or latex. The coating operation may be conducted in standard equipment such as a fluid bed coater, a Wurster coater, or a rotary bed coater.

If desired, the permeability of the coating may be adjusted by blending of two or more materials. A useful process for tailoring the porosity of the coating comprises adding a pre-determined amount of a finely-divided water-soluble material, such as sugars or salts or water-soluble polymers to a solution or dispersion (e.g., an aqueous latex) of the membrane-forming polymer to be used. When the dosage form is ingested into the aqueous medium of the GI tract, these water soluble membrane additives are leached out of the membrane, leaving pores which facilitate release of the drug. The membrane coating can also be modified by the addition of plasticizers, as known in the art.

A useful variation of the process for applying a membrane coating comprises dissolving the coating polymer in a mixture of solvents chosen such that as the coating dries, a phase inversion takes place in the applied coating solution, resulting in a membrane with a porous structure. Numerous examples of this type of coating system are given in European Patent Specification 0 357 369 B1, published Mar. 7, 1990, herein incorporated by reference.

The morphology of the membrane is not of critical importance so long as the permeability characteristics enumerated herein are met. The membrane can be amorphous or crystalline. It can have any category of morphology produced by any particular process and can be, for example, an interfacially-polymerized membrane (which comprises a thin rate-limiting skin on a porous support), a porous hydrophilic membrane, a porous hydrophobic membrane, a hydrogel membrane, an ionic membrane, and other such materials which are characterized by controlled permeability to t1-(((2S,3S,4S)-3-ethyl-4-fluoro-5-oxopyrrolidin-2-yl)methoxy)-7-methoxyisoquinoline-6-carboxamide.

A useful reservoir system embodiment is a capsule having a shell comprising the material of the rate-limiting membrane, including any of the membrane materials previously discussed, and filled with a 1-(((2S,3S,4S)-3-ethyl-4-fluoro-5-oxopyrrolidin-2-yl)methoxy)-7-methoxyisoquinoline-6-carboxamide drug composition. A particular advantage of this configuration is that the capsule may be prepared independently of the drug composition, thus process conditions that would adversely affect the drug can be used to prepare the capsule. One embodiment is a capsule having a shell made of a porous or a permeable polymer made by a thermal forming process. Another embodiment is a capsule shell in the form of an asymmetric membrane; e.g., a membrane that has a thin skin on one surface and most of whose thickness is constituted of a highly permeable porous material. A process for preparation of asymmetric membrane capsules comprises a solvent exchange phase inversion, wherein a solution of polymer, coated on a capsule-shaped mold, is induced to phase-separate by exchanging the solvent with a-miscible non-solvent. Examples of asymmetric membranes useful in this invention are disclosed in the aforementioned European Patent Specification 0 357 369 B1.

Another embodiment of the class of reservoir systems comprises a multiparticulate wherein each particle is coated with a polymer designed to yield sustained release of 1-(((2S,3S,4S)-3-ethyl-4-fluoro-5-oxopyrrolidin-2-yl)methoxy)-7-methoxyisoquinoline-6-carboxamide. The multiparticulate particles each comprise 1-(((2S,3S,4S)-3-ethyl-4-fluoro-5-oxopyrrolidin-2-yl)methoxy)-7-methoxyisoquinoline-6-carboxamide and one or more excipients as needed for fabrication and performance. The size of individual particles, as previously mentioned, is generally between about 50 micron and about 3 mm, although beads of a size outside this range may also be useful. In general, the beads comprise 1-(((2S,3S,4S)-3-ethyl-4-fluoro-5-oxopyrrolidin-2-yl)methoxy)-7-methoxyisoquinoline-6-carboxamide and one or more binders. As it is generally desirable to produce dosage forms which are small and easy to swallow, beads which contain a high fraction of 1-(((2S,3S,4S)-3-ethyl-4-fluoro-5-oxopyrrolidin-2-yl)methoxy)-7-methoxyisoquinoline-6-carboxamide relative to excipients are preferred. Binders useful in fabrication of these beads include microcrystalline cellulose (e.g., Avicel®, FMC Corp.), hydroxypropyl cellulose (HPC), hydroxypropyl methyl cellulose (HPMC), and related materials or combinations thereof. In general, binders which are useful in granulation and tabletting, such as starch, pregelatinized starch, and poly (N-vinyl-2-pyrrolidinone) (PVP) may also be used to form multiparticulates.

Reservoir system 1-(((2S,3S,4S)-3-ethyl-4-fluoro-5-oxopyrrolidin-2-yl)methoxy)-7-methoxyisoquinoline-6-carboxamide multiparticulates may be prepared using techniques known to those skilled in the art, including, but not limited to, the techniques of extrusion and spheronization, wet granulation, fluid bed granulation, and rotary bed granulation. In addition, the beads may also be prepared by building the 1-(((2S,3S,4S)-3-ethyl-4-fluoro-5-oxopyrrolidin-2-yl)methoxy)-7-methoxyisoquinoline-6-carboxamide composition (drug plus excipients) up on a seed core (such as a non-pareil seed) by a drug-layering technique such as powder coating or by applying the 1-(((2S,3S,4S)-3-ethyl-4-fluoro-5-oxopyrrolidin-2-yl)methoxy)-7-methoxyisoquinoline-6-carboxamide composition by spraying a solution or dispersion of 1-(((2S,3S,4S)-3-ethyl-4-fluoro-5-oxopyrrolidin-2-yl)methoxy)-7-methoxyisoquinoline-6-carboxamide in an appropriate binder solution onto seed cores in a fluidized bed such as a Wurster coater or a rotary processor. An example of a suitable composition and method is to spray a dispersion of a 1-(((2S,3S,4S)-3-ethyl-4-fluoro-5-oxopyrrolidin-2-yl)methoxy)-7-methoxyisoquinoline-6-carboxamide/hydroxypropylcellulose composition in water. Advantageously, 1-(((2S,3S,4S)-3-ethyl-4-fluoro-5-oxopyrrolidin-2-yl)methoxy)-7-methoxyisoquinoline-6-carboxamide can be loaded in the aqueous composition beyond its solubility limit in water.

A method for manufacturing the multiparticulate cores of this embodiment is the extrusion/spheronization process, as previously discussed for matrix multiparticulates. Another process and composition for this method involves using water to wet-mass blend of about 5 to 75% of micro-crystalline cellulose with correspondingly about 95 to 25% 1-(((2S,3S,4S)-3-ethyl-4-fluoro-5-oxopyrrolidin-2-yl)methoxy)-7-methoxyisoquinoline-6-carboxamide.

A sustained release coating as known in the art, especially polymer coatings, may be employed to fabricate the membrane, as previously discussed for reservoir systems. Suitable and preferred polymer coating materials, equipment, and coating methods also include those previously discussed.

The rate of 1-(((2S,3S,4S)-3-ethyl-4-fluoro-5-oxopyrrolidin-2-yl)methoxy)-7-methoxyisoquinoline-6-carboxamide release from the coated multiparticulates can also be controlled by factors such as the composition and binder content of the drug-containing core, the thickness and permeability of the coating, and the surface-to-volume ratio of the multiparticulates. It will be appreciated by those skilled in the art that increasing the thickness of the coating will decrease the release rate, whereas increasing the permeability of the coating or the surface-to-volume ratio of the multiparticulates will increase the release rate. If desired, the permeability of the coating may be adjusted by blending of two or more materials. A useful series of coatings comprises mixtures of water-insoluble and water-soluble polymers, for example, ethylcellulose and hydroxypropyl methylcellulose, respectively. A useful modification to the coating is the addition of finely-divided water-soluble material, such as sugars or salts. When placed in an aqueous medium, these water soluble membrane additives are leached out of the membrane, leaving pores which facilitate delivery of the drug. The membrane coating may also be modified by the addition of plasticizers, as is known to those skilled in the art. Another useful variation of the membrane coating utilizes a mixture of solvents chosen such that as the coating dries, a phase inversion takes place in the applied coating solution, resulting in a membrane with a porous structure.

Another class of dosage forms includes those forms which incorporate a delay before the onset of controlled release of 1-(((2S,3S,4S)-3-ethyl-4-fluoro-5-oxopyrrolidin-2-yl)methoxy)-7-methoxyisoquinoline-6-carboxamide. One embodiment can be illustrated by a tablet comprising a core containing 1-(((2S,3S,4S)-3-ethyl-4-fluoro-5-oxopyrrolidin-2-yl)methoxy)-7-methoxyisoquinoline-6-carboxamide coated with a first coating of a polymeric material of the type useful for controlled release of 1-(((2S,3S,4S)-3-ethyl-4-fluoro-5-oxopyrrolidin-2-yl)methoxy)-7-methoxyisoquinoline-6-carboxamide and a second coating of the type useful for delaying release of drugs when the dosage form is ingested. The first coating is applied over and surrounds the tablet. The second coating is applied over and surrounds the first coating.

The tablet can be prepared by techniques well known in the art and contains a therapeutically useful amount of 1-(((2S,3S,4S)-3-ethyl-4-fluoro-5-oxopyrrolidin-2-yl)methoxy)-7-methoxyisoquinoline-6-carboxamide plus such excipients as are necessary to form the tablet by such techniques.

The first coating may be a controlled release coating as known in the art, especially polymer coatings, to fabricate the membrane, as previously discussed for reservoir systems. Suitable polymer coating materials, equipment, and coating methods also include those previously discussed.

Materials useful for preparing the second coating on the tablet include polymers known in the art as enteric coatings for delayed-release of pharmaceuticals. These most commonly are pH-sensitive materials such as cellulose acetate phthalate, cellulose acetate trimellitate, hydroxypropyl methyl cellulose phthalate, poly(vinyl acetate phthalate), and acrylic copolymers such as Eudragit L-100 (RohmPharma), Eudragit L 30 D-55, Eudragit S 100, Eudragit FS 30D, and related materials, as more fully detailed below under “Delayed Release”. The thickness and type of the delayed-release coating is adjusted to give the desired delay property. In general, thicker coatings are more resistant to erosion and, consequently, yield a longer delay as do coatings which are designed to dissolve above pH 7. Preferred coatings typically range from about 10 micron in thickness to about 3 mm in thickness and more preferably 10 um to 500 um.

When ingested, the twice-coated tablet passes through the stomach, where the second coating prevents release of the 1-(((2S,3S,4S)-3-ethyl-4-fluoro-5-oxopyrrolidin-2-yl)methoxy)-7-methoxyisoquinoline-6-carboxamide under the acidic conditions prevalent there. When the tablet passes out of the stomach and into the small intestine, where the pH is higher, the second coating erodes or dissolves according to the physicochemical properties of the chosen material. Upon erosion or dissolution of the second coating, the first coating prevents immediate or rapid release of the 1-(((2S,3S,4S)-3-ethyl-4-fluoro-5-oxopyrrolidin-2-yl)methoxy)-7-methoxyisoquinoline-6-carboxamide and modulates the release so as to prevent the production of high concentrations, thereby minimizing side-effects.

Another embodiment comprises a multiparticulate wherein each particle is dual coated as described above for tablets, first with a polymer designed to yield controlled release of the 1-(((2S,3S,4S)-3-ethyl-4-fluoro-5-oxopyrrolidin-2-yl)methoxy)-7-methoxyisoquinoline-6-carboxamide and then coated with a polymer designed to delay onset of release in the environment of the GI tract when the dosage form is ingested. The beads contain 1-(((2S,3S,4S)-3-ethyl-4-fluoro-5-oxopyrrolidin-2-yl)methoxy)-7-methoxyisoquinoline-6-carboxamide and may contain one or more excipients as needed for fabrication and performance. Multiparticulates which contain a high fraction of 1-(((2S,3S,4S)-3-ethyl-4-fluoro-5-oxopyrrolidin-2-yl)methoxy)-7-methoxyisoquinoline-6-carboxamide relative to binder are desired. The multiparticulate may be of a composition and be fabricated by any of the techniques previously disclosed for multiparticulates used to make reservoir systems (including extrusion and spheronization, wet granulation, fluid bed granulation, and rotary bed granulation, seed building, and so forth).

The controlled release coating may be as known in the art, especially polymer coatings, to fabricate the membrane, as previously discussed for reservoir systems. Suitable polymer coating materials, equipment, and coating methods also include those previously discussed.

The rate of 1-(((2S,3S,4S)-3-ethyl-4-fluoro-5-oxopyrrolidin-2-yl)methoxy)-7-methoxyisoquinoline-6-carboxamide release from the controlled-release-coated multiparticulates (e.g., the multiparticulates before they receive the delayed-release coating) and methods of modifying the coating are also controlled by the factors previously discussed for reservoir system 1-(((2S,3S,4S)-3-ethyl-4-fluoro-5-oxopyrrolidin-2-yl)methoxy)-7-methoxyisoquinoline-6-carboxamide multiparticulates.

The second membrane or coating for dual coated multiparticulates is a delayed-release coating which is applied over the first controlled-release coating, as disclosed above for tablets, and may be formed from the same materials. It should be noted that the use of the so-called “enteric” materials to practice this embodiment differs significantly from their use to produce conventional enteric dosage forms. With conventional enteric forms, the object is to delay release of the drug until the dosage form has passed the stomach and then to deliver the dose shortly after emptying from the stomach. Dosing of 1-(((2S,3S,4S)-3-ethyl-4-fluoro-5-oxopyrrolidin-2-yl)methoxy)-7-methoxyisoquinoline-6-carboxamide directly and completely to the duodenum is undesirable, however, due to local metabolism which is sought to be minimized or avoided by this invention. Therefore, if conventional enteric polymers are to be used to practice this embodiment, it may be necessary to apply them significantly more thickly than in conventional practice, in order to delay drug release until the dosage form reaches the lower GI tract. However, it is preferred to effect a controlled delivery of 1-(((2S,3S,4S)-3-ethyl-4-fluoro-5-oxopyrrolidin-2-yl)methoxy)-7-methoxyisoquinoline-6-carboxamide after the delayed-release coating has dissolved or eroded, therefore the benefits of this embodiment may be realized with a proper combination of delayed-release character with controlled-release character, and the delayed-release part alone may or may not necessarily conform to USP enteric criteria. The thickness of the delayed-release coating is adjusted to give the desired delay property. In general, thicker coatings are more resistant to erosion and, consequently, yield a longer delay.

It should also be noted, that sustained release osmotic systems as defined above, could also be defined in the current delay then controlled release category. Typical osmotic sustained release systems have an initial delay of 0.5-6 hours prior to drug release in a controlled fashion. In this manner, a standard osmotic monolithic or bilayer sustained release system embodies the definition of delay followed by controlled release.

In another embodiment (“bursting osmotic core device”), 1-(((2S,3S,4S)-3-ethyl-4-fluoro-5-oxopyrrolidin-2-yl)methoxy)-7-methoxyisoquinoline-6-carboxamide is incorporated in an osmotic bursting device which comprises a tablet core or bead core containing 1-(((2S,3S,4S)-3-ethyl-4-fluoro-5-oxopyrrolidin-2-yl)methoxy)-7-methoxyisoquinoline-6-carboxamide and, optionally, one or more osmogens. Devices of this type have been generally disclosed in Baker, U.S. Pat. No. 3,952,741, which is incorporated herein by reference. Examples of osmogens are sugars such as glucose, sucrose, mannitol, lactose, dextrates and the like; and salts such as sodium chloride, potassium chloride, sodium carbonate, and the like; water-soluble acids such as tartaric acid, fumaric acid, and the like. The 1-(((2S,3S,4S)-3-ethyl-4-fluoro-5-oxopyrrolidin-2-yl)methoxy)-7-methoxyisoquinoline-6-carboxamide-containing tablet core or bead core is coated with a polymer which forms a semipermeable membrane, that is, a membrane which is permeable to water but is substantially impermeable to 1-(((2S,3S,4S)-3-ethyl-4-fluoro-5-oxopyrrolidin-2-yl)methoxy)-7-methoxyisoquinoline-6-carboxamide. Examples of polymers which provide a semipermeable membrane are cellulose acetate, cellulose acetate butyrate, and ethylcellulose, preferably cellulose acetate. The semipermeable coating membrane may alternatively be composed of one or more waxes, such as insect and animal waxes such as beeswax, and vegetable waxes such as carnauba wax and hydrogenated vegetable oils. A melt mixture of a polyethylene glycol, e.g., polyethylene glycol-6000, and a hydrogenated oil, e.g., hydrogenated castor oil, may be used as a coating, as described for isoniazid tablets by Yoshino (Capsugel Symposia Series; Current Status on Targeted Drug Delivery to the Gastrointestinal Tract; 1993; pp. 185-190). Some preferred semipermeable coating materials are cellulose esters and cellulose ethers, polyacrylic acid derivatives such as polyacrylates and polyacrylate esters, and polyvinyl alcohols and polyalkenes such as ethylene vinyl alcohol copolymer. Other semipermeable coating materials are cellulose acetate and cellulose acetate butyrate.

When a coated tablet or bead of the “bursting osmotic core” embodiment of this invention is placed in an aqueous environment of use, water passes through the semipermeable membrane into the core, dissolving a portion of the 1-(((2S,3S,4S)-3-ethyl-4-fluoro-5-oxopyrrolidin-2-yl)methoxy)-7-methoxyisoquinoline-6-carboxamide and osmogen or osmogens, generating a colloidal osmotic pressure which results in bursting of the semipermeable membrane and release of 1-(((2S,3S,4S)-3-ethyl-4-fluoro-5-oxopyrrolidin-2-yl)methoxy)-7-methoxyisoquinoline-6-carboxamide into the aqueous environment. By choice of bead or tablet core size and geometry, identity and quantity of osmogen or osmogens, and thickness of the semipermeable membrane, the time lag between placement of the dosage form into the aqueous environment of use and release of the enclosed 1-(((2S,3S,4S)-3-ethyl-4-fluoro-5-oxopyrrolidin-2-yl)methoxy)-7-methoxyisoquinoline-6-carboxamide may be chosen. It will be appreciated by those skilled in the art that increasing the surface-to-volume ratio of the dosage form, and increasing the osmotic activity of the osmogen or osmogens serve to decrease the time lag, whereas increasing the thickness of the coating will increase the time lag. Osmotic-bursting devices of this invention are those which exhibit substantially no release of 1-(((2S,3S,4S)-3-ethyl-4-fluoro-5-oxopyrrolidin-2-yl)methoxy)-7-methoxyisoquinoline-6-carboxamide from the dosage form until the dosage form has exited the stomach and has resided in the small intestine for about 15 minutes or greater. Some osmotic-bursting devices exhibit substantially no release of 1-(((2S,3S,4S)-3-ethyl-4-fluoro-5-oxopyrrolidin-2-yl)methoxy)-7-methoxyisoquinoline-6-carboxamide from the dosage form until the dosage form has exited the stomach and has resided in the small intestine for about 30 minutes or greater. Other osmotic-bursting devices exhibit substantially no release of 1-(((2S,3S,4S)-3-ethyl-4-fluoro-5-oxopyrrolidin-2-yl)methoxy)-7-methoxyisoquinoline-6-carboxamide from the dosage form until the dosage form has exited the stomach and has resided in the small intestine for about 90 minutes or greater. Still other osmotic-bursting devices exhibit substantially no release of 1-(((2S,3S,4S)-3-ethyl-4-fluoro-5-oxopyrrolidin-2-yl)methoxy)-7-methoxyisoquinoline-6-carboxamide from the dosage form until the dosage form has exited the stomach and has resided in the small intestine for and most preferably 3 hours or greater, thus assuring that minimal 1-(((2S,3S,4S)-3-ethyl-4-fluoro-5-oxopyrrolidin-2-yl)methoxy)-7-methoxyisoquinoline-6-carboxamide is released in the duodenum and upper small intestine.

A bursting osmotic core device possesses no mechanism for “sensing” that the device has exited the stomach and entered the duodenum. Thus devices of this type release 1-(((2S,3S,4S)-3-ethyl-4-fluoro-5-oxopyrrolidin-2-yl)methoxy)-7-methoxyisoquinoline-6-carboxamide at a predetermined time after entering an aqueous environment, e.g., after being swallowed. In the fasted state, indigestible non-disintegrating solids, such as the “bursting osmotic core devices” of this invention, are emptied from the stomach during phase III of the Interdigestive Migrating Myoelectric Complex (IMMC), which occurs approximately every 2 hr in the human. Depending on the stage of the IMMC at the time of dosing in the fasted state, a bursting osmotic core device may exit the stomach almost immediately after dosing, or as long as 2 hr after dosing. In the fed state, indigestible non-disintegrating solids, which are <11 mm in diameter, will empty slowly from the stomach with the contents of the meal (Khosla and Davis, Int. J. Pharmaceut. 62 (1990) R9-R11). If the indigestible non-disintegrating solid is greater than about 11 mm in diameter, e.g., about the size of a typical tablet, it will be retained in the stomach for the duration of the digestion of the meal, and will exit into the duodenum during phase III of an IMMC, after the entire meal has been digested and has exited the stomach.

In a further embodiment, a “bursting coated swelling core”, a 1-(((2S,3S,4S)-3-ethyl-4-fluoro-5-oxopyrrolidin-2-yl)methoxy)-7-methoxyisoquinoline-6-carboxamide-containing tablet or bead is prepared which also comprises 25-70% of a swellable material, such as a swellable colloid (e.g., gelatin), as described in Milosovich, U.S. Pat. No. 3,247,066, incorporated herein by reference. Swelling core materials are hydrogels, e.g., hydrophilic polymers which take up water and swell, such as polyethylene oxides, polyacrylic acid derivatives such as polymethyl methacrylate, polyacrylamides, polyvinyl alcohol, poly-N-vinyl-2-pyrrolidone, carboxymethylcellulose, starches, and the like. Swelling hydrogels for this embodiment include polyethylene oxides, carboxymethylcellulose and croscarmellose sodium. The colloid/hydrogel-containing 1-(((2S,3S,4S)-3-ethyl-4-fluoro-5-oxopyrrolidin-2-yl)methoxy)-7-methoxyisoquinoline-6-carboxamide-containing core tablet or bead is coated, at least in part, by a semipermeable membrane. Examples of polymers which provide a semipermeable membrane are cellulose acetate and cellulose acetate butyrate, and ethylcellulose. The semipermeable coating membrane may alternatively be composed of one or more waxes, such as insect and animal waxes such as beeswax, and vegetable waxes such as carnauba wax and hydrogenated vegetable oils. A melt mixture of a polyethylene glycol, e.g., polyethylene glycol-6000, and a hydrogenated oil, e.g., hydrogenated castor oil, may be used as a coating, as described for isoniazid tablets by Yoshino (Capsugel Symposia Series; Current Status on Targeted Drug Delivery to the Gastrointestinal Tract; 1993; pp. 185-190). Some semipermeable coating materials are cellulose esters and cellulose ethers, polyacrylic acid derivatives such as polyacrylates and polyacrylate esters, polyvinyl alcohols and polyalkenes such as ethylene vinyl alcohol copolymer, cellulose acetate and cellulose acetate butyrate.

When a coated tablet or bead having a bursting coated swelling core is placed in an aqueous environment of use, water passes through the semipermeable membrane into the core, swelling the core and resulting in bursting of the semipermeable membrane and release of 1-(((2S,3S,4S)-3-ethyl-4-fluoro-5-oxopyrrolidin-2-yl)methoxy)-7-methoxyisoquinoline-6-carboxamide into the aqueous environment. By choice of bead or tablet core size and geometry, identity and quantity of swelling agent, and thickness of the semipermeable membrane, the time lag between placement of the dosage form into the aqueous environment of use and release of the enclosed 1-(((2S,3S,4S)-3-ethyl-4-fluoro-5-oxopyrrolidin-2-yl)methoxy)-7-methoxyisoquinoline-6-carboxamide may be chosen. Preferred bursting coated swelling core devices of this invention are those which exhibit substantially no release of 1-(((2S,3S,4S)-3-ethyl-4-fluoro-5-oxopyrrolidin-2-yl)methoxy)-7-methoxyisoquinoline-6-carboxamide from the dosage form until the dosage form has exited the stomach and has resided in the small intestine for about 15 minutes or greater, preferably about 30 minutes or greater, thus assuring that minimal 1-(((2S,3S,4S)-3-ethyl-4-fluoro-5-oxopyrrolidin-2-yl)methoxy)-7-methoxyisoquinoline-6-carboxamide is released in the duodenum.

A bursting coated swelling core device possesses no mechanism for sensing that the device has exited the stomach and entered the duodenum. Thus devices of this type release their 1-(((2S,3S,4S)-3-ethyl-4-fluoro-5-oxopyrrolidin-2-yl)methoxy)-7-methoxyisoquinoline-6-carboxamide contents at a predetermined time after entering an aqueous environment, e.g., after being swallowed, as previously discussed for bursting osmotic core devices, and the same consideration and preferences apply to making bursting coated swelling core devices. Bursting coated swelling core devices may be combined with immediate release devices to create a dosage form that will release drug both immediately after administration and at one or more additional predetermined times after dosing.

In a further embodiment, a “pH-triggered osmotic bursting device”, 1-(((2S,3S,4S)-3-ethyl-4-fluoro-5-oxopyrrolidin-2-yl)methoxy)-7-methoxyisoquinoline-6-carboxamide is incorporated into a device of the type described in allowed commonly assigned co-pending U.S. Pat. No. 5,358,502, issued Oct. 25, 1994, incorporated herein by reference. The device comprises 1-(((2S,3S,4S)-3-ethyl-4-fluoro-5-oxopyrrolidin-2-yl)methoxy)-7-methoxyisoquinoline-6-carboxamide and optionally one or more osmogens, surrounded at least in part by a semipermeable membrane. The semipermeable membrane is permeable to water and substantially impermeable to 1-(((2S,3S,4S)-3-ethyl-4-fluoro-5-oxopyrrolidin-2-yl)methoxy)-7-methoxyisoquinoline-6-carboxamide and osmogen. Useful osmogens are the same as those described above for bursting osmotic core devices. Useful semipermeable membrane materials are the same as those described above for bursting osmotic core devices. A pH-trigger means is attached to the semipermeable membrane. The pH-trigger means is activated by a pH above 5.0, and triggers the sudden delivery of the 1-(((2S,3S,4S)-3-ethyl-4-fluoro-5-oxopyrrolidin-2-yl)methoxy)-7-methoxyisoquinoline-6-carboxamide. In this embodiment, the pH-trigger means comprises a membrane or polymer coating which surrounds the semipermeable coating. The pH-trigger coating contains a polymer which is substantially impermeable and insoluble in the pH range of the stomach, but becomes permeable and soluble at about the pH of the duodenum, about pH 6.0.

Exemplary pH-sensitive polymers are polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH-sensitive polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers. As described above cellulose acetate phthalate is available as a latex under the tradename Aquateric® (registered trademark of FMC Corp., Philadelphia, Pa.), and acrylic copolymers are available under the tradenames Eudragit-R® and Eudragit-L®. For appropriate application in this embodiment, these polymers should be plasticized utilizing plasticizers described above. The pH-trigger coating may also comprise a mixture of polymers, for example cellulose acetate and cellulose acetate phthalate. Another suitable mixture comprises Eudragit-L® and Eudragit-S®; the ratio of the two, and the coating thickness, defining the sensitivity of the “trigger”, e.g., the pH at which the outer pH-trigger coating weakens or dissolves.

A pH-triggered osmotic bursting device generally operates as follows. After oral ingestion, the pH-trigger coating, which surrounds the semipermeable coating, which in turn surrounds the 1-(((2S,3S,4S)-3-ethyl-4-fluoro-5-oxopyrrolidin-2-yl)methoxy)-7-methoxyisoquinoline-6-carboxamide-containing core tablet or bead, remains undissolved and intact in the stomach. In the stomach, water may or may not commence penetration through the pH-trigger coating and the semipermeable coating, thus starting hydration of the core, which contains 1-(((2S,3S,4S)-3-ethyl-4-fluoro-5-oxopyrrolidin-2-yl)methoxy)-7-methoxyisoquinoline-6-carboxamide and optional osmogen or osmogens. After the device has exited the stomach and has entered the small intestine, the pH-trigger coating rapidly disintegrates and dissolves, and water passes through the semipermeable coating, dissolving 1-(((2S,3S,4S)-3-ethyl-4-fluoro-5-oxopyrrolidin-2-yl)methoxy)-7-methoxyisoquinoline-6-carboxamide and optional osmogen within the core. As the colloidal osmotic pressure across the semipermeable coating exceeds some threshold value, the semipermeable coating fails, and the device bursts, releasing 1-(((2S,3S,4S)-3-ethyl-4-fluoro-5-oxopyrrolidin-2-yl)methoxy)-7-methoxyisoquinoline-6-carboxamide. It is preferred that this bursting and release of 1-(((2S,3S,4S)-3-ethyl-4-fluoro-5-oxopyrrolidin-2-yl)methoxy)-7-methoxyisoquinoline-6-carboxamide occur at about 15 minutes or more, preferably 30 minutes or more, after the pH-triggered osmotic bursting device exits the stomach and enters the duodenum, thus minimizing exposure of the sensitive duodenum to 1-(((2S,3S,4S)-3-ethyl-4-fluoro-5-oxopyrrolidin-2-yl)methoxy)-7-methoxyisoquinoline-6-carboxamide.

For a pH-triggered osmotic bursting device, the lag-time or delay-time is controlled by the choice and amount of osmogen or osmogens in the core, by the choice of semipermeable coating, and by the thickness of the semipermeable coating. It will be appreciated by those skilled in the art, for example, that a thicker semipermeable coating will result in a longer delay after the device has exited the stomach.

Advantageously, because a pH-triggered osmotic bursting device possesses a mechanism for sensing that the device has exited the stomach, intersubject variability in gastric emptying is not significant.

In a further embodiment, a “pH-triggered bursting coated swelling core”, a tablet core or bead containing 1-(((2S,3S,4S)-3-ethyl-4-fluoro-5-oxopyrrolidin-2-yl)methoxy)-7-methoxyisoquinoline-6-carboxamide and a swelling material is coated with a semipermeable coating which is further coated with a pH-sensitive coating. The core composition, including choice of swelling material is as described above for the bursting coated swelling core embodiment. The choice of semipermeable coating material and pH-sensitive coating material are as described above for the “pH-triggered osmotic core” embodiment. This device is described in detail in commonly-assigned copending U.S. patent application Ser. No. 08/023,227, filed Feb. 25, 1993, incorporated herein by reference.

A pH-triggered bursting swelling core embodiment generally operates as follows. After oral ingestion, the pH-trigger coating, which surrounds the semi-permeable coating, which in turn surrounds the 1-(((2S,3S,4S)-3-ethyl-4-fluoro-5-oxopyrrolidin-2-yl)methoxy)-7-methoxyisoquinoline-6-carboxamide-containing core tablet or bead, remains undissolved and intact in the stomach. In the stomach, water may or may not commence penetration through the pH-trigger coating and the semipermeable coating, thus starting hydration of the core, which contains 1-(((2S,3S,4S)-3-ethyl-4-fluoro-5-oxopyrrolidin-2-yl)methoxy)-7-methoxyisoquinoline-6-carboxamide and water-swellable material, preferably a hydrogel. When the pH-triggered bursting swelling core device exits the stomach and enters the small intestine, the pH-trigger coating rapidly disintegrates and dissolves, and water passes through the semipermeable coating, dissolving 1-(((2S,3S,4S)-3-ethyl-4-fluoro-5-oxopyrrolidin-2-yl)methoxy)-7-methoxyisoquinoline-6-carboxamide and swelling the water-swellable material within the core.

As the swelling pressure across the semipermeable coating exceeds some threshold value, the semipermeable coating fails, and the device bursts, releasing 1-(((2S,3S,4S)-3-ethyl-4-fluoro-5-oxopyrrolidin-2-yl)methoxy)-7-methoxyisoquinoline-6-carboxamide. This bursting and release of 1-(((2S,3S,4S)-3-ethyl-4-fluoro-5-oxopyrrolidin-2-yl)methoxy)-7-methoxyisoquinoline-6-carboxamide occurs at about 15 minutes or more, around about 30 minutes, after the pH-triggered bursting swelling core device exits the stomach and enters the duodenum, thus minimizing exposure of the sensitive duodenum to 1-(((2S,3S,4S)-3-ethyl-4-fluoro-5-oxopyrrolidin-2-yl)methoxy)-7-methoxyisoquinoline-6-carboxamide.

For the “pH-triggered bursting swelling core” device, the lag-time or delay-time can be controlled by the choice and amount of swelling material in the core, by the choice of semipermeable coating, and by the thickness of the semipermeable coating. It will be appreciated by those skilled in the art, for example, that a thicker semipermeable coating will result in a longer delay after the device has exited the stomach. A pH-triggered bursting swelling core device contains a bead or tablet core of 1-(((2S,3S,4S)-3-ethyl-4-fluoro-5-oxopyrrolidin-2-yl)methoxy)-7-methoxyisoquinoline-6-carboxamide with synthetic hydrogel, preferably carboxymethylcellulose.

Advantageously, because a pH-triggered bursting swelling core device possesses a mechanism for sensing that the device has exited the stomach, intersubject variability in gastric emptying is not significant. pH-triggered bursting swelling core devices may be combined with immediate release devices to create a dosage form that will release drug both immediately after administration and at one or more additional predetermined locations in the GI tract after dosing.

The formulation of 1-(((2S,3S,4S)-3-ethyl-4-fluoro-5-oxopyrrolidin-2-yl)methoxy)-7-methoxyisoquinoline-6-carboxamide used in the Phase 2 study to evaluate the safety and efficacy of PF-06650833 in subjects with RA and an inadequate response to methotrexate were SCT modified release tablets with unit dosage strengths of 20 mgs and 100 mgs (20 mg MR-FORM1 and 100 mg MR-FORM1, respectively). Because the SCT bilayer system was not capable of handling dosages greater than 100 mgs, multiple tablets were administered to subjects to achieve higher dosages. In order to eliminate administration of multiple tablets to subjects to achieve higher doses of PF-06650833, four new ECS single layer MR tablets were developed (100 mg MR-FORM2, 200 mg MR-FORM2, 100 mg MR-FORM3, and 200 mg MR-FORM3) for potential use in future clinical studies at 100 mg and 200 mg unit dosage strengths. The 100 mg MR-FORM2 and 200 mg MR-FORM2 formulations contain sorbitol and NaCl as osmogens and the 100 mg MR-FORM3 and 200 mg MR-FORM3 formulations contain dextrates and NaCl as the osmogens.

Table A provides the SCT bilayer MR formulation used to prepare ten thousand (batch) 100 mg tablets (100 mg MR-FORM1) used in the previous Phase 2 clinical study to evaluate PF-06650833 for treating RA and in the Phase 1 clinical study to evaluate the relative bioavailability and food effect of the MR-FORM1 formulation in healthy subjects (Table 3).

TABLE A
SCT BiLayer 100 mg MR Tablet Formulation
(100 mg MR-FORM1)

| Ingredient | Function | Amount (grams) |
|---|---|---|
| PF-06650833 | Active Ingredient | 1000.00^{a} |
| Polyethylene oxide (200,00 mol. wt.) | Entraining polymer | 3950.00^{b} |
| Magnesium stearate | Lubricant | 50.00 |
| Total Active Layer Batch Weight | | 5000.00 |
| Polyethylene oxide (5,000,00 mol. wt.) | Swelling Agent | 1382.10 |
| Sodium Chloride | Osmogen | 765.00 |
| Microcrystalline cellulose | Tableting aid | 382.50 |
| Magnesium Stearate | Lubricant | 12.75 |
| FD&C Blue Aluminum Lake #2 | Colorant | 7.65 |
| Total Sweller Layer Batch Weight | | 2550.00 |
| Total Bilayer Core Tablet Weight | | 7550.00 |
| Cellulose Acetate | Osmotic Membrane | 390.00 |
| Polyethylene Glycol | Plasticizer | 110.000 |
| Acetone | Processing aid | As Required^{c} |
| Purified Water | Processing aid | As Required^{c} |

| | |
|---|---|
| Total Osmotic Coating Batch Weight | 500.00 |
| Total Tablet Batch Weight | 8050.000 |

^{a}Based on a theoretical potency of 100%. The amount of PF-06650833 per tablet may be adjusted based on the measured potency.
^{b}Weight based on measured potency of PF-06650833 to maintain constant tablet weight.
^{c}Solvents are removed during processing. Amount as required to achieve desired coating attributes.

Table B provides the ECS single layer MR formulation, containing the osmogens sorbitol and NaCl, used to prepare the 100 mg tablets (100 mg MR-FORM2) used in the Phase 1 clinical study to evaluate the relative bioavailability and food effect of the MR-FORM2 formulation in healthy subjects (Table 3).

TABLE B
ECS Single Layer 100 mg MR Tablet Formulation
Containing Sorbitol and NaCl as Osmogens
(100 mg MR-FORM2)

| Ingredient | Function | Amount (mgs) |
|---|---|---|
| PF-06650833 | Active Ingredient | 100.000^{a} |
| Sorbitol | Osmogen | 316.000^{b} |
| Sodium Chloride | Osmogen | 240.000 |
| Hydroxyethylcellulose | Suspending Agent | 64.000 |
| Copovidone | Tableting Aid | 72.000 |
| Magnesium Stearate | Lubricant | 4.000 |
| Sodium Stearyl Fumarate | Lubricant | 4.000 |
| Total Active Core Weight | | 800.000 |
| Cellulose Acetate | Osmotic Membrane | 44.100 |
| Polyethylene Glycol | Plasticizer | 18.900 |
| Acetone | Processing aid | As Required⁰ |
| Purified Water | Processing aid | As Required⁰ |

| | |
|---|---|
| Total Osmotic Coating Weight | 63.000 |
| Total Tablet Weight | 863.000 |

^{a}Based on a theoretical potency of 100%. The amount of PF-06650833 per tablet may be adjusted based on the measured potency.
^{b}Weight based on measured potency of PF-06650833 to maintain constant tablet weight.
^{c}Solvents are removed during processing. Amount as required to achieve desired coating attributes.

Table B1 provides stability data for the ECS single layer 100 mg MR tablets containing sorbitol and NaCl as osmogens (100 mg MR-FORM2).

TABLE B1
Stability Data for the ECS Single Layer 100 mg MR Tablets
Containing Sorbitol and NaCl as Osmogens
(100 mg MR-FORM2)

| | | Total | |
|---|---|---|---|
| | | Degradation | |
| Storage | | Products | |
| Condition | Time | (%) | Appearance |
| −20° C., HDPE¹ Bottle | Initial | NMT² 0.05 | Off-White |
| 23 Tablets | 6 weeks | NMT0.05 | Off-White |
| 25° C./60% RH³ | Initial | NMT 0.05 | Off-White |
| HDPE Bottle | 6 weeks | NMT 0.05 | Off-White |
| 23 Tablets | 3 months | NMT 0.05 | Off-White |
| | 6 months | NMT 0.05 | Off-White |
| | 9 months | NMT 0.05 | Off-White |
| | 12 months | NMT 0.05 | Off-White |
| | 18 months | NMT 0.05 | Off-White |
| | 24 months | NMT 0.05 | Off-White |
| 30° C./75% RH | Initial | NMT 0.05 | Off-White |
| HDPE Bottle | 6 months | NMT 0.05 | Off-White |
| 23 Tablets | 12 months | NMT 0.05 | Off-White |
| | 24 months | NMT 0.05 | Off-White |
| 40° C./75% RH | Initial | NMT 0.05 | Off-White |
| HDPE Bottle | 6 weeks | NMT 0.05 | Off-White |
| 23 Tablets | 3 months | NMT 0.05 | Off-White |
| | 6 months | NMT 0.05 | Off-White |
| UV-FL⁴, HDPE Bottle | Initial | NMT 0.05 | Off-White |
| 23 Tablets | 8 days | NMT 0.05 | Off-White |
| UV-FL, Open Petri Dish | Initial | — | Off-White |
| 23 Tablets | 8 days | — | Light-yellow |

¹HDPE means High density polyethylene.
²NMT means “not more than.”
³RH means relative humidity.
⁴ICH photostability using ultraviolet and fluorescence lamps.

Table B2 provides dissolution data for the ECS single layer 100 mg MR tablets containing sorbitol and NaCl as osmogens (100 mg MR-FORM2).

TABLE B2
Dissolution Data for the ECS Single Layer 100 mg MR Tablets
Containing Sorbitol and NaCl as Osmogens
(100 mg MR-FORM2)

| | | Mean % Dissolved | | |
|---|---|---|---|---|
| Storage | | 2 | 6 | 12 |
| Condition | Time | hours | hours | hours |
| −20° C., HDPE¹ Bottle | Initial | 15 | 64 | 92 |
| 23 Tablets | 6 weeks | 15 | 63 | 91 |
| 25° C./60% RH² | Initial | 15 | 64 | 92 |
| HDPE Bottle | 6 weeks | 14 | 62 | 92 |
| 23 Tablets | 3 months | 14 | 62 | 91 |
| | 6 months | 14 | 62 | 91 |
| | 9 months | 14 | 62 | 91 |
| | 12 months | 14 | 61 | 90 |
| | 18 months | 15 | 63 | 91 |
| | 24 months | 14 | 63 | 90 |
| 30° C./75% RH | Initial | 15 | 64 | 92 |
| HDPE Bottle | 6 months | 13 | 58 | 89 |
| 23 Tablets | 12 months | 12 | 56 | 88 |
| | 24 months | 11 | 56 | 83 |
| 40° C./75% RH | Initial | 15 | 64 | 92 |
| HDPE Bottle | 6 weeks | 12 | 57 | 88 |
| 23 Tablets | 3 months | 11 | 53 | 86 |
| | 6 months | 11 | 52 | 85 |

¹HDPE means High density polyethylene.
²RH means relative humidity.

Table C provides the ECS single layer MR formulation, containing the osmogens sorbitol and NaCl, used to prepare the 200 mg tablets (200 mg MR-FORM2) used in the Phase 1 clinical study to evaluate the relative bioavailability and food effect of the MR-FORM2 formulation in healthy subjects (Table 3).

TABLE C
ECS Single Layer 200 mg MR Tablet Formulation
Containing Sorbitol and NaCl as Osmogens
(200 mg MR-FORM2)

| Ingredient | Function | Amount (mgs) |
|---|---|---|
| PF-06650833 | Active Ingredient | 200.000^{a} |
| Sorbitol | Osmogen | 216.000^{b} |
| Sodium Chloride | Osmogen | 240.000 |
| Hydroxyethylcellulose | Suspending Agent | 64.000 |
| Copovidone | Tableting Aid | 72.000 |
| Magnesium Stearate | Lubricant | 4.000 |
| Sodium Stearyl Fumarate | Lubricant | 4.000 |
| Total Active Core Weight | | 800.000 |
| Cellulose Acetate | Osmotic Membrane | 44.100 |
| Polyethylene Glycol | Plasticizer | 18.900 |
| Acetone | Processing aid | As Required^{c} |
| Purified Water | Processing aid | As Required^{c} |

| | |
|---|---|
| Total Osmotic Coating Weight | 63.000 |
| Total Tablet Weight | 863.000 |

^{a}Based on a theoretical potency of 100%. The amount of PF-06650833 per tablet may be adjusted based on the measured potency.
^{b}Weight based on measured potency of PF-06650833 to maintain constant tablet weight.
^{c}Solvents are removed during processing. Amount as required to achieve desired coating attributes.

Table C1 provides stability data for the ECS single layer 200 mg MR tablets containing sorbitol and NaCl as osmogens (200 mg MR-FORM2).

TABLE C1
Stability Data for the ECS Single Layer 200 mg MR Tablets
Containing Sorbitol and NaCl as Osmogens
(200 mg MR-FORM2)

| | | Total | |
|---|---|---|---|
| | | Degradation | |
| Storage | | Products | |
| Condition | Time | (%) | Appearance |
| −20° C., HDPE¹ Bottle | Initial | NMT² 0.05 | Off-White |
| 23 Tablets | 6 weeks | NMT0.05 | Off-White |
| 25° C./60% RH³ | Initial | NMT 0.07 | Off-White |
| HDPE Bottle | 6 weeks | NMT 0.05 | Off-White |
| 23 Tablets | 3 months | NMT 0.05 | Off-White |
| | 6 months | NMT 0.05 | Off-White |
| | 9 months | NMT 0.05 | Off-White |
| | 12 months | NMT 0.05 | Off-White |
| | 18 months | NMT 0.05 | Off-White |
| | 24 months | NMT 0.05 | Off-White |
| 30° C./75% RH | Initial | NMT 0.05 | Off-White |
| HDPE Bottle | 6 months | NMT 0.05 | Off-White |
| 23 Tablets | 12 months | NMT 0.05 | Off-White |
| | 24 months | NMT 0.05 | Off-White |
| 40° C./75% RH | Initial | NMT 0.05 | Off-White |
| HDPE Bottle | 6 weeks | NMT 0.05 | Off-White |
| 23 Tablets | 3 months | NMT 0.05 | Off-White |
| | 6 months | NMT 0.05 | Off-White |
| UV-FL⁴, HDPE Bottle | Initial | NMT 0.05 | Off-White |
| 23 Tablets | 8 days | NMT 0.05 | Off-White |
| UV-FL, Open Petri Dish | Initial | — | Off-White |
| 23 Tablets | 8 days | — | Light-yellow |

¹HDPE means High density polyethylene.
²NMT means “not more than.”
³RH means relative humidity.
⁴ICH photostability using ultraviolet and fluorescence lamps

Table C2 provides dissolution data for the ECS single layer 200 mg MR tablets containing sorbitol and NaCl as osmogens (200 mg MR-FORM2).

TABLE C2
Dissolution Data for the ECS Single Layer 200 mg MR Tablets
Containing Sorbitol and NaCl as Osmogens
(200 mg MR-FORM2)

| | | Mean % Dissolved | | |
|---|---|---|---|---|
| Storage | | 2 | 6 | 12 |
| Condition | Time | hours | hours | hours |
| −20° C., HDPE¹ Bottle | Initial | 19 | 65 | 91 |
| 23 Tablets | 6 weeks | 18 | 65 | 91 |
| 25° C./60% RH² | Initial | 19 | 65 | 91 |
| HDPE Bottle | 6 weeks | 18 | 65 | 91 |
| 23 Tablets | 3 months | 17 | 63 | 91 |
| | 6 months | 18 | 65 | 91 |
| | 9 months | 17 | 65 | 91 |
| | 12 months | 16 | 61 | 89 |
| | 18 months | 17 | 64 | 91 |
| | 24 months | 15 | 60 | 89 |
| 30° C./75% RH | Initial | 19 | 65 | 91 |
| HDPE Bottle | 6 months | 15 | 59 | 87 |
| 23 Tablets | 12 months | 15 | 60 | 89 |
| | 24 months | 12 | 55 | 86 |
| 40° C./75% RH | Initial | 19 | 65 | 91 |
| HDPE Bottle | 6 weeks | 17 | 63 | 91 |
| 23 Tablets | 3 months | 13 | 57 | 89 |
| | 6 months | 12 | 53 | 85 |

¹HDPE means High density polyethylene.
²RH means relative humidity.

TABLE D
ECS Single Layer 100 mg MR Tablet Formulation
Containing Dextrates and NaCl as Osmogens
(100 mg MR-FORM3)

| Ingredient | Function | Amount (mgs) |
|---|---|---|
| PF-06650833 | Active Ingredient | 100.000^{a} |
| Dextrates | Osmogen | 392.480^{b} |
| Sodium Chloride | Osmogen | 243.270 |
| Hydroxyethylcellulose | Suspending Agent | 72.000 |
| Colloidal Silicon Dioxide | Glidant | 2.250 |
| Copovidone | Tableting Aid | 81.000 |
| Magnesium Stearate | Lubricant | 4.500 |
| Sodium Stearyl Fumarate | Lubricant | 4.500 |
| Total Active Core Weight | | 900.000 |
| Cellulose Acetate | Osmotic Membrane | 28.860 |
| Polyethylene Glycol | Plasticizer | 8.140 |
| Acetone | Processing aid | As Required^{c} |
| Purified Water | Processing aid | As Required^{c} |

| | |
|---|---|
| Total Osmotic Coating Weight | 37.000 |
| Total Tablet Weight | 937.000 |

^{a}Based on a theoretical potency of 100%. The amount of PF-06650833 per tablet may be adjusted based on the measured potency.
^{b}Weight based on measured potency of PF-06650833 to maintain constant tablet weight.
^{c}Solvents are removed during processing. Amount as required to achieve desired coating attributes.

Table 1 provides the ECS single layer MR formulation, containing the osmogens dextrates and NaCl, used to prepare the 200 mg tablets (200 mg MR-FORM3) for use in: the Phase 2 clinical study to evaluate PF-06650833 for treating hidradenitis suppurativa; the Phase 2 clinical study to evaluate the combination therapy PF-06650833 and PF-06651600 for treating RA; and the Phase 2 clinical study to evaluate the combination therapy PF-06650833 and tofacitinib for treating RA.

TABLE 1
ECS Single Layer 200 mg MR Tablet Formulation
Containing Dextrates and NaCl as Osmogens
(200 mg MR-FORM3)

| Ingredient | Function | Amount (mgs) |
|---|---|---|
| PF-06650833 | Active Ingredient | 200.000^{a} |
| Dextrates | Osmogen | 330.750^{b} |
| Sodium Chloride | Osmogen | 205.000 |
| Hydroxyethylcellulose | Suspending Agent | 72.000 |
| Colloidal Silicon Dioxide | Glidant | 2.250 |
| Copovidone | Tableting Aid | 81.000 |
| Magnesium Stearate | Lubricant | 4.500 |
| Sodium Stearyl Fumarate | Lubricant | 4.500 |
| Total Active Core Weight | | 900.000 |
| Cellulose Acetate | Osmotic Membrane | 28.860 |
| Polyethylene Glycol | Plasticizer | 8.140 |
| Acetone | Processing aid | As Required^{c} |
| Purified Water | Processing aid | As Required^{c} |

| | |
|---|---|
| Total Osmotic Coating Weight | 37.000 |
| Total Tablet Weight | 937.000 |

^{a}Based on a theoretical potency of 100%. The amount of PF-06650833 per tablet may be adjusted based on the measured potency.
^{b}Weight based on measured potency of PF-06650833 to maintain constant tablet weight.
^{c}Solvents are removed during processing. Amount as required to achieve desired coating attributes.

Table 2 provides stability data for the ECS single layer 200 mg MR tablets containing dextrates and NaCl as osmogens (200 mg MR-FORM3).

TABLE 2
Stability Data for the ECS Single Layer 200 mg MR Tablets
Containing Dextrates and NaCl as Osmogens
(200 mg MR-FORM3)

| | | Total | |
|---|---|---|---|
| | | Degradation | |
| Storage | | Products | |
| Condition | Time | (%) | Appearance |
| 5° C. | Initial | — | — |
| HDPE¹ Bottle | 3 months | NMT² 0.05 | Off-White |
| 20 Tablets | 28 weeks | NMT 0.05 | Off-White |
| | 13 months | NMT 0.05 | Off-White |
| 25° C./60% RH³ | Initial | — | — |
| HDPE⁴ Bottle | 3 months | NMT 0.05 | Off-White |
| 20 Tablets | 6 months | NMT 0.05 | Off-White |
| | 13 months | NMT 0.05 | Off-White |
| 40° C./75% RH | Initial | — | — |
| HDPE Bottle | 3 months | NMT 0.05 | Off-White |
| 20 Tablets | 6 months | NMT 0.05 | Off-White |

¹HDPE means High density polyethylene.
²NMT means “not more than.”
³RH means relative humidity.
⁴ICH photostability using ultraviolet and fluorescence lamps.

Table 2A provides dissolution data for the ECS single layer 200 mg MR tablets containing dextrates and NaCl as osmogens (200 mg MR-FORM3).

TABLE 2A
Dissolution Data for the ECS Single Layer 200 mg MR Tablets
Containing Dextrates and NaCl as Osmogens
(200 mg MR-FORM3)

| | | Mean % Dissolved | | |
|---|---|---|---|---|
| Storage | | 2 | 6 | 12 |
| Condition | Time | hours | hours | hours |
| 5° C. | Initial | — | — | — |
| HDPE¹ Bottle | 3 months | 19 | 66 | 92 |
| 20 Tablets | 28 weeks | 19 | 65 | 91 |
| | 13 months | 19 | 67 | 93 |
| 25° C./60% RH² | Initial | — | — | — |
| HDPE Bottle | 3 months | 18 | 63 | 91 |
| 20 Tablets | 6 months | 19 | 65 | 91 |
| | 13 months | 19 | 65 | 92 |
| 40° C./75% RH | Initial | — | — | — |
| HDPE Bottle | 3 months | 17 | 60 | 89 |
| 20 Tablets | 6 months | 16 | 58 | 87 |

¹HDPE means High density polyethylene.

Table 2B provides comparative dissolution data for the: ECS single layer 100 mg MR tablets containing sorbitol and NaCl as osmogens (100 mg MR-FORM2); ECS single layer 200 mg MR tablets containing sorbitol and NaCl as osmogens (200 mg MR-FORM2); and ECS single layer 200 mg MR tablets containing dextrates and NaCl as osmogens (200 mg MR-FORM3). The dissolution data was generated using an USP apparatus II (paddles) at 100 rpm in 1000±10 mLs of medium composed of 50 mM sodium phosphate buffer (pH 6.8) with 0.25% sodium dodecyl sulfate in purified water (18.20 grade) at 37° C.±5° C. Dissolved PF-06650833 was detected by UV spectrophotometry (353 nm) and compared against a standard solution.

TABLE 2B
Comparative Dissolution Data for the (100 mg MR-FORM2),
(200 mg MR-FORM2), and (200 mg MR-FORM3) Tablets

| | Mean % Dissolved | | | | | | |
|---|---|---|---|---|---|---|---|
| | 2 | 4 | 6 | 8 | 10 | 12 | 16 |
| | hours | hours | hours | hours | hours | hours | hours |
| 100 mg MR-FORM2 | 15 | — | 64 | — | — | 92 | — |
| 200 mg MR-FORM2 | 19 | — | 65 | — | — | 91 | — |
| 200 mg MR-FORM3 | 19 | 44 | 63 | 75 | 83 | 88 | 93 |

Residual solvents or processing aids used to prepare the 100 mg MR-FORM2, 200 mg MR-FORM2, 100 mg MR-FORM3, and 200 mg FORM3 tablets include acetone. The method used to determine the absence of acetone in the MR tablets of Tables B, C, D, and 1 utilized: a J&W Scientific DB-624 30 m×0.32 mm 1.8 μm or equivalent; helium as the carrier gas at a flow rate of 1.6 mL/min; an injection temperature of 180° C. split ratio 30:1; at a column/oven temperature of 40° C. for 6 minutes, increased at 30° C./minute to 225° C. and held at 225° C. for 4 minutes (typical run time 16.17 minutes).

The method used for the determination of degradation products of PF-06650833 MR tablets in Tables B1, C1, and 2 was reversed-phase ultra performance liquid chromatography (UPLC). The UPLC conditions used included: an ACE Excel 2 C4 2.1×150 mm 2 μm column; a mobile phase of 0.1% perchloric acid in acetonitrile; 46 minutes run time; and an UV absorbance detector at 210 nm. Quantification of degradation products was achieved by area percent. The new MR tablets, 100 mg MR-FORM2, 200 mg MR-FORM2, and 200 mg FORM3, were found to have not-more-than (NMT) 0.05% total degradants under a variety of durations, temperatures, and % RH described in Tables B1, C1, and 2.

The property most relevant to drug bioperformance is the dissolution rate. The target dissolution release profile for PF-06650833 is to release 80%±10%, preferably 80%±5%, of PF-06650833 at the eighth (8^{th}) hour in a controlled or modified manner. In addition, the dosage form should have no dose dumping or significantly incomplete release at the end of dissolution testing (e.g. 16^{th} hour). The comparative dissolution data provided in Table 2B demonstrates that the 100 mg MR-FORM2, 200 mg MR-FORM2, and 200 mg MR-FORM3 tablets all have similar and acceptable dissolution profiles of approximately 80% dissolution after eight (8) hours.

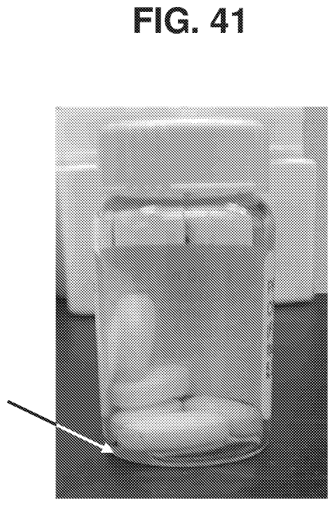

While the 100 mg MR-FORM2 and 200 mg MR-FORM2 tablets deliver the desired dissolution release profiles, they require storage with protection from humidity (NMT-45% RH) to have adequate physical stability. When exposed to humidity conditions higher than ˜45% RH, the sorbitol containing formulations, 100 mg MR-FORM2 and 200 mg MR-FORM2, undergo deliquescence. The absorption of water from the atmosphere results in dissolution of PF-06650833 within the tablets followed by leaking out of the delivery port as shown in FIG. 41. By changing the osmogent system from sorbitol and NaCl to dextrates and NaCl, the physical stability was improved in the 200 mg MR-FORM3 tablets under ambient temperature at ˜63% RH.

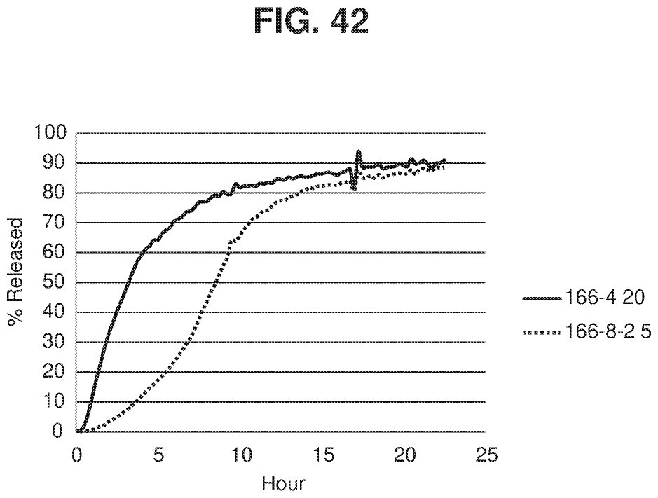

With regard to coating, the ECS tablets require a semi-permeable membrane that is stable during dissolution to achieve the desired in vitro and in vivo dissolution profiles. Membranes with unsuitable compositions may burst during dissolution and fail to achieve the desired dissolution profile. For example, the 200 mg MR-FORM2 tablets were coated with 60% cellulose acetate (CA) and 40% hydroxylpropylcellulose (HPC) that burst on a majority of the tablets during dissolution testing resulting in erratic dissolution profiles. FIG. 42 demonstrates the effect on the dissolution rate for tablets with the 60% CA/40% HPC coatings that burst during dissolution. Lot 166-4 20 released too much PF-06650833 within the first three hours due to bursting of the coating while lot 166-8-2 5 released PF-06650833 too slow upon bursting of the coat. The overall effect of an unstable coating is an unpredictable/erratic dissolution profile. It was discovered that a coating composed of 78% cellulose acetate (CA) and 22% polyethylene glycol (PEG) provided a stable/non-bursting coating resulting in an acceptable modified release of PF-06650833 achieving ˜80% dissolution after 8 hours for the 200 mg MR-FORM2 tablets.

A comparative pharmacokinetic (pK) evaluation, Table 3, was conducted for the 100 mg MR-FORM1 tablets, the 100 mg MR-Form 2 tablets, and the 200 mg MR-FORM2 tablets administered orally once daily in 24 healthy males age 24 to 51 with a mean body weight and body mass index (BMI) of 81.5 kgs and 25.6 kgs/m², respectively. The results in Table 3 demonstrate that the 100 mg MR-FORM2 and 200 mg MR-Form2 tablets have similar pK profiles to the 100 mg MR-FORM1 tablets administered fasted or fed in healthy male subjects.

TABLE 2C
Comparative Particle Size of PF-06650833 for Jetmilled, Fitzmilled, and Unmilled Processing

| Milling | D[v, 0.1] | D[v, 0.5] | D[v, 0.9] | D[4, 3] |
|---|---|---|---|---|
| Process | (μm) | (μm) | (μm) | (μm) |
| Jetmilled | 0.7 | 3.9 | 9.6 | 4.6 |
| Fitzmilled | 2.0 | 10.0 | 35.0 | 17.0 |
| Unmilled | 4.6 | 22.3 | 61.6 | 28.4 |

Jetmilled PF-06650833 particles were used in manufacturing the SCT bilayer tablets (20 mg and 100 mg MR-FORM1 tablets). For the ECS tablets containing sorbitol and NaCl as osmogens, fitzmilled PF-06650833 particles were used to prepare the 100 mg and 200 mg single layer ECS tablets (100 mg MR-FORM2 and 200 mg MR-Form2 tablets). The jetmilled and fitzmilled PF-06650833 particles required a dry granulation process to be used to compensate for the poor flow properties exhibited by the formulated blend.

It was discovered that unmilled PF-06650833 where particle size was controlled directly via crystallization improved manufacturing properties, in particular flow rate. The larger particle size enabled the use of direct compression for manufacture of the 100 mg and 200 mg ECS tablets containing dextrates and NaCl as osmogens (100 mg and 200 mg MR-FORM3 tablets) significantly simplifying the process train.

TABLE 3
Comparative pK Data in Humans for the 100 mg MR-FORM1,100 mg MR-FORM2, and
200 mg MR-FORM 2 Tablets

| | 4 ×100 mg | 2 × 200 mg | 1 × 100 mg | 1 × 100 mg | 2 × 200 mg | 1 × 100 mg |
|---|---|---|---|---|---|---|
| Parameter | MR-FORM 1 | MR-FORM2 | MR-FORM1 | MR-FORM2 | MR-FORM2 | MR-FORM2 |
| (Units) | fasted | fasted | fasted | fasted | fed | fed |
| N, n | 24, 24 | 23, 21 | 23, 19 | 24, 21 | 10, 8 | 12, 12 |
| AUC_{inf} | 1558 (49) | 1478 (53) | 671.8 (59) | 581.3 (61) | 2201 (58) | 797.4 (47) |
| (ng · hr/mL) | | | | | | |
| AUCₗₐₛₜ | 1520 (49) | 1420 (51) | 662.9 (56) | 579.4 (59) | 1914 (65) | 776.4 (49) |
| (ng · hr/mL) | | | | | | |
| Cₘₐₓ (ng/mL) | 76.73 (50) | 68.34 (57) | 39.05 (43) | 35.49 (45) | 239.7 (55) | 83.34 (66) |
| Tₘₐₓ (hr) | 8.01 | 8.00 | 10.0 | 9.00 | 4.20 | 4.01 |
| | (2.00-24.0) | (2.00-24.0) | (2.00-24.0) | (2.00-24.0) | (2.00-6.02) | (2.00-6.03) |
| t_{1/2} (hr) | 12.38 ± 5.34 | 10.49 ± 2.82 | 8.538 ± 2.28 | 9.401 ± 2.61 | 12.28 ± 3.17 | 11.84 ± 4.32 |
| CL/F (L/hr) | 256.8 (49) | 270.6 (53) | 148.9 (59) | 171.9 (61) | 182.0 (58) | 125.3 (47) |
| V_{z}/F (L) | 4238 (54) | 3964 (47) | 1779 (54) | 2243 (50) | 3102 (68) | 1999 (54) |
| T_{lag} (hr) | 0.000 | 0.000 | 0.000 | 0.000 | 0.000 | 0.000b |
| AUC_{inf}(dn) | 3.894 (49) | 3.695 (53) | 6.718 (59) | 5.813 (61) | 5.499 (58) | 7.974 (47) |
| (ng · hr/mL/mg) | | | | | | |
| AUCₗₐₛₜ(dn) | 3.799 (49) | 3.550 (51) | 6.629 (56) | 5.794 (59) | 4.789 (65) | 7.764 (49) |
| (ng · hr/mL/mq) | | | | | | |
| Cₘₐₓ(d n) | 0.1919 (50) | 0.1710 (57) | 0.3905 (43) | 0.3549 (45) | 0.6003 (55) | 0.8334 (66) |
| (ng/mL/mg) | | | | | | |
| t_{1/2} (hr) | 12.38 ± 5.34 | 10.49 ± 2.82 | 8.538 ± 2.28 | 9.401 ± 2.61 | 12.28 ± 3.17 | 11.84 ± 4.32 |
| CL/F (L/hr) | 256.8 (49) | 270.6 (53) | 148.9 (59) | 171.9 (61) | 182.0 (58) | 125.3 (47) |
| V_{z}/F (L) | 4238 (54) | 3964 (47) | 1779 (54) | 2243 (50) | 3102 (68) | 1999 (54) |

N is the number of subjects in the treatment group; n is the number of subjects where t_{1/2}, AUC_{inf}, AUC_{inf}(dn), CL/F and V_{z}/F were determined.

Cₘₐₓ means the maximum observed concentration of 1-(((2S,3S,4S)-3-ethyl-4-fluoro-5-oxopyrrolidin-2-yl)methoxy)-7-methoxyisoquinoline-6-carboxamide.

Tₘₐₓ means the time for Cₘₐₓ.

T_{lag} means the lag time before the start of the absorption phase for 1-(((2S,3S,4S)-3-ethyl-4-fluoro-5-oxopyrrolidin-2-yl)methoxy)-7-methoxyisoquinoline-6-carboxamide.

T_{1/2} means the terminal half-life for 1-(((2S,3S,4S)-3-ethyl-4-fluoro-5-oxopyrrolidin-2-yl)methoxy)-7-methoxyisoquinoline-6-carboxamide calculated as Ln(2)/kₑ₁, where kₑ₁ is the terminal phase rate constant calculated by a linear regression of the log-linear concentration-time curve.

AUC_{inf} means the area under the plasma concentration-time profile for 1-(((2S,3S,4S)-3-ethyl-4-fluoro-5-oxopyrrolidin-2-yl)methoxy)-7-methoxyisoquinoline-6-carboxamide from time 0 extrapolated to infinite time calculated using the formula AUCₗₐₛₜ+(Cₗₐₛₜ/kₑ₁), where Cₗₐₛₜ is the predicted plasma concentration at the last quantifiable time point estimated from the log-linear regression analysis.

AUCₗₐₛₜ means the area under the plasma concentration-time profile for 1-(((2S,3S,4S)-3-ethyl-4-fluoro-5-oxopyrrolidin-2-yl)methoxy)-7-methoxyisoquinoline-6-carboxamide from time 0 to the time of the last quantifiable concentration (Cₗₐₛₜ) calculated using the Linear/Log trapezoidal method.

CL/F means the apparent oral clearance of 1-(((2S,3S,4S)-3-ethyl-4-fluoro-5-oxopyrrolidin-2-yl)methoxy)-7-methoxyisoquinoline-6-carboxamide calculated using the formula Dose/AUC_{inf}.

V_{z}/F means the apparent volume of distribution for 1-(((2S,3S,4S)-3-ethyl-4-fluoro-5-oxopyrrolidin-2-yl)methoxy)-7-methoxyisoquinoline-6-carboxamide calculated using the formula Dose/(AUC_{inf}×kₑ₁).

Cₘₐₓ(dn) means dose normalized Cₘₐₓ for 1-(((2S,3S,4S)-3-ethyl-4-fluoro-5-oxopyrrolidin-2-yl)methoxy)-7-methoxyisoquinoline-6-carboxamide calculated using the formula Cₘₐₓ/dose.

AUC_{inf}(dn) means dose normalized AUC_{inf} for 1-(((2S,3S,4S)-3-ethyl-4-fluoro-5-oxopyrrolidin-2-yl)methoxy)-7-methoxyisoquinoline-6-carboxamide calculated using the formula AUC_{inf}/dose.

AUCₗₐₛₜ(dn) means dose normalized AUCₗₐₛₜ for 1-(((2S,3S,4S)-3-ethyl-4-fluoro-5-oxopyrrolidin-2-yl)methoxy)-7-methoxyisoquinoline-6-carboxamide calculated using the formula AUCₗₐₛₜ/dose.

The relative bioavailability of 100 mg MR-FORM2 fasted compared to 100 mg MR-FORM1 fasted were 81.41%, 85.32% and 90.75% for dose normalized AUC_{inf}, AUCₗₐₛₜ and Cₘₐₓ, respectively. The relative bioavailability of 400 mg MR-FORM2 (2×200 mg MR-FORM2 tablets) fasted compared to 400 mg MR-FORM1 (4×100 mg MR-FORM1) fasted were 96.32%, 95.41% and 88.96% for dose normalized AUC_{inf}, AUCₗₐₛₜ and Cₘₐₓ, respectively.

Compared to the exposures of 1-(((2S,3S,4S)-3-ethyl-4-fluoro-5-oxopyrrolidin-2-yl)methoxy)-7-methoxyisoquinoline-6-carboxamide in fasted state, the exposures of 1-(((2S,3S,4S)-3-ethyl-4-fluoro-5-oxopyrrolidin-2-yl)methoxy)-7-methoxyisoquinoline-6-carboxamide increased when administered with high-fat meal. The dose normalized AUC_{inf}, AUCₗₐₛₜ and Cₘₐₓ of MR-FORM2 100 mg administered with high-fat meal compared to these in fasted state were 127.26%, 125.55% and 240.84%, respectively. Similarly at 400 mg, the dose normalized AUC_{inf}, AUCₗₐₛₜ and Cₘₐₓ of MR-FORM2 administered with high-fat meal compared to these in fasted state were 159.81%, 147.72% and 340.55%, respectively.

### Drug-Drug Interactions

A combination oral pharmacokinetic study was performed between 1-((2S,5R)-5-((7H-pyrrolo[2,3-d]pyrimidin-4-yl)amino)-2-methylpiperidin-1-yl)prop-2-en-1-one and 1-(((2S,3S,4S)-3-ethyl-4-fluoro-5-oxopyrrolidin-2-yl)methoxy)-7-methoxyisoquinoline-6-carboxamide in rat based on the potential for the risk of CYP3A4 time dependent inhibition with 1-((2S,5R)-5-((7H-pyrrolo[2,3-d]pyrimidin-4-yl)amino)-2-methylpiperidin-1-yl)prop-2-en-1-one and the predominance of CYP3A4 clearance with 1-(((2S,3S,4S)-3-ethyl-4-fluoro-5-oxopyrrolidin-2-yl)methoxy)-7-methoxyisoquinoline-6-carboxamide. Doses of 1-((2S,5R)-5-((7H-pyrrolo[2,3-d]pyrimidin-4-yl)amino)-2-methylpiperidin-1-yl)prop-2-en-1-one were 10 and 50 mg/kg and 1-(((2S,3S,4S)-3-ethyl-4-fluoro-5-oxopyrrolidin-2-yl)methoxy)-7-methoxyisoquinoline-6-carboxamide were 30 and 100 mg/kg in male Wistar Han. There were no significant effects of 1-(((2S,3S,4S)-3-ethyl-4-fluoro-5-oxopyrrolidin-2-yl)methoxy)-7-methoxyisoquinoline-6-carboxamide on the pharmacokinetics of 1-((2S,5R)-5-((7H-pyrrolo[2,3-d]pyrimidin-4-yl)amino)-2-methylpiperidin-1-yl)prop-2-en-1-one.

The 10 and 50 mg/kg 1-((2S,5R)-5-((7H-pyrrolo[2,3-d]pyrimidin-4-yl)amino)-2-methylpiperidin-1-yl)prop-2-en-1-one dose dependently increased 1-(((2S,3S,4S)-3-ethyl-4-fluoro-5-oxopyrrolidin-2-yl)methoxy)-7-methoxyisoquinoline-6-carboxamide concentrations at 30 mg/kg (AUC ratios of 1.6 and 3.3 respectively) and 100 mg/kg (AUC ratios of 2.3 and 5.9 respectively).

No significant interactions were found between tofacitinib and 1-(((2S,3S,4S)-3-ethyl-4-fluoro-5-oxopyrrolidin-2-yl)methoxy)-7-methoxyisoquinoline-6-carboxamide. In a 13 week toxicology study in rat, there were no significant effects of 10 mg/kg tofacitinib on either 50 mg/kg 1-(((2S,3S,4S)-3-ethyl-4-fluoro-5-oxopyrrolidin-2-yl)methoxy)-7-methoxyisoquinoline-6-carboxamide in female rats or 100 mg/kg 1-(((2S,3S,4S)-3-ethyl-4-fluoro-5-oxopyrrolidin-2-yl)methoxy)-7-methoxyisoquinoline-6-carboxamide in male rats. Nor were there any significant exposure changes precipitated by 1-(((2S,3S,4S)-3-ethyl-4-fluoro-5-oxopyrrolidin-2-yl)methoxy)-7-methoxyisoquinoline-6-carboxamide on tofacitinib.

### BioMAP Data

The BioMAP systems consist of human primary cell-based systems designed to model different aspects of the human body in an in vitro format. The 12 systems tested characterized the test compounds across a broad set of systems modeling various human disease states. BioMAP systems were constructed with one or more primary cell types from healthy human donors, with stimuli (such as cytokines or growth factors) added to capture relevant signaling networks that naturally occur in human tissue or pathological conditions. Vascular biology was modeled in both a Th1 (3C system) and a Th2 (4H system) inflammatory environment, as well as in a Th1 inflammatory state specific to arterial smooth muscle cells (CASM3C system). Additional systems recapitulated aspects of the systemic immune response that included monocyte-driven Th1 inflammation (LPS system) or T cell stimulation (SAg system), chronic Th1 inflammation driven by macrophage activation (IMphg system) and the T cell-dependent activation of B cells that occurs in germinal centers (BT system). The BE3C system (Th1) and the BF4T system (Th2) represented airway inflammation of the lung, while the MyoF system modeled myofibroblast-lung tissue remodeling. Lastly, skin biology was addressed in the KF3CT system modeled Th1 cutaneous inflammation and the HDF3CGF system modeled wound healing.

All cells were from a pool of multiple donors (n=2-6), commercially purchased and handled according to the recommendations of the manufacturers. Human blood derived CD14+ monocytes were differentiated into macrophages in vitro before being added to the IMphg system. Abbreviations were used as follows: human umbilical vein endothelial cells (HUVEC), peripheral blood mononuclear cells (PBMC), human neonatal dermal fibroblasts (HDFn), B cell receptor (BCR), T cell receptor (TCR) and toll-like receptor (TLR). Cell types and stimuli used in each system were as follows:

3C system [HUVEC+(IL-1β, TNFα and IFNγ)];
4H system [HUVEC+(IL-4 and histamine)];
LPS system [PBMC and HUVEC+LPS (TLR4 ligand)];
SAg system [PBMC and HUVEC+T cell receptor (TCR) ligands];
BT system [CD19+B cells and PBMC+(α-IgM and TCR ligands)];
BF4T system [bronchial epithelial cells and HDFn+(TNFα and IL-4)];
BE3C system [bronchial epithelial cells+(IL-1p, TNFα and IFNγ)];
CASM3C system [coronary artery smooth muscle cells+(IL-1p, TNFα and IFNγ)];
HDF3CGF system [HDFn+(IL-1β, TNFα, IFNγ, EGF, bFGF and PDGF-BB)];
KF3CT system [keratinocytes and HDFn+(IL-1β, TNFα and IFNγ)];
MyoF system [differentiated lung myofibroblasts+(TNFα and TGFβ)]; and
IMphg system [HUVEC and M1 macrophages+Zymosan (TLR2 ligand)].

Systems were derived from either single cell types or co-culture systems. Adherent cell types were cultured in 96 or 384-well plates until confluence, followed by the addition of PBMC (SAg and LPS systems). The BT system consisted of CD19+B cells co-cultured with PBMC and stimulated with a BCR activator and low levels of TCR stimulation. Test agents were prepared in DMSO (final concentration <0.1%) and were added 1 hour before stimulation, and remained in culture for 24 hours or as otherwise indicated (48-hrs, MyoF system; 72-hrs, BT system (soluble readouts); 168-hrs, BT system (secreted IgG)). Each plate contained drug controls (e.g., legacy control test agent colchicine at 1.1 μM), negative controls (e.g., non-stimulated conditions) and vehicle controls (e.g., 0.1% DMSO) appropriate for each system. Direct ELISA was used to measure biomarker levels of cell-associated and cell membrane targets. Soluble factors from supernatants were quantified using either HTRF® detection, bead-based multiplex immunoassay or capture ELISA. Overt adverse effects of test agents on cell proliferation and viability (cytotoxicity) were detected by sulforhodamine B (SRB) staining, for adherent cells, and alamarBlue® reduction for cells in suspension. For proliferation assays, individual cell types were cultured at subconfluence and measured at time points optimized for each system (48-hrs: 3C and CASM3C systems; 72-hrs: BT and HDF3CGF systems; 96-hrs: SAg system). Cytotoxicity for adherent cells was measured by SRB (24-hrs: 3C, 4H, LPS, SAg, BF4T, BE3C, CASM3C, HDF3CGF, KF3CT, and/Mphg systems; 48-hrs: MyoF system), and by alamarBlue staining for cells in suspension (24-hrs: SAg system; 42-hrs: BT system) at the time points indicated.

Each tested compound generated a signature BioMAP profile that was created from the changes in protein biomarker readouts within individual system environments. Biomarker readouts (7-17 per system) were selected for therapeutic and biological relevance, predicted disease outcomes or specific drug effects and were validated using agents with known mechanism of action (MoA). Each readout was measured quantitatively by immune-based methods that detected protein (e.g., ELISA) or functional assays that measured proliferation and viability. BioMAP readouts were diverse and included cell surface receptors, cytokines, chemokines, matrix molecules and enzymes. In total, the BioMAP systems contained 148 biomarker readouts that captured biological changes that occur within the physiological context of the particular BioMAP system.

Biomarker measurements in a test agent-treated sample were divided by the average of control samples (at least 6 vehicle controls from the same plate) to generate a ratio that was then log₁₀ transformed. Significance prediction envelopes were calculated using historical vehicle control data at a 95% confidence interval.

Biomarker activities were annotated when 2 or more consecutive concentrations change in the same direction relative to vehicle controls, were outside of the significance envelope and had at least one concentration with an effect size >20% (|log₁₀ ratio|>0.1). Biomarker key activities were described as modulated if these activities increased in some systems, but decreased in others. Cytotoxic conditions were noted when total protein levels decrease by more than 50% (log₁₀ ratio of SRB or alamarBlue levels <−0.3) and were indicated by a thin black arrow above the X-axis. A compound was considered to have broad cytotoxicity when cytotoxicity was detected in 3 or more systems. Concentrations of test agents with detectablet broad cytotoxicity were excluded from biomarker activity annotation and downstream benchmarking, similarity search and cluster analysis. Antiproliferative effects were defined by an SRB or alamarBlue log₁₀ ratio value <−0.1 from cells plated at a lower density and were indicated by grey arrows above the X-axis. Cytotoxicity and antiproliferative arrows only required one concentration to meet the indicated threshold for profile annotation.

### 1-(((2S,3S,4S)-3-Ethyl-4-fluoro-5-oxopyrrolidin-2-yl)methoxy)-7-methoxyisoquinoline-6-carboxamide BioMAP Data

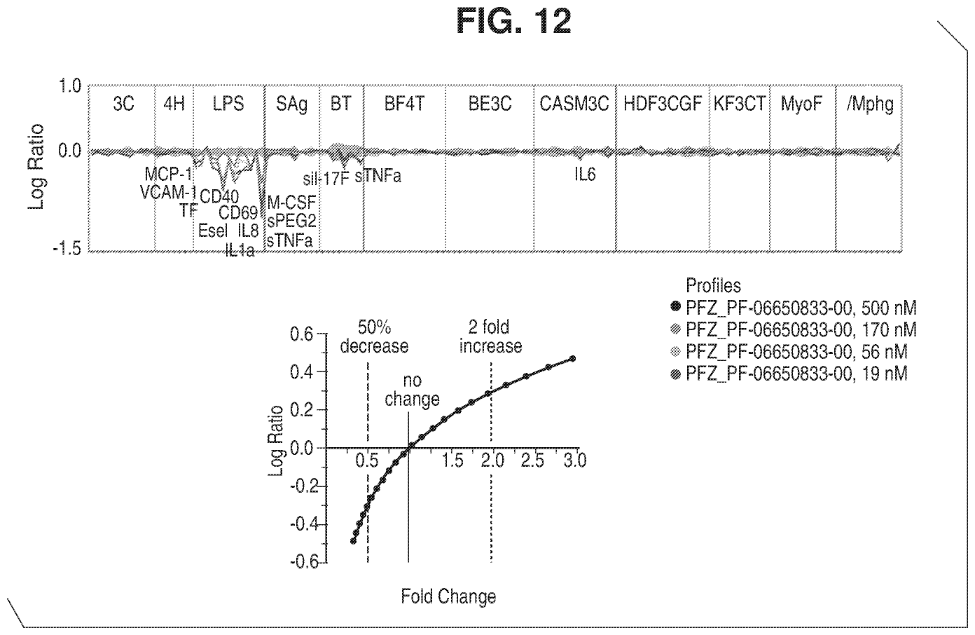

The BioMAP results (FIG. 12) indicated that 1-(((2S,3S,4S)-3-ethyl-4-fluoro-5-oxopyrrolidin-2-yl)methoxy)-7-methoxyisoquinoline-6-carboxamide specifically inhibited Th1 and innate immune responses from PBMCs induced by TLR stimulation (LPS system) including the production of inflammatory mediators PGE₂, TNFα, IL-1α, and MCP1. 1-(((2S,3S,4S)-3-Ethyl-4-fluoro-5-oxopyrrolidin-2-yl)methoxy)-7-methoxyisoquinoline-6-carboxamide had little effect on T-cell dependent activation of B-cells (BT system). 1-(((2S,3S,4S)-3-Ethyl-4-fluoro-5-oxopyrrolidin-2-yl)methoxy)-7-methoxyisoquinoline-6-carboxamide was active in 14 annotated readouts with no evidence of overt cytotoxicity on multiple primary human cell types stimulated in 12 tissue and disease models. 1-(((2S,3S,4S)-3-Ethyl-4-fluoro-5-oxopyrrolidin-2-yl)methoxy)-7-methoxyisoquinoline-6-carboxamide had no antiproliferative effect on any of the primary cell types. 1-(((2S,3S,4S)-3-Ethyl-4-fluoro-5-oxopyrrolidin-2-yl)methoxy)-7-methoxyisoquinoline-6-carboxamide impacted inflammation-related activities (decreased E-selectin, MCP-1, VCAM-1, IL-6, IL-8, IL-1α, secreted TNFα, secreted PGE₂), immunomodulatory activities (decreased CD40, CD69, M-CSF, secreted IL-17F), and hemostasis-related activities (decreased TF).

1-(((2S,3S,4S)-3-Ethyl-4-fluoro-5-oxopyrrolidin-2-yl)methoxy)-7-methoxyisoquinoline-6-carboxamide was primarily active across all tested concentrations in the LPS system modeling monocyte activation responses. The LPS system data coupled with the data from the/Mphg system containing CD14+ derived macrophages, where no activity was found, revealed a high degree of selectivity for impacting monocyte but not macrophage responses. 1-(((2S,3S,4S)-3-Ethyl-4-fluoro-5-oxopyrrolidin-2-yl)methoxy)-7-methoxyisoquinoline-6-carboxamide selectively blocked immune activation mediated by monocytes but did not block immune activation mediated by macrophage-types.

Additional activities of 1-(((2S,3S,4S)-3-ethyl-4-fluoro-5-oxopyrrolidin-2-yl)methoxy)-7-methoxyisoquinoline-6-carboxamide included modest effects in BT and CASM3C systems and concentration-related decreases in secreted IL-17F and inhibition of secreted TNFα and IL-6 at the higher two concentrations only.

Together the data showed that 1-(((2S,3S,4S)-3-ethyl-4-fluoro-5-oxopyrrolidin-2-yl)methoxy)-7-methoxyisoquinoline-6-carboxamide strongly and selectively inhibited monocyte activation responses in a human primary cell based model of vascular inflammation biology. These activities may be highly relevant for development of 1-(((2S,3S,4S)-3-ethyl-4-fluoro-5-oxopyrrolidin-2-yl)methoxy)-7-methoxyisoquinoline-6-carboxamide as a therapy for innate inflammation diseases with largely unmet needs such as the auto-immune indications COPD and IBD.

TABLE 4
1-(((2S,3S,4S)-3-Ethyl-4-fluoro-5-oxopyrrolidin-2-yl)methoxy)-7-
methoxyisoquinoline-6-carboxamide BioMAP Profile

| Biological and Disease | Decreased |
|---|---|
| Relevance Category | Activity |
| Inflammation-related activities | MCP-1, VCAM-1, E-selectin, IL-6, IL-8, IL-1α, *sTNFα, *sPGE₂ |
| Immunomodulatory activities | CD40, CD69, M-CSF, *sIL-17F |
| Hemostasis-related activities | TF |

*s means secreted

1-(((2S,3S,4S)-3-Ethyl-4-fluoro-5-oxopyrrolidin-2-yl)methoxy)-7-methoxyisoquinoline-6-carboxamide inhibited MCP-1, VCAM-1, E-selectin, IL-8, IL-1a, sTNFa, sPGE2, CD40, CD69, M-CSF, and TF in the LPS system and showed modest inhibition of IL-6 and sIL-17F in the BT system.

Monocyte chemoattractant protein-1 (MCP-1) is a chemoattractant cytokine (i.e. chemokine) that regulates recruitment of monocytes and T cells into sites of inflammation. MCP-1 in the LPS system is regulated by the HDAC, histamine H1R, IKK2 and p38 MAPK pathways.

Vascular Cell Adhesion Molecule 1 (VCAM-1) is a cell adhesion molecule that mediates adhesion of monocytes and T cells to endothelial cells. VCAM-1 in the LPS system is regulated by the HDAC and IKK2 pathways.

E-Selectin is a cell adhesion molecule expressed only on endothelial cells that mediates leukocyte-endothelial cell interactions. E-Selectin in the LPS system is regulated by the HDAC, IKK2, and p38 MAPK pathways.

Interleukin-8 (IL-8) is a chemokine that mediates neutrophil recruitment into acute inflammatory sites. IL-8 in the LPS system is regulated by the IKK and p38 MAPK pathways.

Interleukin-1 alpha (IL-1α) is a secretory pro-inflammatory cytokine involved in endothelial cell activation and neutrophil recruitment. IL-1a in the LPS system is regulated by the HDAC, HMG-CoA Reductase, IKK2, and p38 MAPK pathways.

Tumor necrosis factor alpha (TNFα) is a secreted pro-inflammatory cytokine involved in Th1 vascular inflammation. Secreted TNFα (sTNFα) is regulated in the LPS system by the EGFR, glucocorticoid receptor, HDAC, histamine H1R, IKK2, PDE4, PI3K, PKC, RAR/RXR, Src, vitamin D receptor, and p38 MAPK.

Prostaglandin E₂ (PGE₂) is an immunomodulatory lipid mediator involved in muscle contractility, inflammation pain and kidney function. Secreted PGE₂ (sPGE₂) in the LPS system is regulated by the following pathways: IKK2, MEK, PKC, RAR/RXR, vitamin D receptor, mTOR, and p38 MAPK.

MCP-1, VCAM-1, CD40, E-selectin, IL-8, IL-1α, sTNFα, and sPGE₂ are associated with inflammatory activities in the LPS system modeling monocyte Th1 vascular inflammation.

CD40 is a cell surface adhesion receptor and costimulatory receptor for T cell activation that is expressed on antigen presenting cells, endothelial cells, smooth muscle cells, fibroblasts and epithelial cells. CD40 is regulated in the LPS system by the histamine H1R, IKK2, PI3K, RAR/RXR, Src, and mTOR pathways.

CD69 is a cell surface activation antigen regulated in the LPS system by the histamine H1R and IKK2 pathways.

CD40 and CD69 are categorized as immunomodulatory-related activities in the LPS system modeling monocyte-driven Th1 vascular inflammation.

Macrophage colony-stimulating factor (M-CSF) is a secreted and cell surface cytokine that mediates macrophage differentiation. M-CSF is categorized as a tissue remodeling-related activity in the LPS system modeling monocyte-driven Th1 vascular inflammation regulated in the LPS system by the HDAC, IKK2, RAR/RXR, and p38 MAPK pathways.

Tissue Factor (TF) is a cell surface receptor for coagulation of Factor VII that promotes the formation of thrombin during the process of thrombosis and coagulation. Tissue Factor is categorized as a hemostasis-related activity in the LPS system modeling monocyte-driven Th1 vascular inflammation and is regulated in the LPS system by the IKK2 and p38 MAPK pathways.

Interleukin-17F (IL-17F) is a pro-inflammatory cytokine produced by T cells that induces cytokine, chemokine, and adhesion molecule production and mediates neutrophil recruitment to sites of inflammation. sIL-17 is regulated in the BT system by the calcineurin, EGFR, glucocorticoid receptor, HDAC, HMG-CoA Reductase, histamine H1R, JAK, MEK, microtubule, PKC, RAR/RXR, Src, TNFα, vitamin D receptor, mTOR, and p38 MAPK pathways.

Interleukin-6 (IL-6) is a secreted pro-inflammatory cytokine and acute phase reactant. Secreted IL-6 (sIL-6) is regulated in the BT system by the calcineurin, EGFR, glucocorticoid receptor, HDAC, histamine H1R, IKK2, JAK, MEK, PI3K, PKC, Src, TNFα, vitamin D receptor, mTOR, and p38 MAPK pathways.

Secreted IL-17F and IL-6 are categorized as immunomodulatory-related activities in the BT system modeling T cell dependent B cell activation.

### Tofacitinib BioMAP Data

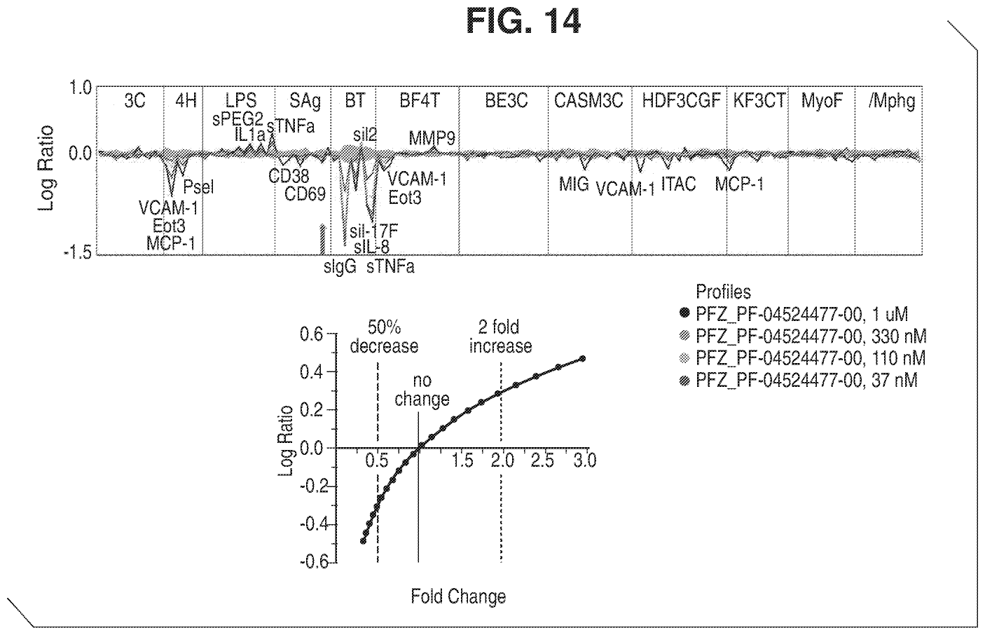

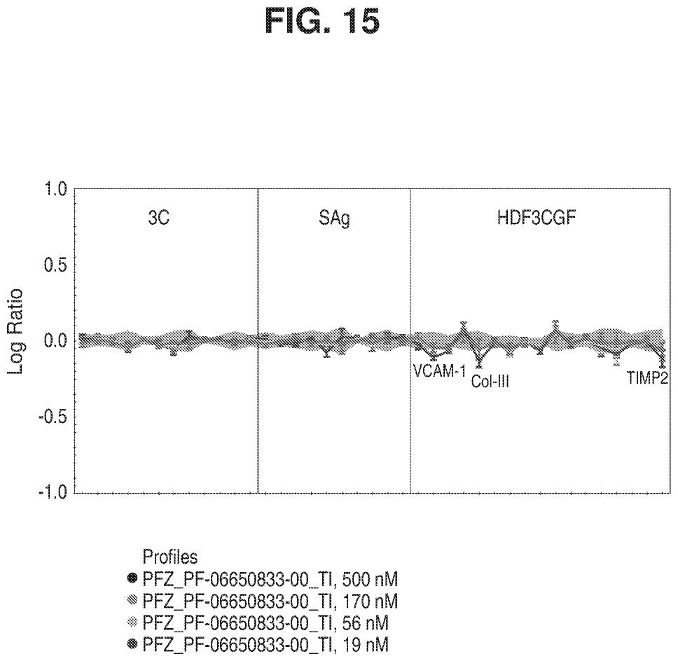

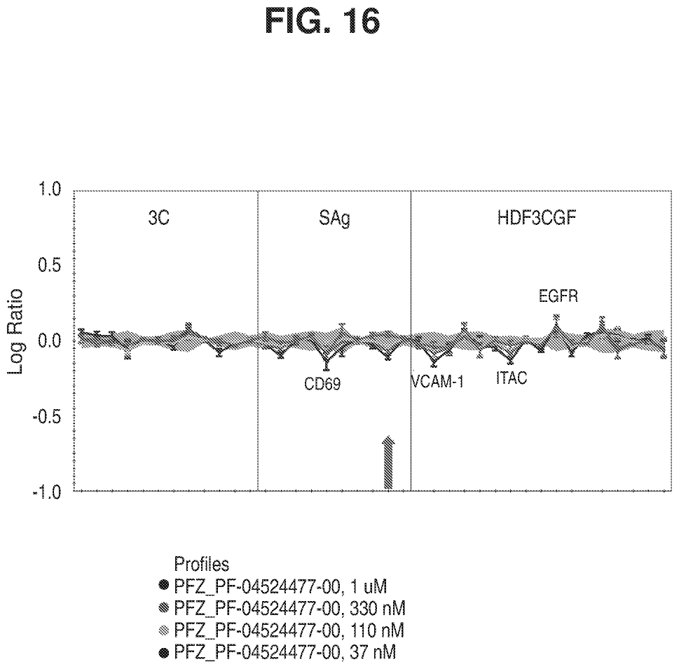

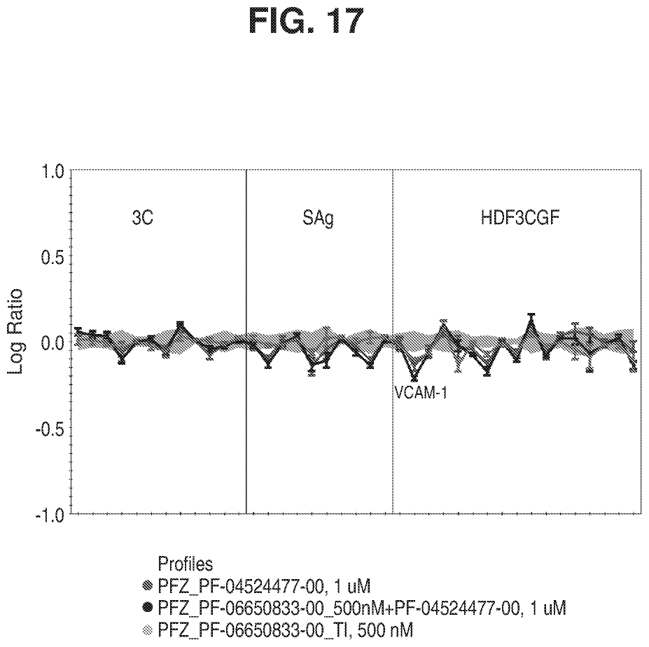

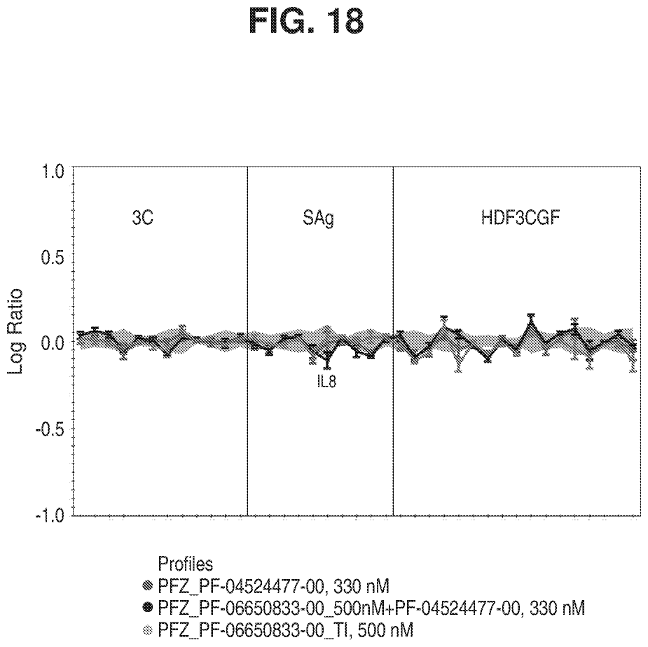

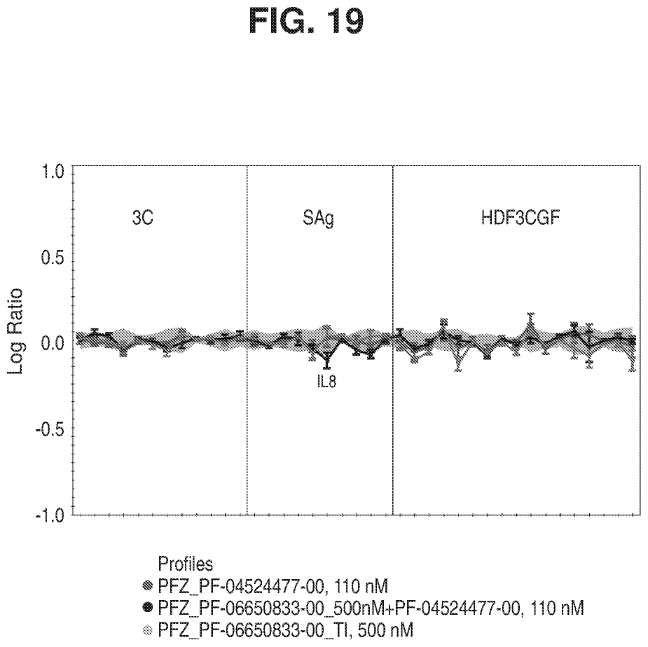

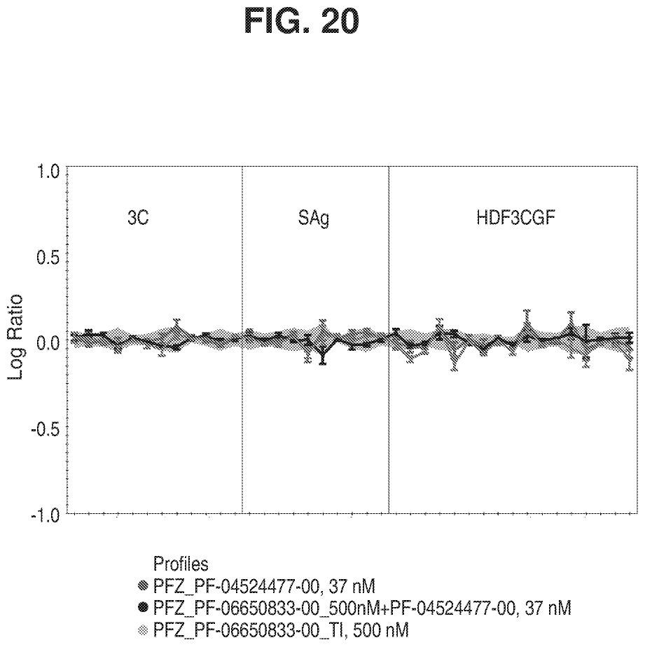

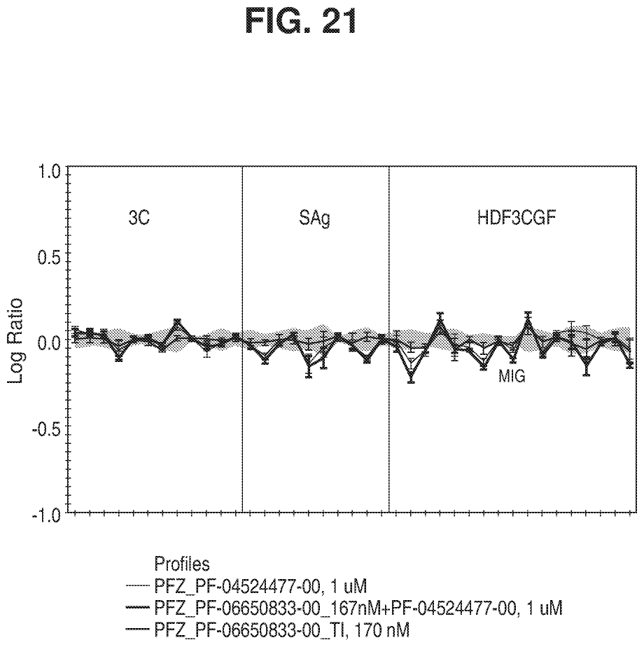

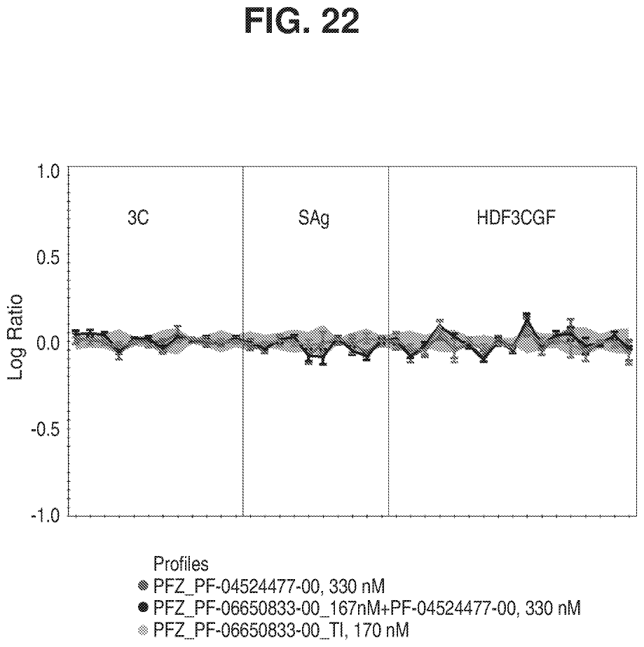

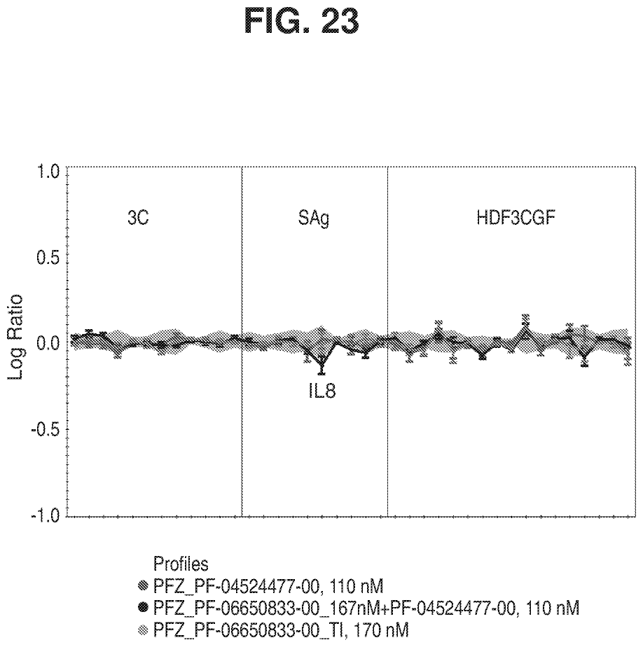

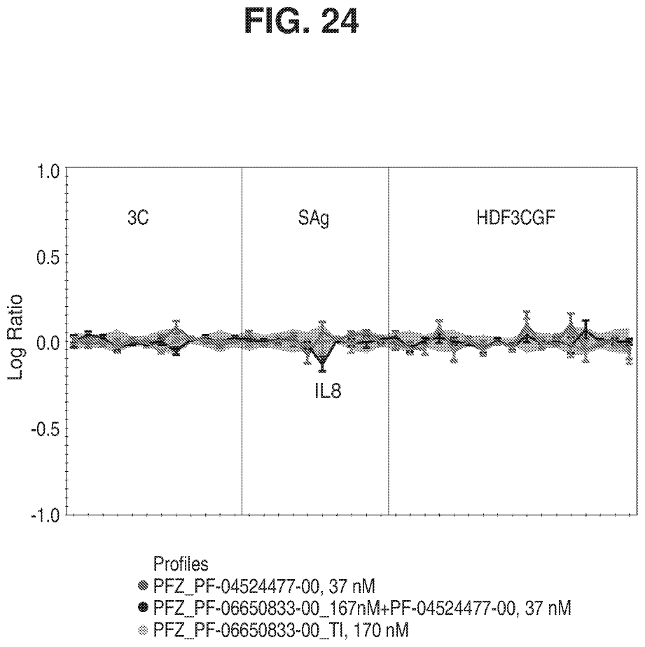

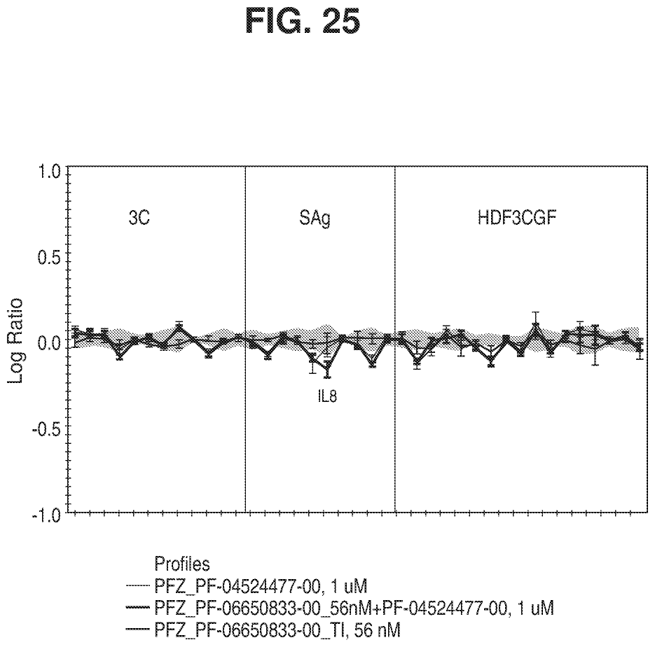

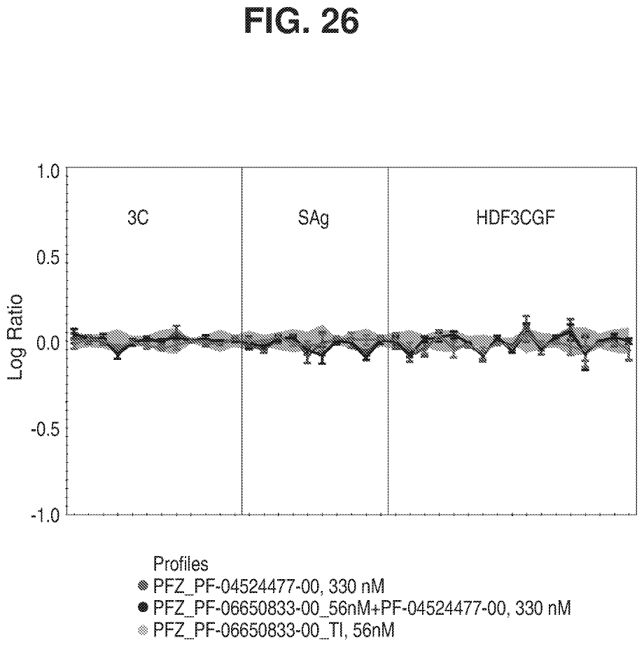

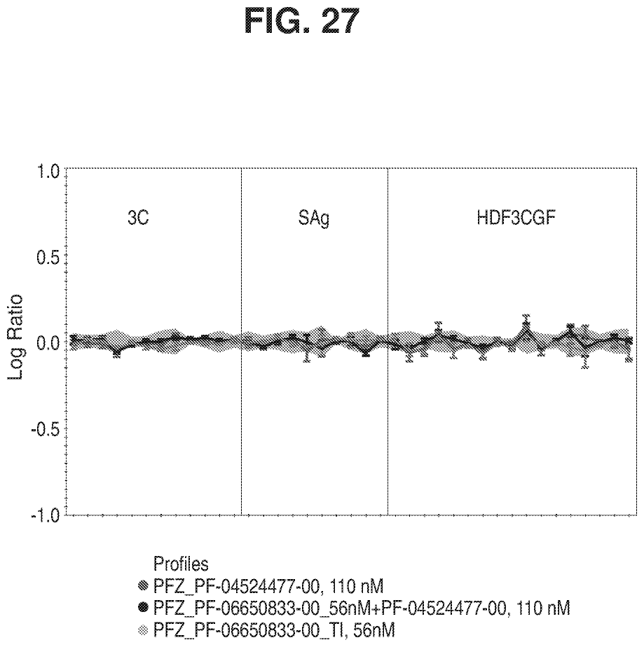

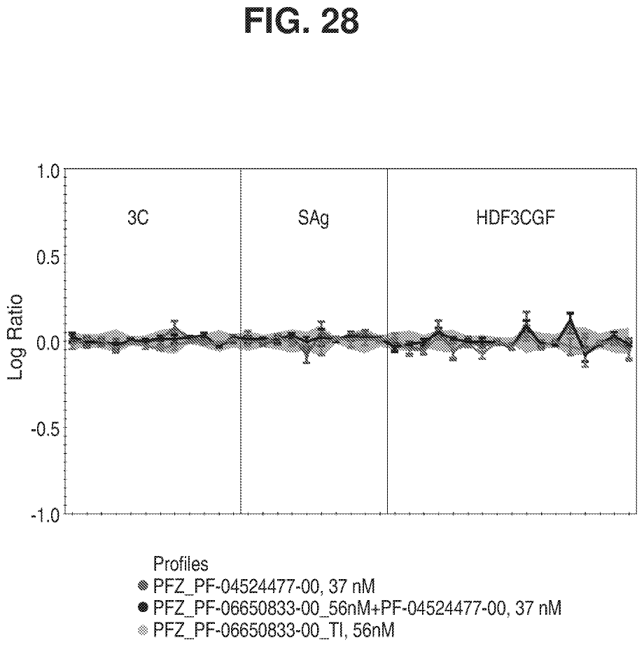

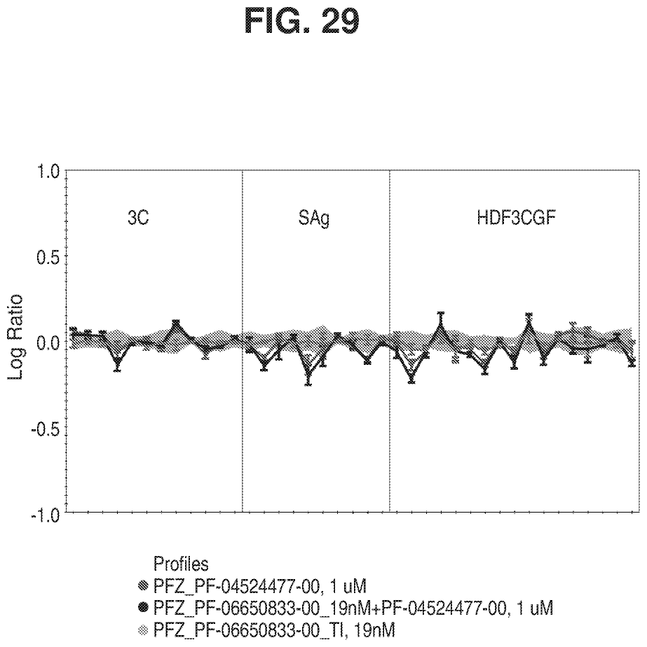

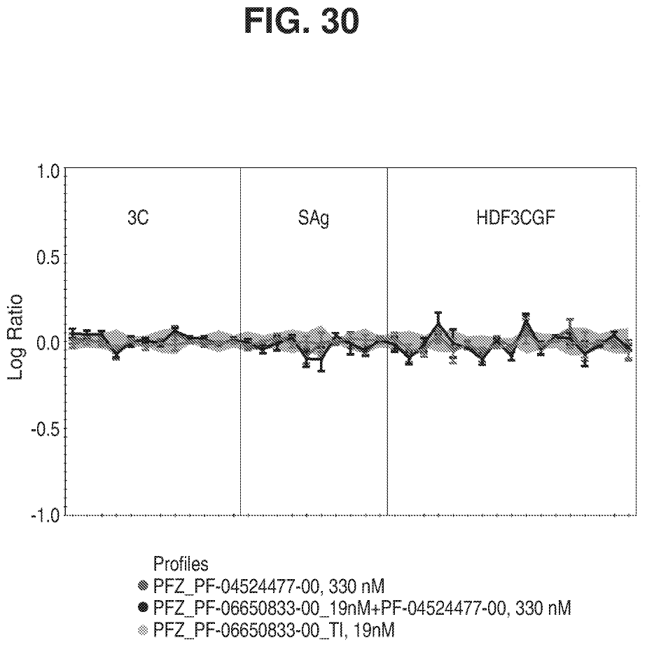

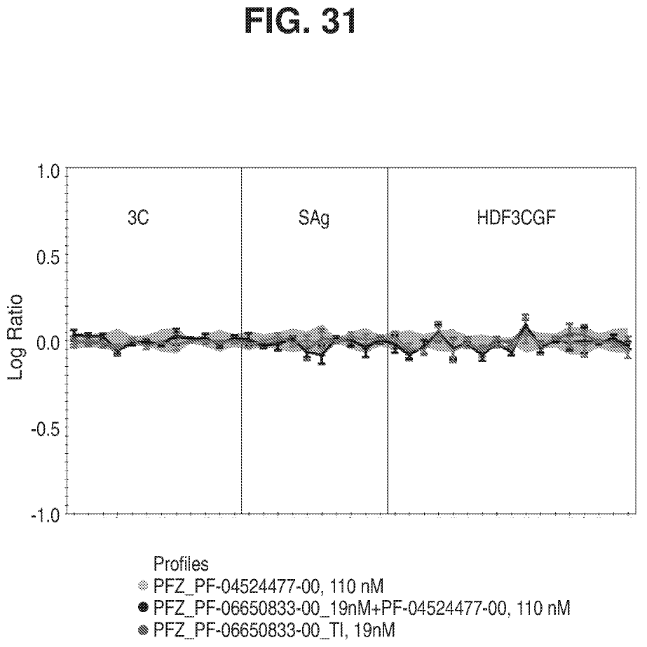

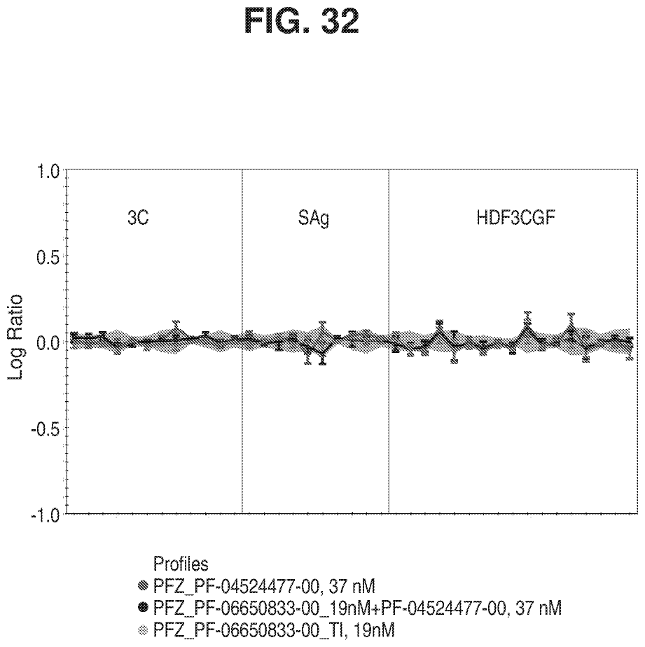

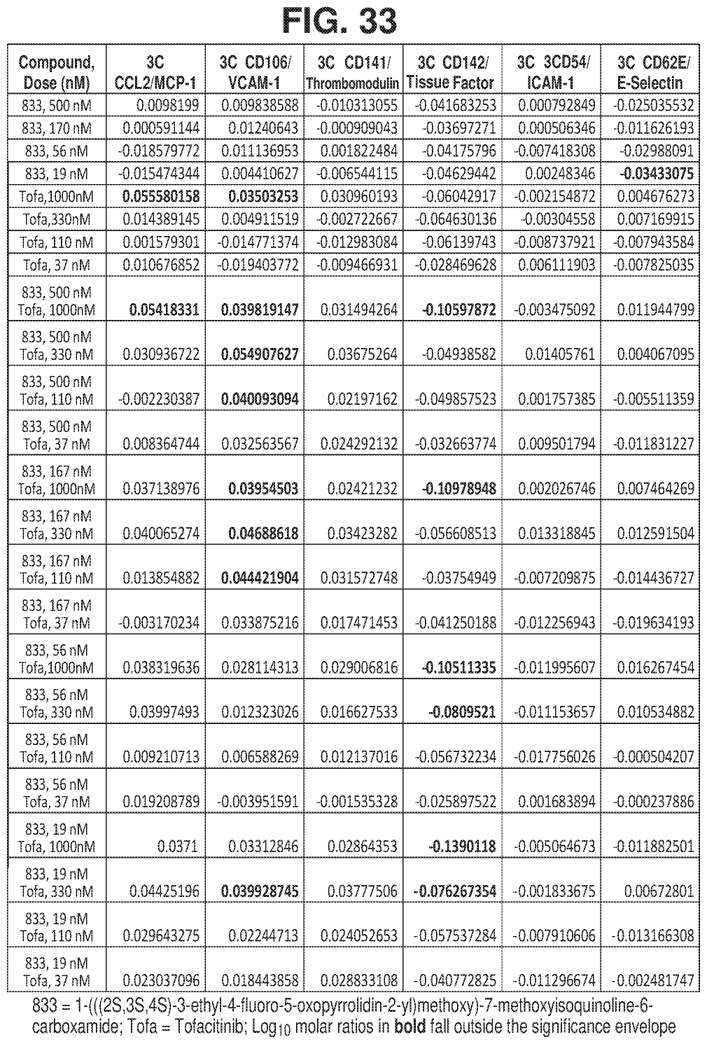

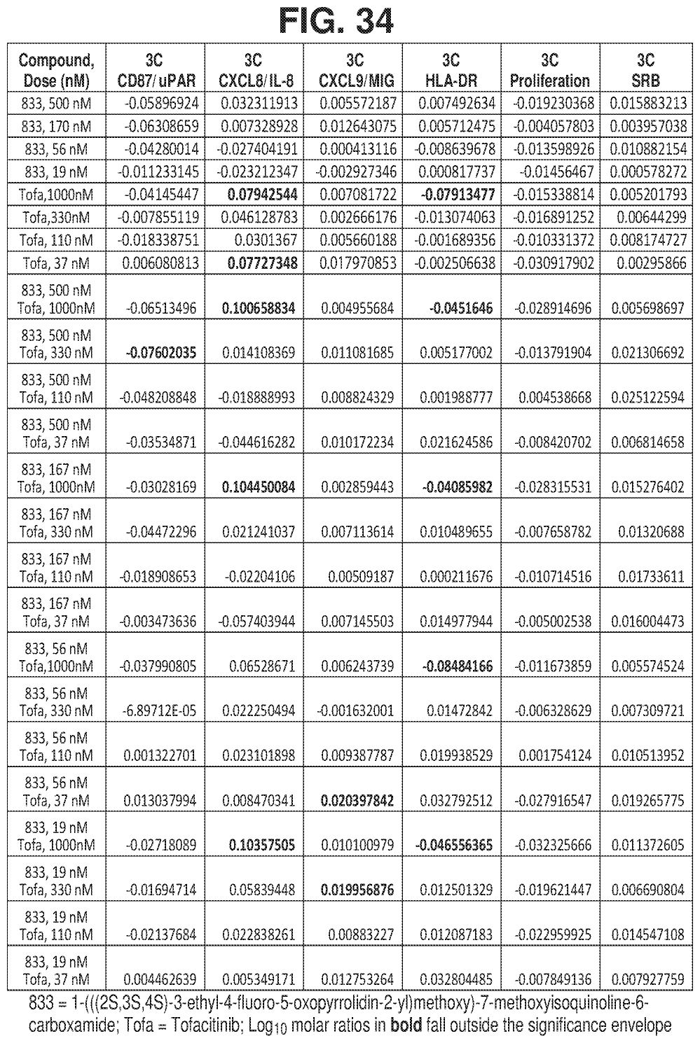

The BioMAP analysis of tofacitinib (FIG. 14) showed activity in 21 annotated readouts with no evidence of overt cytotoxicity on multiple primary human cell types stimulated in 12 tissue and disease models. Tofacitinib was selectively antiproliferative to T cells that mediates IL-2 driven T cell proliferation. Tofacitinib had no cytostatic effect on B cells, vascular cell types or fibroblasts. Tofacitinib impacted inflammation-related activities (decreased Eotaxin 3, MCP-1, VCAM-1, I-TAC, MIG, P-selectin; increased IL-1α, secreted PGE2; and modulated secreted TNFα), immunomodulatory activities (decreased secreted IgG, CD38, secreted IL-6, CD69, secreted IL-17F; increased secreted IL-2), and tissue remodeling activities (increased MMP-9).

Tofacitinib was active over all tested concentrations in the BT system modeling T cell-dependent B cell activation and in two systems modeling IL-4 driven inflammation (4H and BF4T). An increase in secreted IL-2 in the BT system was observed with all concentrations of tofacitinib. This is a biomarker activity seen that may relate to a compensatory feedback effect of blocking cytokine stimulation in this system.

In the LPS system, tofacitinib showed increased levels of IL-1α, secreted PGE₂ and secreted TNFα. In the LPS system, secreted PGE₂ was produced by multiple cell types, including monocytes and endothelial cells.

TABLE 5
Tofacitinib BioMAP Profile

| Biological and Disease | Decreased | Increased | Modulated |
|---|---|---|---|
| Relevance Category | Activity | Activity | Activity |
| Inflammation-related | MCP-1, Eot3, | IL-1α, | *sTNFα |
| activities | VCAM-1, P-selectin, | *sPGE₂ | |
| | ITAC, MIG | | |
| Immunomodulatory | CD38, CD69, *sIgG, | *sIL-2 | |
| activities | *sIL-6, *sIL-17F | | |
| Tissue remodeling activities | | MMP9 | |

*s means secreted

Tofacitinib inhibited: MCP-1, Eot3, VCAM-1, P-selectin in the 4H system; CD38 and CD69 in the SAg system; sIgG, sIL-17F, sIL-6, and sTNFα in the BT system; Eot3, and VCAM-1 in the BF4T system; MIG in the CASM3C system; VCAM-1 and ITAC in the HDF3CGF system; and MCP-1 in the KF3CT system. Tofacitinib stimulated production of: IL-1α, sPEG₂, and sTNFα in the LPS system; sIL-2 in the BT system; and MMP9 in the BF4T system.

Tofacitinib also had antiproliferative activity in the SAg system. Proliferation in the SAg system is a measure of T cell proliferation which is the critical event driving both adaptive immunity as well as many auto-immune diseases such as RA, PsA, MS, and IBD. Proliferation in the SAg system is regulated by the calcineurin, EFFR, HDAC, HMG-CoA Reductase, histamine H1R, IKK2, JAK, MEK, microtubule, PI3K, PKC, RAR/RXR, Src, and mTOR pathways.

Monocyte chemoattractant protein-1 (MCP-1) is a chemoattractant cytokine that regulates the recruitment of monocytes and T cells into sites of inflammation. MCP-1 is regulated in the 4H system by the HMG-CoA Reductase and histamine H1R pathways.

Eotaxin-3 (Eto3) is a chemokine that mediates recruitment of eosinophils and basophils into sites of tissue inflammation. Eto3 is regulated in the 4H system by the HDAC, histamine H1R, IKK2, JAK, and RAR/RXR pathways.

Vascular adhesion molecule 1 (VCAM-1) is a cell adhesion molecule that mediates adhesion of monocytes and T cells to endothelial cells. VCAM-1 is regulated in the 4H system by the calcineurin, HDAC, histamine H1R, IKK2, JAK, and RAR/RXR pathways.

P-Selectin is a cell adhesion molecule that mediates platelet-endothelial cell and leukocyte-endothelial interactions. P-Selectin is regulated in the 4H system by the JAK and PI3K pathways.

MCP-1, Eot3, VCAM-1, P-selectin are categorized as inflammatory-related activities in the 4H system modeling Th2 vascular inflammation.

CD38 is a cell surface enzyme and marker of cell activation that is involved in T cell activation/co-stimulation and chemotaxis. CD38 in the SAg system is regulated by the calcineurin, HDAC, IKK2, JAK, MEK, PI3K, PKC, and RAR/RXR pathways.

CD69 is a cell surface antigen that is induced early during immune activation and is involved in lymphocyte proliferation and activation. CD69 in SAg system is regulated by the calcineurin, EGFR, HMG-CoA Reductase, histamine H1R, IKK2, JAK, MEK, PKC, and Src pathways.

CD38 and CD69 are categorized as immunomodulatory-related activities in the SAg system modeling T cell-driven Th1 vascular inflammation.

Secreted IgG (sIgG) is produced by B cells and is the main type of antibody found in blood and extracellular fluid that mediates response against pathogens. IgG is regulated in the BT system by the calcineurin, EGFR glucocorticoid receptor, HDAC, HMG-CoA Reductase, histamine HiR, IKK2, JAK, microtubule, PDE4, PI3K, PKC, Src, vitamin D receptor, mTOR, and p38 MAPK pathways.

Interleukin-17F (IL-17F) is a pro-inflammatory cytokine produced by T cells that induces cytokine, chemokine, and adhesion molecule production and mediates neutrophil recruitment to sites of inflammation. IL-17F is regulated in the BT system by the calcineurin, EGFR, glucocorticoid receptor, HDAC, HMG-CoA Reductase, histamine H1R, JAK, MEK, microtubule, PKC, RAR/RXR, Src, TNFα, vitamin D receptor, mTOR, and p38 MAPK pathways.

Interleukin-6 (IL-6) is a secreted pro-inflammatory cytokine and acute phase reactant. Secreted IL-6 (sIL-6) is regulated in the BT system by the calcineurin, EGFR, glucocorticoid receptor, HDAC, histamine H1R, IKK2, JAK, MEK, PKC, Src, TNFα, vitamin D receptor, mTOR, and p38 MAPK pathways.

Tumor necrosis factor alpha (TNFα) is a secreted pro-inflammatory cytokine involved in Th1 vascular inflammation. Secreted TNFα (sTNFα) is regulated in the BT system by the calcineurin, EGFR, glucocorticoid receptor, HDAC, HMG-CoA Reductase, histamine H1R, IKK2, JAK, MEK, PI3K, PKC, RAR/RXR, Src, TNFα, vitamin D receptor, and mTOR pathways.

sIgG, sIL-17F, sIL-6, and sTNFα are categorized as inflammatory-related activities in the BT system modeling T cell dependent B cell activation.

Eotaxin3 (Eot3) is a chemokine that mediates recruitment of eosinophils and basophils into tissue sites. Eot3 is regulated in the BF4T system by the EGFR and RAR/RXR pathways.

Vascular cell adhesion molecule 1 (VCAM-1) is a cell adhesion molecule that mediates leukocyte-endothelial cell adhesion and leukocyte recruitment.

Eot3 and VCAM-1 are categorized in the BF4T system as inflammatory-related activities modeling Th2 airway inflammation.

Monokine induced by gamma interferon (MIG) is a chemokine that mediates T cell recruitment. MIG is categorized as an inflammatory-related activity in the CASM3C system modeling Th1 vascular smooth muscle inflammation. MIG is regulated in the CASM3C system by the HDAC, IKK2, and JAK pathways.

Vascular cell adhesion molecule 1 (VCAM-1) is a cell adhesion molecule that mediates monocytes and T cells to endothelial cells. VCAM-1 is regulated in the HDF3CGF system by the IKK2, JAK, MEK, RAR/RXR, Src, and vitamin D receptor.

Interferon-inducible T cell alpha chemoattractant (ITAC) is a chemokine that mediates T cell and monocyte chemotaxis. ITAC is regulated in the HDF3CGF system by the HDAC, JAK, and RAR/RXR pathways.

VCAM-1 and ITAC are categorized as inflammation-related activities in the HDF3CGF system modeling Th1 inflammation involved in wound healing and matrix remodeling of the skin.

Monocyte chemoattractant protein-1 (MCP-1) is a chemoattractant cytokine (chemokine) that regulates the recruitment of monocytes and T cells into sites of inflammation. MCP-1 is categorized as an inflammatory-related activity in the KF3CT system modeling Th1 cutaneous inflammation. MCP-1 is regulated in the KF3CT system by the IKK2 pathway.

Interleukin-1 alpha (IL-1α) is a secretory pro-inflammatory cytokine involved in endothelial cell activation and neutrophil recruitment. IL-1α in the LPS system is regulated by the HDAC, HMG-CoA Reductase, IKK2, and p38 MAPK pathways.

Prostaglandin E₂ (PGE₂) is an immunomodulatory lipid mediator involved in muscle contractility, inflammation pain and kidney function. Secreted PGE₂ (sPGE₂) in the LPS system is regulated by the following pathways: IKK2, MEK, PKC, RAR/RXR, vitamin D receptor, mTOR, and p38 MAPK.

Tumor necrosis factor alpha (TNFα) is a secreted pro-inflammatory cytokine involved in Th1 vascular inflammation. Secreted TNFα (sTNFα) is regulated in the LPS system by the EGFR, glucocorticoid receptor, HDAC, histamine H1R, IKK2, PDE4, PI3K, PKC, RAR/RXR, Src, vitamin D receptor, and p38 MAPK.

IL-1α, sTNFα, and sPGE₂ are associated with inflammatory activities in the LPS system modeling monocyte Th1 vascular inflammation.

Interleukin-2 (IL-2) is a secreted pro-inflammatory cytokine produced by T cells that regulate lymphocyte proliferation and promotes T cell differentiation. Secreted IL-2 (sIL-2) is categorized as an immunomodulatory-related activity in the BT system modeling T cell dependent B cell activation. sIL-2 in the BT system is regulated by the calcineurin, EGFR glucocorticoid receptor, HDAC, histamine H1R, IKK2, JAK, MEK, PI3K, PKC, RAR/RXR, Src, vitamin D receptor, mTOR, and p38 MAPK pathways

Matrix metalloproteinase-9 (MMP-9) is a gelatinase B that degrades collagen IV and gelatin and is involved in airway matrix remodeling. MMP-9 is categorized as a tissue remodeling-related activity in the BF4T system modeling Th2 airway inflammation.

### 1-((2S,5R)-5-((7H-Pyrrolo[2,3-d]pyrimidin-4-yl)amino)-2-methylpiperidin-1-yl)prop-2-en-1-one BioMAP Data

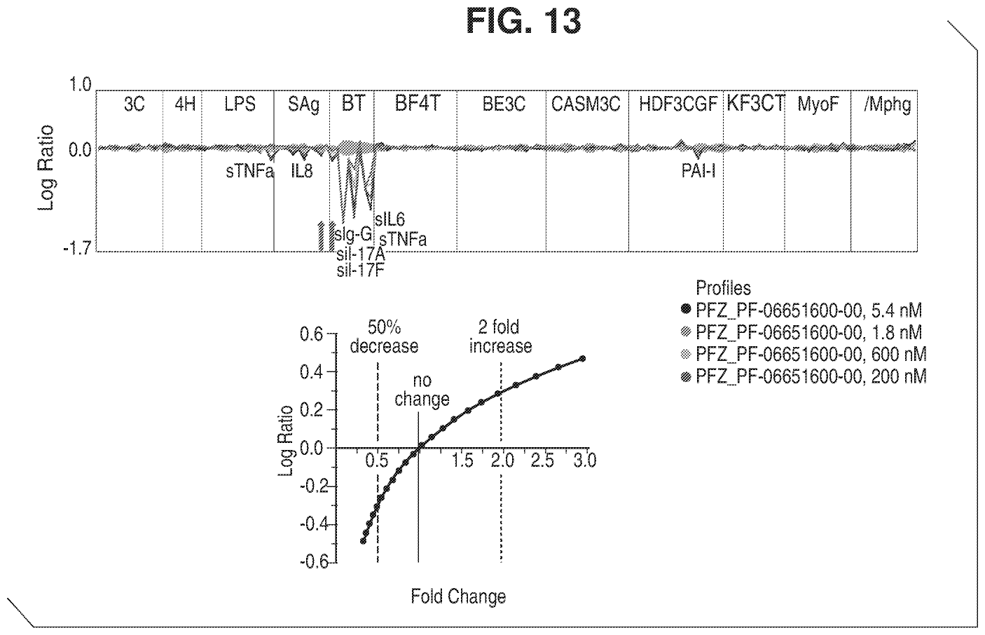

The BioMAP analysis of 1-((2S,5R)-5-((7H-Pyrrolo[2,3-d]pyrimidin-4-yl)amino)-2-methylpiperidin-1-yl)prop-2-en-1-one (FIG. 13) showed inhibition of: TNFα in the LPS system; IL-8 in the SAg system; sIgG, sIL-17A, sIL-17F, sIL-6, and TNFα in the BT system; and PA-I in the HDF3CGF system. The proliferation of T cells was inhibited in the SAg system and B cell proliferation was inhibited in the BT system.

TABLE 6
1-((2S,5R)-5-((7H-pyrrolo[2,3-d]pyrimidin-4-yl)amino)-2-
methylpiperidin-1-yl)prop-2-en-1-one BioMAP Profile

| Biological and Disease | Decreased |
|---|---|
| Relevance Category | Activity |
| Inflammation-related activities | *SIL-17A, *SIL-17F, *sIL-6, IL-8, *sTNFα, |
| Immunomodulatory activities | *sIgG, B cell proliferation, T cell proliferation |
| Tissue Remodeling Activities | PAI-I |

*s means secreted

TNFα is a secreted pro-inflammatory cytokine involved in Th1 vascular inflammation. Secreted TNFα is regulated in the LPS system by the EGFR, glucocorticoid receptoreceptor, HDAC, histamine H1R, IKK2, PDE4, PI3K, PKC, RAR/RXR, Src, vitamin D receptor, and p38 MAPK.

IL-8 is a chemokine that mediates neutrophil recruitment into acute inflammatory sites and is categorized as an inflammation-related activity in the SAg system modeling T cell-driven Th1 vascular inflammation. IL-8 in the SAg system is regulated by the calcineurin, HMG-CoA reductase, IKK2, JAK, MEK, PKC, Src, TNFα, and p38 MAP pathways.

Secreted IgG (sIgG) is produced by B cells and is the main type of antibody found in blood and extracellular fluid that mediates the immune response against pathogens. sIgG in the BT system is regulated by the calcineurin, EGFR, HDAC, HMG-CoA reductase, histamine H1R, IKK2, JAK, microtubule, PDE4, PI3K, PKC, Src, vitamin D receptor, mTOR, and p38 MAP pathways.

Secreted interleukin-17A (sIL-17A) is a proinflammatory cytokine produced by T cells that induces cytokine production and mediates monocyte and neutrophil recruitment to sites of inflammation. IL-17A in the BT system is regulated by the calcineurin, EGFR, glucocorticoid receptor, HDAC, HMG-CoA reductase, histamine H1R, IKK2, JAK, MEK, microtubule, PI3K, PKC, Src, vitamin D receptor, mTOR, and p38 MAP pathways.

Secreted interleukin-17F (sIL-17F) is a proinflammatory cytokine produced by T cells that induces cytokine, chemokine, and adhesion molecule production and mediates neutrophil recruitment to sites of inflammation. IL-17F in the BT system is regulated by the calcineurin, EGFR, glucocorticoid receptor, HDAC, HMG-CoA reductase, histamine H1R, JAK, MEK, microtubule, PKC, RAR/RXR, Src, TNFα, vitamin D receptor, mTOR, and p38 MAP pathways.

Interleukin-6 (IL-6) is a secreted pro-inflammatory cytokine and acute phase reactant. Secreted IL-6 (sIL-6) is regulated in the BT system by the calcineurin, EGFR, glucocorticoid receptor, HDAC, histamine H1R, IKK2, JAK, MEK, PKC, Src, TNFα, vitamin D receptor, mTOR, and p38 MAPK pathways.

Tumor necrosis factor alpha (TNFα) is a secreted pro-inflammatory cytokine involved in Th1 vascular inflammation. Secreted TNFα (sTNFα) is regulated in the BT system by the calcineurin, EGFR, glucocorticoid receptor, HDAC, HMG-CoA Reductase, histamine H1R, IKK2, JAK, MEK, PI3K, PKC, RAR/RXR, Src, TNFα, vitamin D receptor, and mTOR pathways.

sIgG, sIL-17A, sIL-17F, sIL-6, and TNFα are categorized as immunomodulatory-related activities in the BT system modeling T cell dependent B cell activation.

Plasminogen activator inhibitor-1 (PAI-I) is a serine proteinase inhibitor and inhibitor of tissue plasminogen activator and urokinase and is involved in tissue remodeling and fibrinolysis. PAI-I is categorized as a tissue remodeling-related activity in the HDF3CGF system modeling Th1 inflammation involved in wound healing and matrix remodeling of the skin. PAI-I in the HDF3CGF system is regulated by the EGFR, histamine H1R, MEK, PDE4, PI3K, RAR/RXR, Src, and mTOR pathways.

As noted herein, the BioMAP data for 1-(((2S,3S,4S)-3-ethyl-4-fluoro-5-oxopyrrolidin-2-yl)methoxy)-7-methoxyisoquinoline-6-carboxamide showed reduced levels of pro-inflammatory molecules and immunomodulatory molecules in the LPS system (MCP-1, VCAM-1, CD40, E-selectin, CD69, IL-8, IL-1α, M-CSF, sPGE₂, and sTNFα) which models monocyte activation in response to TLR stimulation including robust inhibition of TNFα, a hallmark activation response for monocytes. This agent also had modest inhibitory effects in the BT system (sIL-17F and sTNFα) and in the CASM3C system (IL-6).

In contrast, tofacitinib displayed strong anti-inflammatory and immunomodulatory activities in the BT system (sIgG, sIL-17F, sIL-2, IL-6, and sTNFα) modeling T cell dependent B cell activation and also showed B cell anti-proliferative activity. In the 4H system at higher doses, tofacitinib inhibited: MCP-1 associated with recruitment of monocytes and T cells to sites of inflammation; Eto3 associated with recruitment of eosinphils and basophils to sites of inflammation; VCAM-1 associated with adhesion of monocytes and T cells to endothelial cells; and P-selectin related to mediation of platelet-endothelial and leukocyte-endothelial interactions. In the SAg system at higher doses, tofacitinib inhibited CD38, a cell surface enzyme involved in T cell activation/stimulation/chemotaxis, and CD69, a cell surface antigen involved in lymphocyte proliferation and activation. Also, at higher doses tofacitinib inhibited the chemokines MIG (CASM3C system), ITAC (HDF3CGF system), and MCP-1 (KF3CT system) involved in T cell recruitment. In the LPS system, tofacitinib stimulated production of IL-1α, sPGE₂ and sTNFα related to monocyte-driven Th1 vascular inflammation.

The BioMAP data generated for 1-(((2S,3S,4S)-3-ethyl-4-fluoro-5-oxopyrrolidin-2-yl)methoxy)-7-methoxyisoquinoline-6-carboxamide and tofacitinib demonstrated differences in their biological activities. 1-(((2S,3S,4S)-3-Ethyl-4-fluoro-5-oxopyrrolidin-2-yl)methoxy)-7-methoxyisoquinoline-6-carboxamide strongly inhibited activation responses in the LPS system modeling monocyte activation biology. In contrast, tofacitinib strongly inhibited responses in the BT system as well as having additional activities in systems stimulated by T cell activation (SAg) or cytokines (4H, CASM3C and HDF3CGF). These data showed that the two agents inhibited non-overlapping immune activation states related to monocyte versus T cell driven inflammation biology. This data supports the development of both agents together as a combination therapy for treating immune, autoimmune, and inflammatory disorders such as IBD, ulcerative colitis, Crohn's disease, vitiligo, and in particular rheumatoid arthritis in humans.

The activities of 1-(((2S,3S,4S)-3-Ethyl-4-fluoro-5-oxopyrrolidin-2-yl)methoxy)-7-methoxyisoquinoline-6-carboxamide and tofacitinib were evaluated separately and in combination in the 3C, SAg, and HDF3CGF BioMAP systems. The 3C system models vascular inflammation of the Th1 type, an environment that promotes monocyte and T cell adhesion and recruitment and is anti-angiogenic. This system is relevant for chronic inflammatory diseases, vascular inflammation and restenosis. The SAg system models chronic inflammation of the Th1 type and T cell effector responses to TCR signaling with co-stimulation. This system is relevant to inflammatory conditions where T cells play a role including transplantation, rheumatoid arthritis, psoriasis, Crohn's disease and multiple sclerosis. The HDF3CGF system models wound healing and matrix/tissue remodeling in the context of Th1-type inflammation. This system is relevant for various diseases including fibrosis, rheumatoid arthritis, psoriasis and stromal biology in tumors.

1-(((2S,3S,4S)-3-Ethyl-4-fluoro-5-oxopyrrolidin-2-yl)methoxy)-7-methoxyisoquinoline-6-carboxamide, alone, at nanomolar concentrations of 19, 56, 170, and 500 showed no activity in the 3C and SAg systems. In the HDF3CGF system, modest inhibition VCAM-1, Col-III, and TIMP2 were achieved at the highest dose.

Tofacitinib, alone, at nanomolar concentrations of 1000, 330, 110, and 37 showed no activity in the 3C system. At the highest concentration, tofacitinib modestly inhibited CD69 in the SAg system and VCAM-1 and ITAC in the HDF3CGF system.

The combination of 1-(((2S,3S,4S)-3-Ethyl-4-fluoro-5-oxopyrrolidin-2-yl)methoxy)-7-methoxyisoquinoline-6-carboxamide and tofacitinib showed an unexpected reduction in IL-8 levels in the SAg system modeling T cell driven vascular inflammation biology indicating cooperative interactions between these agents leading to a novel impact on disease biology. In the HDF3CGF system at the higher concentrations, the combination showed enhanced inhibition of VCAM-1 and a synergistic decrease in MIG levels.

These results demonstrated that 1-(((2S,3S,4S)-3-ethyl-4-fluoro-5-oxopyrrolidin-2-yl)methoxy)-7-methoxyisoquinoline-6-carboxamide and tofacitinib, separately, had different activities in the 3C, SAg, and HDF3CGF systems. In combination, these agents interacted together to unexpectedly reduce IL-8 levels in the SAg system, synergistically reduce MIG levels in the HDF3CGF system, and enhance reduction of VCAM-1 in the HDF3CGF system.

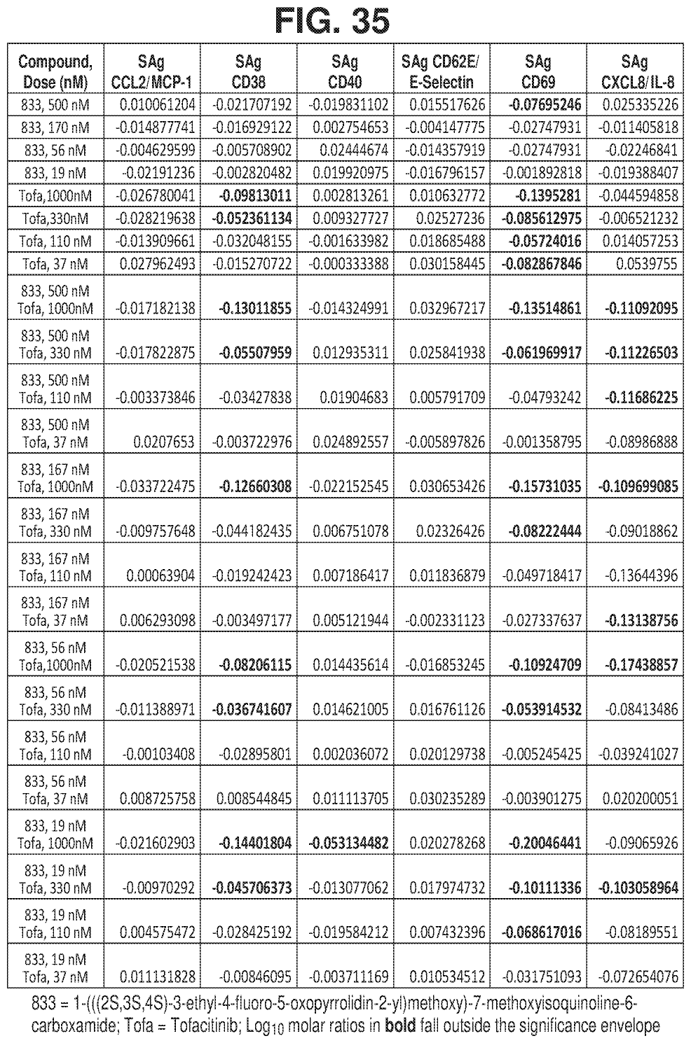

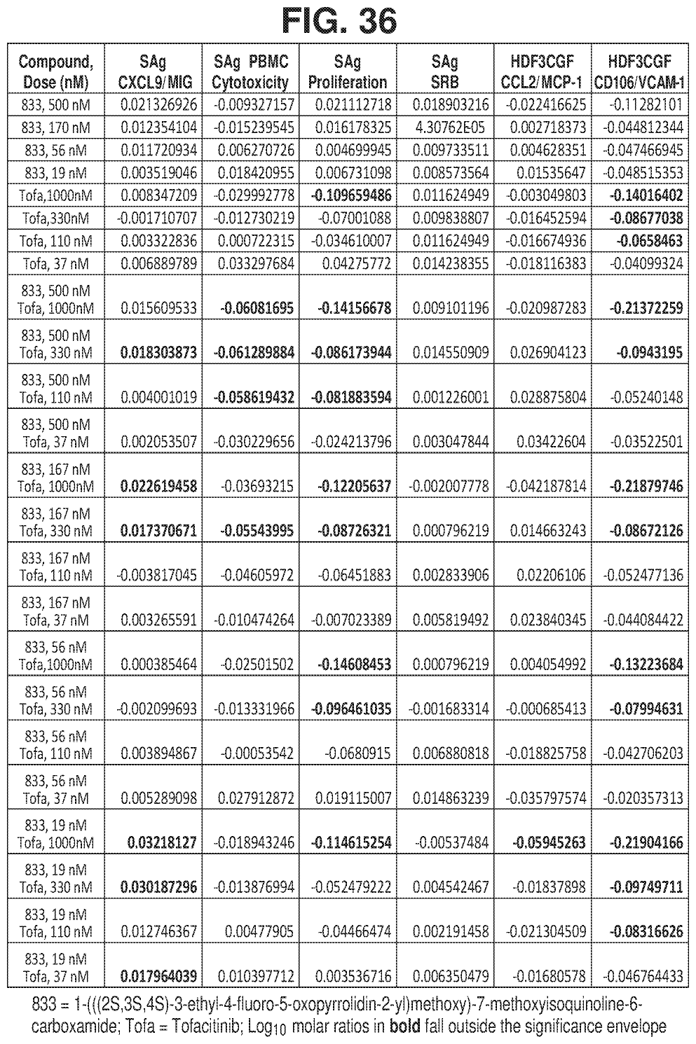

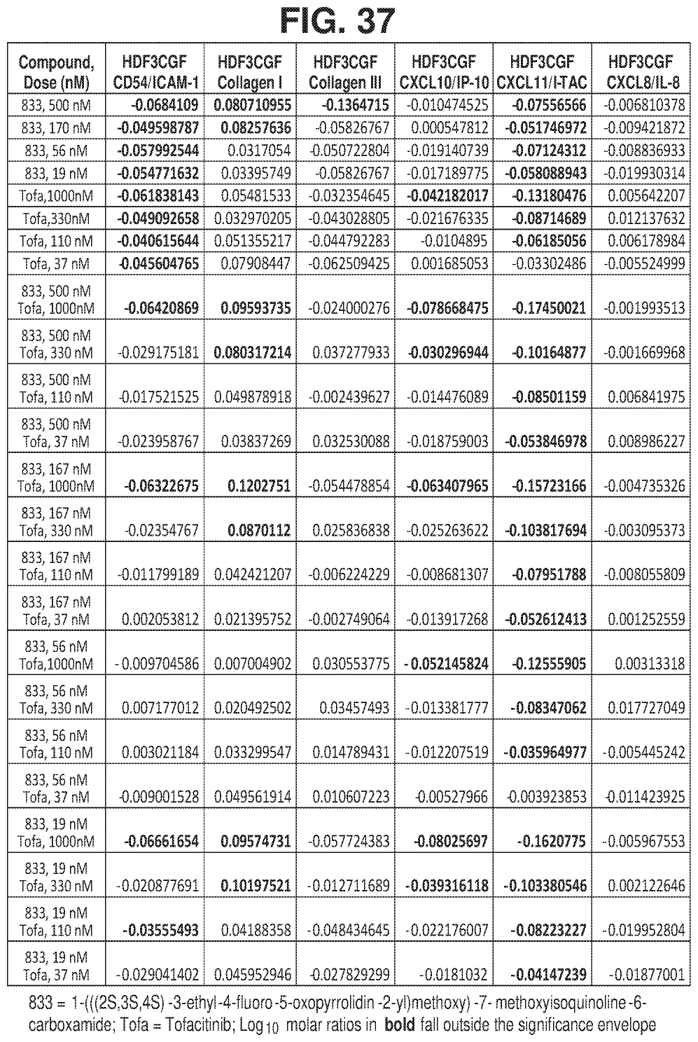

Regarding the IL-8 levels in the SAg system, both agents alone showed no inhibitory effects at all concentrations as the Log₁₀ molar ratios were all within the significance envelope. Unexpectedly, the combination of these agents at several different combination concentrations produced Log₁₀ molar ratios that indicated a reduction in IL-8 levels (FIG. 35). In the SAg system, IL-8 mediates neutrophil recruitment into acute inflammatory sites and also has a role with T cell-driven Th1 vascular inflammation.

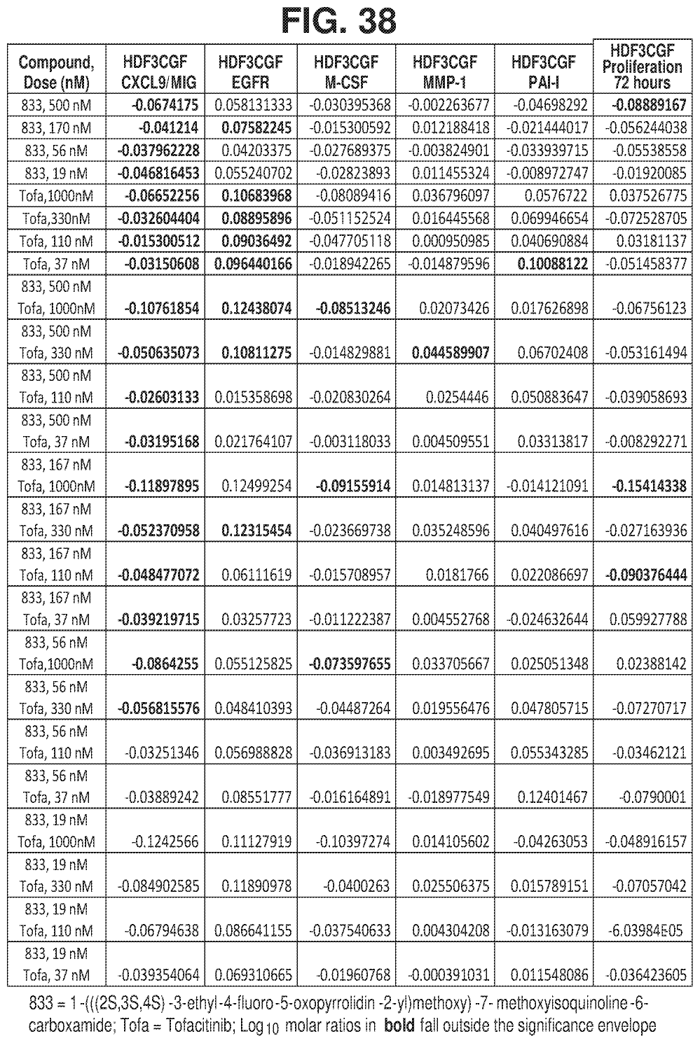

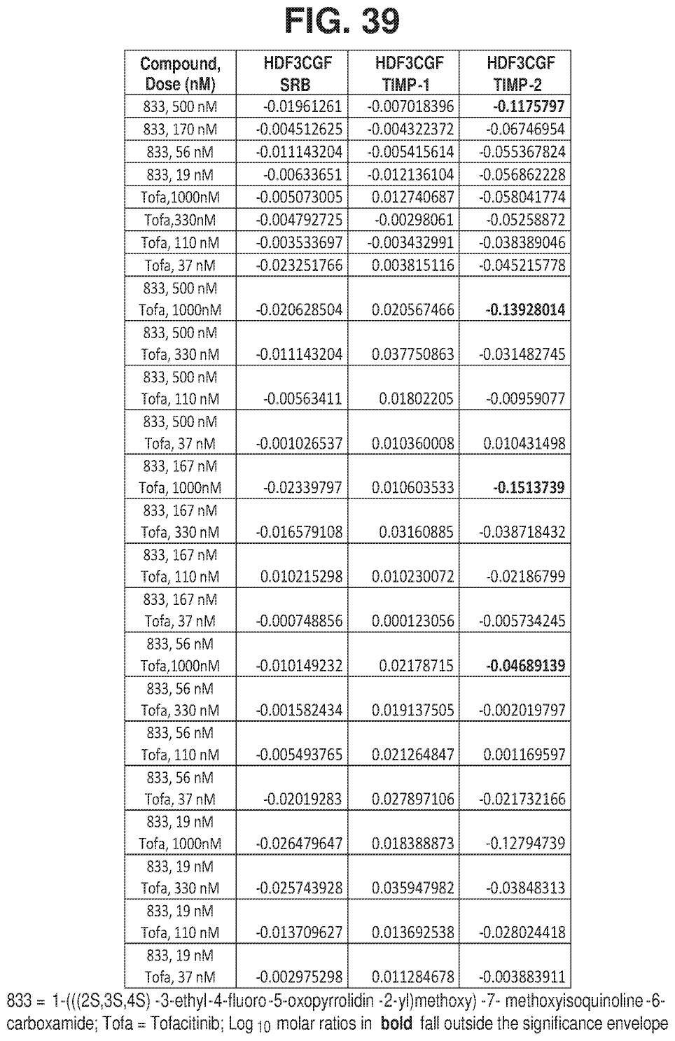

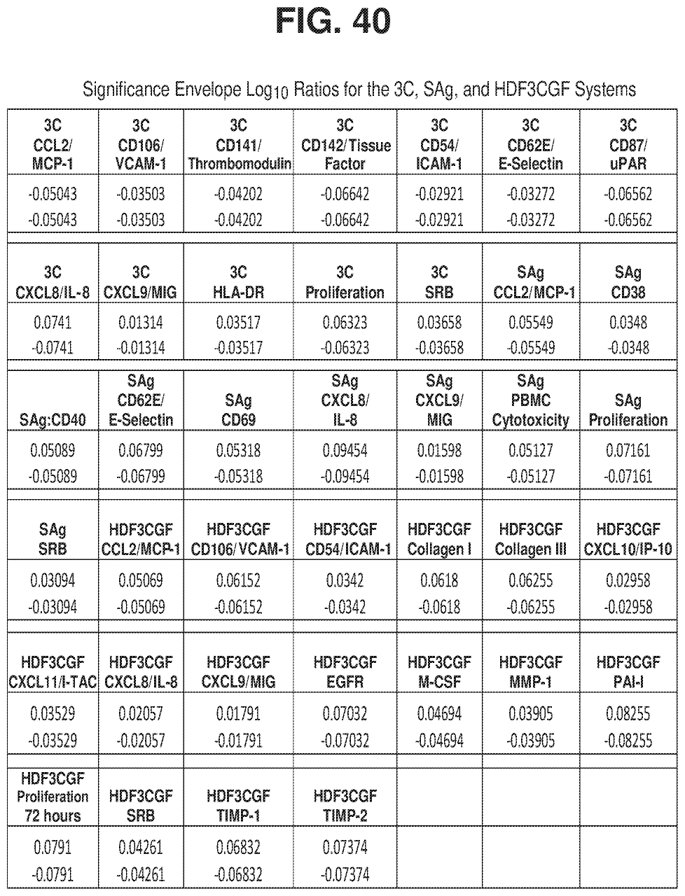

In the HDF3CGF system, the combination of 1-(((2S,3S,4S)-3-ethyl-4-fluoro-5-oxopyrrolidin-2-yl)methoxy)-7-methoxyisoquinoline-6-carboxamide at 170 nM and tofacitinib at 1000 nM showed a synergistic effect for reducing MIG levels. The Log₁₀ molar ratios for 1-(((2S,3S,4S)-3-ethyl-4-fluoro-5-oxopyrrolidin-2-yl)methoxy)-7-methoxyisoquinoline-6-carboxamide at 170 nM was −0.041214. The Log₁₀ molar ratio for tofacitinib at 1000 nM was −0.066522 resulting in an additive effect of −0.107736. The combination of 1-(((2S,3S,4S)-3-ethyl-4-fluoro-5-oxopyrrolidin-2-yl)methoxy)-7-methoxyisoquinoline-6-carboxamide at 167 nM and tofacitinib at 1000 nM unexpectedly resulted in a Log 10 molar ratio of −0.11897895, an unexpected synergistic result (FIG. 38). In the HDF3CGF system, monokine induced by gamma interferon (MIG) mediates T cell recruitment and has inflammatory activities related to wound healing and matrix remodeling of the skin.

As noted herein, the BioMAP data for 1-(((2S,3S,4S)-3-ethyl-4-fluoro-5-oxopyrrolidin-2-yl)methoxy)-7-methoxyisoquinoline-6-carboxamide showed reduced levels of pro-inflammatory molecules and immunomodulatory molecules in the LPS system (MCP-1, VCAM-1, CD40, E-selectin, CD69, IL-8, IL-1α, M-CSF, sPGE₂, and sTNFα) which models monocyte activation in response to TLR stimulation including robust inhibition of TNFα, a hallmark activation response for monocytes. This agent also had modest inhibitory effects in the BT system (sIL-17F and sTNFα) and in the CASM3C system (IL-6).

In contrast, 1-((2S,5R)-5-((7H-pyrrolo[2,3-d]pyrimidin-4-yl)amino)-2-methylpiperidin-1-yl)prop-2-en-1-one displayed strong anti-inflammatory and immunomodulatory activities in the BT system (sIgG, sIL-17A, sIL-17F, sIL-6, and sTNFα) modeling T cell dependent B cell activation and also showed B cell anti-proliferative activity. In the LPS system at higher doses, sTNFα levels were reduced. Also at higher doses in SAg system, sIL-s8 levels were reduced.

These results demonstrated that 1-(((2S,3S,4S)-3-ethyl-4-fluoro-5-oxopyrrolidin-2-yl)methoxy)-7-methoxyisoquinoline-6-carboxamide and 1-((2S,5R)-5-((7H-pyrrolo[2,3-d]pyrimidin-4-yl)amino)-2-methylpiperidin-1-yl)prop-2-en-1-one, separately, had different inhibitory activities in the LPS and BT systems. The different BioMAP profiles exhibited for these two agents support their use as a combination therapy for treating inflammatory, immune, and autoimmune diseases such as inflammatory bowel disease, ulcerative colitis, Crohn's disease, non-alcoholic steatohepatitis (NASH), liver fibrosis, non-alcoholic fatty liver disease (NAFLD), iodopathic pulmonary fibrosis (IPF), rheumatoid arthritis (RA), atopic dermatitis, psoriasis, psoriatic arthritis, stasis dermatitis, lupus, ankylosing spondylitis, alopecia, vitiligo, and hidradenitis suppurativa (HS), in particular, rheumatoid arthritis.

1. An oral dosage ECS single layer modified release tablet comprising 1-(((2S,3S,4S)-3-ethyl-4-fluoro-5-oxopyrrolidin-2-yl)methoxy)-7-methoxyisoquinoline-6-carboxamide (PF-06650833) or an equivalent amount of the PF-06650833 in the form of a pharmaceutically acceptable salt thereof, one or more osmogens, a suspending agent, a glidant, a tableting aid, and one or more lubricants as an active core, and a coating applied to the active core wherein the coating comprises an osmotic membrane and a plasticizer.

2. The oral dosage ECS single layer modified release tablet according to claim 1 wherein the osmogens are dextrates and sodium chloride, wherein the suspending agent is hydroxyethylcellulose, wherein glidant is colloidal silicon dioxide, wherein the tableting aid is copovidone, wherein the lubricants are magnesium stearate and sodium stearyl fumarate, wherein the osmotic membrane is cellulose acetate, and wherein the plasticizer is polyethylene glycol.

3. The oral dosage ECS single layer modified release tablet according to claim 2 wherein the dextrates amount is 275-385 mgs, wherein the sodium chloride amount is 150-250 mgs, wherein the hydroxyethylcellulose amount is 45-100 mgs, wherein the colloidal silicon dioxide amount is 1-5 mgs, wherein the copovidone amount is 60-120 mgs, wherein the magnesium stearate amount is 1-10 mgs, wherein the sodium stearyl fumarate amount is 1-10 mgs, wherein the cellulose acetate amount is 10-45 mgs, and wherein the polyethylene glycol amount is 1-20 mgs.

4. The oral dosage ECS single layer modified release tablet according to claim 2 wherein the dextrates amount is 320-340 mgs, wherein the sodium chloride amount is 195-215 mgs, wherein the hydroxyethylcellulose amount is 70-74 mgs, wherein the colloidal silicon dioxide amount is 2-2.5 mgs, wherein the copovidone amount is 79-83 mgs, wherein the magnesium stearate amount is 4-5 mgs, wherein the sodium stearyl fumarate amount is 4-5 mgs, wherein the cellulose acetate amount is 26-30 mgs, wherein the polyethylene glycol amount is 7-9 mgs, and wherein the PF-06650833 is unmilled.

5. The oral dosage ECS single layer modified release tablet according to claim 2 wherein the dextrates amount is 330 mgs, wherein the sodium chloride amount is 205 mgs, wherein the hydroxyethylcellulose amount is 72 mgs, wherein the colloidal silicon dioxide amount is 2.25 mgs, wherein the copovidone amount is 81 mgs, wherein the magnesium stearate amount is 4.5 mgs, wherein the sodium stearyl fumarate amount is 4.5 mgs, wherein the cellulose acetate amount is 28 mgs, wherein the polyethylene glycol amount is 8 mgs, and wherein the PF-06650833 is unmilled.

6. An oral dosage ECS single layer modified release tablet comprising 20-24% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 34-39% dextrates, 20-24% sodium chloride, 7-9% hydroxyethylcellulose, 0.20-0.30% mgs of colloidal silicon dioxide, 7-11% copovidone, 0.40-0.60% magnesium stearate, and 0.40-0.60% of sodium stearyl fumarate as an active core, and a coating applied to the active core wherein the coating comprises 75-81% cellulose acetate and 20-24% polyethylene glycol, wherein PF-06650833 is unmilled.

7. The oral dosage ECS single layer modified release tablet according to claim 6 comprising 22.22% 1-(((2S,3S,4S)-3-ethyl-4-fluoro-5-oxopyrrolidin-2-yl)methoxy)-7-methoxyisoquinoline-6-carboxamide or an equivalent amount of PF-06650833 in the form of a pharmaceutically acceptable salt thereof, 36.75% dextrates, 22.78% sodium chloride, 8.00% hydroxyethylcellulose, 0.25% mgs of colloidal silicon dioxide, 9.00% copovidone, 0.50% magnesium stearate, and 0.50% of sodium stearyl fumarate as an active core, and a coating applied to the active core wherein the coating comprises 78.00% cellulose acetate and 22.00% polyethylene glycol, wherein PF-06650833 is unmilled.

8. A method of treating or preventing hidradenitis suppurativa in a patient comprising administering orally to the patient in need of such treatment a therapeutically effective amount of 1-(((2S,3S,4S)-3-ethyl-4-fluoro-5-oxopyrrolidin-2-yl)methoxy)-7-methoxyisoquinoline-6-carboxamide or a pharmaceutically acceptable salt thereof.

9. The method according to claim 8 wherein the therapeutically effective amount is one 200 mg MR-FORM3 tablet taken orally once daily.

10. The method according to claim 8 wherein the therapeutically effective amount is two 200 mg MR-FORM3 tablets taken orally simultaneously or in sequence once daily.

11. A pharmaceutical combination comprising two 200 mg MR-FORM3 tablets and one 11 mg extended release tablet of tofacitinib.

12. A method of treating or preventing rheumatoid arthritis in a patient comprising administering orally to the patient in need of such treatment a pharmaceutical combination comprising two 200 mg MR-FORM3 tablets and one 11 mg extended release tablet of tofacitinib, taken simultaneously or in sequence once daily, wherein inflammatory activities from the innate and adaptive immune systems are reduced.

13. The method according to claim 12 wherein monocyte and B cell levels are reduced at the inflammation site.

14. The method according to claim 12 wherein IL-8 levels are reduced at the inflammation site.

15. The method according to claim 12 wherein neutrophil levels are reduced at the inflammation site.

16. The method according to claim 12 wherein monocyte, B cell, neutrophil, and IL-8 levels are reduced at the inflammation site.
